SEQUENCE LISTING (194 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 410 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 112 aa, Synthetic construct
EVQLQQSGADLVRPGALVKLSCKASGFNIKDYYLHWVRQRPEQGLEWIGWIDPENGDTVYDPKFQGKATITADTSSNTAYLQLGSLTSEDTAVYFCSTLDFWGQGTTLTVSS

SEQ ID NO: 8 - protein, 10 aa, Synthetic construct
GFNIKDYYLH

SEQ ID NO: 9 - protein, 17 aa, Synthetic construct
WIDPENGDTVYDPKFQG

SEQ ID NO: 10 - skipped

SEQ ID NO: 11 - protein, 112 aa, Synthetic construct
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKLEIK

SEQ ID NO: 12 - protein, 16 aa, Synthetic construct
KSSQSLLDSDGKTYLN

SEQ ID NO: 13 - protein, 7 aa, Synthetic construct
LVSKLDS

SEQ ID NO: 14 - protein, 9 aa, Synthetic construct
WQGTHFPYT

SEQ ID NO: 15 - protein, 112 aa, Synthetic construct
EVQLVQSGAEVVRPGALVKVSCKASGFNIKDYYLHWVRQAPEQGLEWIGWIDPENGDTVYDPKFQGKATITADTSTNTAYLQLSSLTSEDTAVYFCSTLDFWGQGTLVTVSS

SEQ ID NO: 16 - protein, 112 aa, Synthetic construct
EVQLVQSGAEVKKPGASVKVSCKVSGFNIKDYYLHWVRQAPEQGLEWMGWIDPENGDTVYDPKFQGRVTITADTSTNTAYMELSSLTSEDTAVYYCSTLDFWGQGTLVTVSS

SEQ ID NO: 17 - protein, 112 aa, Synthetic construct
EVQLVQSGAEVVRPGALVKISCKASGFNIKDYYLHWVRQRPEQGLEWIGWIDPENGDTVYDPKFQGKATITADTSTNTAYLQLGSLTSEDTAVYFCSTLDFWGQGTLVTVSS

SEQ ID NO: 18 - protein, 112 aa, Synthetic construct
EVQLVQSGAEVVKPGATVKISCKASGFNIKDYYLHWVRQRPGQGLEWIGWIDPENGDTVYDPKFQGRATITADTSTDTAYLQLGSLTSEDTAVYFCSTLDFWGQGTLVTVSS

SEQ ID NO: 19 - protein, 112 aa, Synthetic construct
EVQLVQSGAEVVKPGATVKISCKASGFTIKDYYLHWVRQRPGQGLEWIGWIDPEDGDTVYAPKFQGRATITADTSTDTAYLQLGSLTSEDTAVYFCSTLDFWGQGTLVTVSS

SEQ ID NO: 20 - protein, 112 aa, Synthetic construct
DVVMTQSPLSLSVTLGQPASISCKSSQSLLDSDGKTYLNWLLQRPGQSPKRLIYLVSKLDSGVPDRFSGSGSGTDFTLKISRVEAEDVGVYYCWQGTHFPYTFGGGTKLEIK

SEQ ID NO: 21 - protein, 112 aa, Synthetic construct
DVVMTQSPLSLPVTLGQPASISCKSSQSLLDSDGKTYLNWLLQRPGQSPRRLIYLVSKLDSGVPDRFSGSGSGTDFTLKISRVEAEDVGVYYCWQGTHFPYTFGGGTKLEIK

SEQ ID NO: 22 - protein, 112 aa, Synthetic construct
DVVMTQSPLSLPVTLGQPASISCKSSQSLLDSDGKTYLNWLLQRPGQSPRRLIYLVSKLDSGVPSRFSGSGSGTDFTLKISRVEAEDVGVYYCWQGTHFPYTFGGGTKLEIK

SEQ ID NO: 23 - protein, 112 aa, Synthetic construct
DIVMTQTPLSLSVTIGQPASISCKSSQSLLDSDGKTYLNWLLQKPGQSPKRLIYLVSKLDSGVPDRFSGSGSGTDFTLKISRVEAEDVGVYYCWQGTHFPYTFGGGTKLEIK

SEQ ID NO: 24 - protein, 119 aa, Homo sapiens
QVQLQQSGAEVKKPGSSVKVSCKASGGTFGSYAISWVRQAPGQGLEWMGRIIPILGIATYAQKFQGRVTITADKSTSTAYMDLSSLRSEDTAVYYCARGKGEFEGMDVWGQGTTVTVSS

SEQ ID NO: 25 - protein, 98 aa, Homo sapiens
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCAT

SEQ ID NO: 26 - protein, 13 aa, Homo sapiens
QHWGQGTLVTVSS

SEQ ID NO: 27 - protein, 100 aa, Homo sapiens
DVVMTQSPLSLPVTLGQPASISCRSSQSLVHSDGNTYLNWFQQRPGQSPRRLIYKVSNRDSGVPDRFSGSGSGTDFTLKISRVEAEDVGVYYCMQGTHWP

SEQ ID NO: 28 - protein, 12 aa, Homo sapiens
YTFGQGTKLEIK

SEQ ID NO: 29 - protein, 112 aa, Homo sapiens
DVVMTQSPLSLTVTLGQPASISCRSSQSLVYSDGNTYLNWFQQRPGQSPRRLIYRVSHWDSGVPDRFSGSGSGTDFTLKISRVEAEDVGVYYCMQGTYWPLTFGQGTKLEIK

SEQ ID NO: 30 - DNA, 336 bp, Synthetic construct
  source 1..336: mol_type = other DNA
gaggttcagctgcagcagtctggggctgaccttgtgaggccaggggccttagtcaagttgtcctgcaaagcttctggcttcaacattaaagactactatttgcactgggtgaggcagaggcctgaacagggcctggagtggattggatggattgatcctgagaatggtgatactgtatatgacccgaagttccagggcaaggccactataacagcagacacatcctccaatacagcctacctgcagctcggcagcctgacatctgaggacactgccgtctatttctgttctacccttgacttctggggccaaggcaccactctcacagtctcctca

SEQ ID NO: 31 - DNA, 339 bp, Synthetic construct
  source 1..339: mol_type = other DNA
gatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtacacgttcggaggggggaccaagctggaaataaaacgt

SEQ ID NO: 32 - protein, 5 aa, Synthetic construct
DYYLH

SEQ ID NO: 33 - protein, 7 aa, Synthetic construct
GFNIKDY

SEQ ID NO: 34 - protein, 6 aa, Synthetic construct
DPENGD

SEQ ID NO: 35 - protein, 10 aa, Synthetic construct
WIDPENGDTV

SEQ ID NO: 36 - protein, 7 aa, Synthetic construct
KTYLNWL

SEQ ID NO: 37 - protein, 10 aa, Synthetic construct
RLIYLVSKLD

SEQ ID NO: 38 - protein, 8 aa, Synthetic construct
WQGTHFPY

SEQ ID NO: 39 - protein, 6 aa, Synthetic construct
KDYYLH

SEQ ID NO: 40 - protein, 13 aa, Synthetic construct
WIGWIDPENGDTV

SEQ ID NO: 41 - protein, 4 aa, Synthetic construct
STLD

SEQ ID NO: 42 - protein, 10 aa, Synthetic construct
GFTIKDYYLH

SEQ ID NO: 43 - protein, 17 aa, Synthetic construct
WIDPEDGDTVYAPKFQG

SEQ ID NO: 44 - protein, 112 aa, Synthetic construct
  VARIANT 13: note = Arg or Lys
  VARIANT 67: note = Lys or Arg
EVQLVQSGAEVVXPGALVKISCKASGFNIKDYYLHWVRQRPEQGLEWIGWIDPENGDTVYDPKFQGXATITADTSTNTAYLQLGSLTSEDTAVYFCSTLDFWGQGTLVTVSS

SEQ ID NO: 45 - protein, 113 aa, Synthetic construct
DVVMTQSPLSLSVTLGQPASISCKSSQSLLDSDGKTYLNWLLQRPGQSPKRLIYLVSKLDSGVPDRFSGSGSGTDFTLKISRVEAEDVGVYYCWQGTHFPYTFGGGTKLEIKR

SEQ ID NO: 46 - protein, 112 aa, Synthetic construct
EVQLVQSGAEVVKPGATVKISCKASGFTIKDYYLHWVRQRPGKGLEWIGWVDPEDGDTVYAPKFQGRATITADTSTDTAYLELGSLTSEDTAVYFCSTLDFWGQGTLVTVSS

SEQ ID NO: 47 - protein, 112 aa, Synthetic construct
EVQLVQSGAEVVKPGATVKISCKASGFTIKDYYLHWVRQRPGKGLEWIGWIDPEDGDTVYAPKFQGRATITADTSTDTAYLELGSLTSEDTAVYFCSTLDFWGQGTLVTVSS

SEQ ID NO: 48 - protein, 112 aa, Synthetic construct
EVQLVQSGAEVKRPGALVKISCKASGFNFKDYYLHWVRQRPEQGLEWMGWIDPENGDTVYDEKFQGRVTITADTSTNTAYLQLGSLTSEDTAVYFCSTLDFWGQGTLVTVSS

SEQ ID NO: 49 - protein, 112 aa, Synthetic construct
EVQLVQSGAEVKRPGALVKISCKASGYTFTDYYLHWVRQRPEQGLEWMGWVDPEDGDTVYAEKFQGRVTITADTSTNTAYLQLGSLTSEDTAVYFCSTLDFWGQGTLVTVSS

SEQ ID NO: 50 - protein, 112 aa, Synthetic construct
EVQLVQSGADVVKPGALVKISCKASGFTIKDYYLHWVRQRPEQGLEWIGWIDPENGDTVYAEKFQGRVTITADTSTNTAYLELGSLTSEDTAVYFCSTLDFWGQGTTLTVSS

SEQ ID NO: 51 - protein, 112 aa, Synthetic construct
EVQLVQSGADVVKPGALVKISCKASGFTIKDYYLHWVRQRPGQGLEWIGWVDPEDGDTVYAEKFQGRVTITADTSTDTAYMELGSLTSEDTAVYFCSTLDYWGQGTTLTVSS

SEQ ID NO: 52 - protein, 112 aa, Synthetic construct
EVQLVQSGAEVKKPGATVKISCKVSGFNIKDYYLHWVRQAPGKGLEWMGWIDPENGDTVYDPKFQGRVTITADTSTDTAYMELSSLRSEDTAVYYCSTLDFWGQGTLVTVSS

SEQ ID NO: 53 - protein, 112 aa, Synthetic construct
EVQLVQSGAEVKKPGALVKISCKVSGYNFKDYYLHWVRQAPGKGLEWMGWIDPENGDTVYDEKFQGRVTITADTSTNTAYMELGSLRSEDTAVYYCSTLDFWGQGTLVTVSS

SEQ ID NO: 54 - protein, 112 aa, Synthetic construct
EVQLVQSGAEVKKPGALVKISCKVSGYTFTDYYLHWVRQAPGKGLEWMGWVDPEDGDTVYAEKFQGRVTITADTSTNTAYMELGSLRSEDTAVYYCSTLDFWGQGTLVTVSS

SEQ ID NO: 55 - protein, 112 aa, Synthetic construct
EVQLVQSGAEVVKPGATVKISCKVSGFNIKDYYLHWVRQRPGKGLEWIGWIDPENGDTVYDPKFQGKATITADTSTNTAYLELGSLTSEDTAVYYCSTLDFWGQGTLVTVSS

SEQ ID NO: 56 - protein, 112 aa, Synthetic construct
EVQLVQSGAEVVKPGALVKISCKVSGYNFKDYYLHWVRQRPGKGLEWMGWIDPENGDTVYDEKFQGRVTITADTSTDTAYLELGSLTSEDTAVYYCSTLDFWGQGTLVTVSS

SEQ ID NO: 57 - protein, 112 aa, Synthetic construct
EVQLVQSGAEVVKPGALVKISCKVSGYTFTDYYLHWVRQRPGKGLEWMGWVDPEDGDTVYAEKFQGRVTITADTSTDTAYLELGSLTSEDTAVYYCSTLDFWGQGTLVTVSS

SEQ ID NO: 58 - protein, 10 aa, Synthetic construct
GFNFKDYYLH

SEQ ID NO: 59 - protein, 10 aa, Synthetic construct
GYTFTDYYLH

SEQ ID NO: 60 - protein, 10 aa, Synthetic construct
GYNFKDYYLH

SEQ ID NO: 61 - protein, 17 aa, Synthetic construct
WVDPEDGDTVYAPKFQG

SEQ ID NO: 62 - protein, 17 aa, Synthetic construct
WIDPENGDTVYDEKFQG

SEQ ID NO: 63 - protein, 17 aa, Synthetic construct
WVDPEDGDTVYAEKFQG

SEQ ID NO: 64 - protein, 17 aa, Synthetic construct
WIDPENGDTVYAEKFQG

SEQ ID NO: 65 - skipped

SEQ ID NO: 66 - protein, 112 aa, Synthetic construct
EVQLQQSGAELVRSGASVKLSCTASGLNIKDYYIHWVKQRPEQGLEWIGWIDPENDDTEYAPKFQGRATLTTDTSSNTAYLQLSSLTSEDTAVYYCTPLDYWGQGTSVTVSS

SEQ ID NO: 67 - protein, 10 aa, Synthetic construct
GLNIKDYYIH

SEQ ID NO: 68 - protein, 17 aa, Synthetic construct
WIDPENDDTEYAPKFQG

SEQ ID NO: 69 - skipped

SEQ ID NO: 70 - protein, 114 aa, Mus musculus
KVKLQQSGAELVRSGASVKLSCTASGFNIKDYYIQWVKQRPEQGLEWIGWIDPENGNSEYAPRFQGKATMTADTLSNTAYLQLSSLTSEDTAVYYCNADLHDYWGQGTTLTVSS

SEQ ID NO: 71 - protein, 112 aa, Synthetic construct
EVQLVQSGAEVVKPGALVKISCKASGFNIKDYYLHWVRQRPGQGLEWIGWIDPENGDTVYDPKFQGRVTITADTSTNTAYLELGSLTSEDTAVYFCSTLDFWGQGTLVTVSS

SEQ ID NO: 72 - protein, 442 aa, Synthetic construct
EVQLQQSGADLVRPGALVKLSCKASGFNIKDYYLHWVRQRPEQGLEWIGWIDPENGDTVYDPKFQGKATITADTSSNTAYLQLGSLTSEDTAVYFCSTLDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 219 aa, Synthetic construct
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 115 aa, Homo sapiens
EVQLQQSGAELVRPGSSVKISCKASGYIFNNYWINWVKQRPGQGLEWIGQIYPGDGDTNYNGKFKGKATLTADKSSSTAYMQLSSLTSEDSAVYFCAREGYIVYWGQGTLVTVSA

SEQ ID NO: 75 - protein, 114 aa, Homo sapiens
QVQLQQSGAELVKPGASVKLSCTASGFNIKDTYMHWVKQRPEQGLEWIGRIDPANGNTKYDPKFQGKATITADTSSNTAYLQLSSLTSEDTAVYYCASYYGIYWGQGTTLTVSS

SEQ ID NO: 76 - protein, 112 aa, Synthetic construct
QVQLQQSGAELVKPGASVKLSCTASGFNIKDYYLHWVKQRPEQGLEWIGWIDPENGDTVYDPKFQGKATITADTSSNTAYLQLSSLTSEDTAVYFCSTLDFWGQGTTLTVSS

SEQ ID NO: 77 - protein, 112 aa, Synthetic construct
EVQLVQSGAEVVKPGASVKISCKASGFNIKDYYLHWVRQRPGKGLEWIGWIDPENGDTVYDPKFQGRATITADTSTDTAYLELSSLTSEDTAVYFCSTLDFWGQGTLVTVSS

SEQ ID NO: 78 - protein, 112 aa, Synthetic construct
EVQLVQSGAEVVKPGATVKISCKASGFNIKDYYLHWVRQRPGKGLEWIGWIDPENGDTIYDPKFQGRATITADTSTDTAYMELSSLRSEDTAVYYCSTLDFWGQGTLVTVSS

SEQ ID NO: 79 - protein, 112 aa, Synthetic construct
EVQLVQSGAEVVKPGATVKISCKASGFTIKDYYLHWVRQRPGKGLEWIGWIDPENGDTIYDPKFQGRVTITADTSTDTAYMELSSLRSEDTAVYYCSTLDFWGQGTLVTVSS

SEQ ID NO: 80 - protein, 112 aa, Synthetic construct
EVQLVQSGAEVVKPGATVKISCKASGFTIKDYYLHWVRQRPGKGLEWIGWIDPEDGETIYDPKFQGRVTITADTSTDTAYMELSSLRSEDTAVYYCSTLDFWGQGTLVTVSS

SEQ ID NO: 81 - protein, 112 aa, Homo sapiens
DVVLTQTPLTLSVTIGQPASISCKSSQSLLYSNGKTYLNWLLQRPGQSPKRLIYVVSKLDSGVPDRFTGSGSGTDFTLKISRVEAEDLGVYYCVQGTHFPFTFGSGTKLEIK

SEQ ID NO: 82 - protein, 112 aa, Homo sapiens
DVVMTQTPLTLSVTIGQPASISCKSSQSLLYSNGKTYLNWLLQRPGQSPKRLIYLVSKLDSGVPDRFSGSGSGTDFTLKISRVEAEDLGVHYCEQGTHFPLTFGAGTKLELK

SEQ ID NO: 83 - protein, 112 aa, Synthetic construct
DVVMTQTPLTLSVTIGQPASISCKSSQSLLDSDGKTYLNWLLQRPGQSPKRLIYLVSKLDSGVPDRFSGSGSGTDFTLKISRVEAEDLGVHYCWQGTHFPYTFGAGTKLELK

SEQ ID NO: 84 - protein, 112 aa, Synthetic construct
DVVMTQSPLSLSVTLGQPASISCKSSQSLLDSDGKTYLNWLLQRPGQSPKRLIYLVSKLDSGVPDRFSGSGSGTDFTLKISRVEAEDVGVYYCWQGTHFPYTFGAGTKLEIK

SEQ ID NO: 85 - protein, 112 aa, Synthetic construct
DVVMTQSPLSLSVTLGEPASISCRSSQSLLDSDGKTYLNWLQQRPGQSPRRLIYLVSKLDSGVPDRFSGSGSGTDFTLKISRVEAEDVGVYYCWQGTHFPYTFGQGTKLEIK

SEQ ID NO: 86 - protein, 10 aa, Synthetic construct
GFTIKDYYLH

SEQ ID NO: 87 - protein, 17 aa, Synthetic construct
WIDPENGDTIYDPKFQG

SEQ ID NO: 88 - protein, 17 aa, Synthetic construct
WIDPEDGETIYDPKFQG

SEQ ID NO: 89 - protein, 16 aa, Synthetic construct
RSSQSLLDSDGKTYLN

SEQ ID NO: 90 - protein, 112 aa, Synthetic construct
EVQLVQSGAEVVKPGATVKISCKASGFTIKDYYLHWVRQRPGKGLEWIGWIDPEDGETVYDPKFQGRVTITADTSTDTAYMELSSLRSEDTAVYFCSTLDFWGQGTLVTVSS

SEQ ID NO: 91 - protein, 112 aa, Synthetic construct
EVQLVQSGAEVVKPGATVKISCKASGFTIKDYYLHWVRQRPGKGLEWIGWIDPEDGETVYDPKFQGRVTITADTSTDTAYMELSSLRSEDTAVYYCSTLDFWGQGTLVTVSS

SEQ ID NO: 92 - protein, 17 aa, Synthetic construct
WIDPEDGETVYDPKFQG

SEQ ID NO: 93 - protein, 112 aa, Synthetic construct
DVVMTQSPLSLPVTLGQPASISCKSSQSLLDSDGKTYLNWLLQRPGQSPRRLIYLVSKDDSGVPDRFSGSGSGTDFTLKISRVEAEDVGVYYCWQGTHFPYTFGGGTKLEIK

SEQ ID NO: 94 - protein, 112 aa, Synthetic construct
DVVMTQSPLSLPVTLGQPASISCKSSQSLLDSDGKTYLNWLLQRPGQSPRRLIYLVSKGDSGVPDRFSGSGSGTDFTLKISRVEAEDVGVYYCWQGTHFPYTFGGGTKLEIK

SEQ ID NO: 95 - protein, 112 aa, Synthetic construct
DVVMTQSPLSLPVTLGQPASISCKSSQSLLDSDGKTYLNWLLQRPGQSPRRLIYLVSKNDSGVPDRFSGSGSGTDFTLKISRVEAEDVGVYYCWQGTHFPYTFGGGTKLEIK

SEQ ID NO: 96 - protein, 112 aa, Synthetic construct
DVVMTQSPLSLPVTLGQPASISCKSSQSLLDSDGKTYLNWLLQRPGQSPRRLIYLVSKEDSGVPDRFSGSGSGTDFTLKISRVEAEDVGVYYCWQGTHFPYTFGGGTKLEIK

SEQ ID NO: 97 - protein, 112 aa, Synthetic construct
DVVMTQSPLSLPVTLGQPASISCKSSQSLLDSDGKTYLNWLLQRPGQSPRRLIYEVSKLDSGVPDRFSGSGSGTDFTLKISRVEAEDVGVYYCWQGTHFPYTFGGGTKLEIK

SEQ ID NO: 98 - protein, 112 aa, Synthetic construct
DVVMTQSPLSLPVTLGQPASISCKSSQSLLDSDGKTYLNWLLQRPGQSPRRLIYLVSKQDSGVPDRFSGSGSGTDFTLKISRVEAEDVGVYYCWQGTHFPYTFGGGTKLEIK

SEQ ID NO: 99 - protein, 112 aa, Synthetic construct
DVVMTQSPLSLPVTLGQPASISCKSSQSLLDSDGKTYLNWLLQRPGQSPRRLIYDVSKLDSGVPDRFSGSGSGTDFTLKISRVEAEDVGVYYCWQGTHFPYTFGGGTKLEIK

SEQ ID NO: 100 - protein, 112 aa, Synthetic construct
DVVMTQSPLSLPVTLGQPASISCKSSQSLLDSDGKTYLNWLLQRPGQSPRRLIYLVSKKDSGVPDRFSGSGSGTDFTLKISRVEAEDVGVYYCWQGTHFPYTFGGGTKLEIK

SEQ ID NO: 101 - protein, 112 aa, Synthetic construct
DVVMTQSPLSLPVTLGQPASISCKSSQSLLDSDGKTYLNWLLQRPGQSPRRLIYLVSKRDSGVPDRFSGSGSGTDFTLKISRVEAEDVGVYYCWQGTHFPYTFGGGTKLEIK

SEQ ID NO: 102 - protein, 112 aa, Synthetic construct
DVVMTQSPLSLPVTLGQPASISCKSSQSLLDSDGKTYLNWLLQRPGQSPRRLIYLVSKTDSGVPDRFSGSGSGTDFTLKISRVEAEDVGVYYCWQGTHFPYTFGGGTKLEIK

SEQ ID NO: 103 - protein, 112 aa, Synthetic construct
DVVMTQSPLSLPVTLGQPASISCKSSQSLLDSDGKTYLNWLLQRPGQSPRRLIYGVSKLDSGVPDRFSGSGSGTDFTLKISRVEAEDVGVYYCWQGTHFPYTFGGGTKLEIK

SEQ ID NO: 104 - protein, 112 aa, Synthetic construct
DVVMTQSPLSLPVTLGQPASISCKSSQSLLDSDGKTYLNWLLQRPGQSPRRLGYLVSKLDSGVPDRFSGSGSGTDFTLKISRVEAEDVGVYYCWQGTHFPYTFGGGTKLEIK

SEQ ID NO: 105 - protein, 112 aa, Synthetic construct
DVVMTQSPLSLPVTLGQPASISCKSSQSLLDSDGKTYLNWLLQRPGQSPRRLDYLVSKLDSGVPDRFSGSGSGTDFTLKISRVEAEDVGVYYCWQGTHFPYTFGGGTKLEIK

SEQ ID NO: 106 - protein, 112 aa, Synthetic construct
DVVMTQSPLSLPVTLGQPASISCKSSQSLLDSDGKTYLNWLLQRPGQSPRRGIYLVSKLDSGVPDRFSGSGSGTDFTLKISRVEAEDVGVYYCWQGTHFPYTFGGGTKLEIK

SEQ ID NO: 107 - protein, 112 aa, Synthetic construct
DVVMTQSPLSLPVTLGQPASISCKSSQSLLDSDGKTYLNWLLQRPGQSPRRLIELVSKLDSGVPDRFSGSGSGTDFTLKISRVEAEDVGVYYCWQGTHFPYTFGGGTKLEIK

SEQ ID NO: 108 - protein, 112 aa, Synthetic construct
DVVMTQSPLSLPVTLGQPASISCKSSQSLLDSDGKTYLNWLLQRPGQSPRRLIYLVSKVDSGVPDRFSGSGSGTDFTLKISRVEAEDVGVYYCWQGTHFPYTFGGGTKLEIK

SEQ ID NO: 109 - protein, 112 aa, Synthetic construct
DVVMTQSPLSLPVTLGQPASISCKSSQSLLDSDGKTYLNWLLQRPGQSPRRLIYLVSKSDSGVPDRFSGSGSGTDFTLKISRVEAEDVGVYYCWQGTHFPYTFGGGTKLEIK

SEQ ID NO: 110 - protein, 112 aa, Synthetic construct
DVVMTQSPLSLPVTLGQPASISCKSSQSLLDSDGKTYLNWLLQRPGQSPRRLIYLVGKLDSGVPDRFSGSGSGTDFTLKISRVEAEDVGVYYCWQGTHFPYTFGGGTKLEIK

SEQ ID NO: 111 - protein, 112 aa, Synthetic construct
DVVMTQSPLSLPVTLGQPASISCKSSQSLLDSDGKTYLNWLLQRPGQSPRRNIYLVSKLDSGVPDRFSGSGSGTDFTLKISRVEAEDVGVYYCWQGTHFPYTFGGGTKLEIK

SEQ ID NO: 112 - protein, 112 aa, Synthetic construct
DVVMTQSPLSLPVTLGQPASISCKSSQSLLDSDGKTYLNWLLQRPGQSPRRDIYLVSKLDSGVPDRFSGSGSGTDFTLKISRVEAEDVGVYYCWQGTHFPYTFGGGTKLEIK

SEQ ID NO: 113 - protein, 112 aa, Synthetic construct
DVVMTQSPLSLPVTLGQPASISCKSSQSLLDSDGKTYLNWLLQRPGQSPRREIYLVSKLDSGVPDRFSGSGSGTDFTLKISRVEAEDVGVYYCWQGTHFPYTFGGGTKLEIK

SEQ ID NO: 114 - protein, 112 aa, Synthetic construct
DVVMTQSPLSLPVTLGQPASISCKSSQSLLDSDGKTYLNWLLQRPGQSPRRPIYLVSKLDSGVPDRFSGSGSGTDFTLKISRVEAEDVGVYYCWQGTHFPYTFGGGTKLEIK

SEQ ID NO: 115 - protein, 112 aa, Synthetic construct
DVVMTQSPLSLPVTLGQPASISCKSSQSLLDSDGKTYLNWLLQRPGQSPRRTIYLVSKLDSGVPDRFSGSGSGTDFTLKISRVEAEDVGVYYCWQGTHFPYTFGGGTKLEIK

SEQ ID NO: 116 - protein, 112 aa, Synthetic construct
DVVMTQSPLSLPVTLGQPASISCKSSQSLLDSDGKTYLNWLLQRPGQSPRRSIYLVSKLDSGVPDRFSGSGSGTDFTLKISRVEAEDVGVYYCWQGTHFPYTFGGGTKLEIK

SEQ ID NO: 117 - protein, 112 aa, Synthetic construct
DVVMTQSPLSLPVTLGQPASISCKSSQSLLDSDGKTYLNWLLQRPGQSPRRAIYLVSKLDSGVPDRFSGSGSGTDFTLKISRVEAEDVGVYYCWQGTHFPYTFGGGTKLEIK

SEQ ID NO: 118 - protein, 112 aa, Synthetic construct
DVVMTQSPLSLPVTLGQPASISCKSSQSLLDSDGKTYLNWLLQRPGQSPRRLIYVVSKLDSGVPDRFSGSGSGTDFTLKISRVEAEDVGVYYCWQGTHFPYTFGGGTKLEIK

SEQ ID NO: 119 - protein, 112 aa, Synthetic construct
DVVMTQSPLSLPVTLGQPASISCKSSQSLLDSDGKTYLNWLQQRPGQSPRRLIYGVSKGDSGVPDRFSGSGSGTDFTLKISRVEAEDVGVYYCWQGTHFPYTFGGGTKLEIK

SEQ ID NO: 120 - protein, 112 aa, Synthetic construct
DVVMTQSPLSLPVTLGQPASISCKSSQSLLDSDGKTYLNWLQQRPGQSPRRLIYEVSKGDSGVPDRFSGSGSGTDFTLKISRVEAEDVGVYYCWQGTHFPYTFGGGTKLEIK

SEQ ID NO: 121 - protein, 112 aa, Synthetic construct
DVVMTQSPLSLPVTLGQPASISCKSSQSLLDSDGKTYLNWLQQRPGQSPRRLIYLVGKGDSGVPDRFSGSGSGTDFTLKISRVEAEDVGVYYCWQGTHFPYTFGGGTKLEIK

SEQ ID NO: 122 - protein, 112 aa, Synthetic construct
DVVMTQSPLSLPVTLGQPASISCKSSQSLLDSDGKTYLNWLQQRPGQSPRRLIYLVGKRDSGVPDRFSGSGSGTDFTLKISRVEAEDVGVYYCWQGTHFPYTFGGGTKLEIK

SEQ ID NO: 123 - protein, 112 aa, Synthetic construct
DVVMTQSPLSLPVTLGQPASISCKSSQSLLDSDGKTYLNWLQQRPGQSPRRLIYLVGKTDSGVPDRFSGSGSGTDFTLKISRVEAEDVGVYYCWQGTHFPYTFGGGTKLEIK

SEQ ID NO: 124 - protein, 112 aa, Synthetic construct
DVVMTQSPLSLPVTLGQPASISCKSSQSLLDSDGKTYLNWLQQRPGQSPRRLIYLVGKDDSGVPDRFSGSGSGTDFTLKISRVEAEDVGVYYCWQGTHFPYTFGGGTKLEIK

SEQ ID NO: 125 - protein, 112 aa, Synthetic construct
DVVMTQSPLSLPVTLGQPASISCKSSQSLLDSDGKTYLNWLQQRPGQSPRRLIYLVSKGDSGVPDRFSGSGSGTDFTLKISRVEAEDVGVYYCWQGTHFPYTFGGGTKLEIK

SEQ ID NO: 126 - protein, 112 aa, Synthetic construct; identical to SEQ ID NO: 125

SEQ ID NO: 127 - protein, 112 aa, Synthetic construct
DVVMTQSPLSLPVTLGQPASISCKSSQSLLDSDGKTYLNWLQQRPGQSPRRLIYLVSKDDSGVPDRFSGSGSGTDFTLKISRVEAEDVGVYYCWQGTHFPYTFGGGTKLEIK

SEQ ID NO: 128 - protein, 112 aa, Synthetic construct
DVVMTQSPLSLPVTLGQPASISCKSSQSLLDSDGKTYLNWLQQRPGQSPRRLIYGVSKLDSGVPDRFSGSGSGTDFTLKISRVEAEDVGVYYCWQGTHFPYTFGGGTKLEIK

SEQ ID NO: 129 - protein, 112 aa, Synthetic construct
DVVMTQSPLSLPVTLGQPASISCKSSQSLLDSDGKTYLNWLQQRPGQSPRRLIYDVSKLDSGVPDRFSGSGSGTDFTLKISRVEAEDVGVYYCWQGTHFPYTFGGGTKLEIK

SEQ ID NO: 130 - protein, 112 aa, Synthetic construct; identical to SEQ ID NO: 129

SEQ ID NO: 131 - protein, 112 aa, Synthetic construct
DVVMTQSPLSLPVTLGQPASISCKSSQSLLDSDGKTYLNWLQQRPGQSPRRLIYLVGKLDSGVPDRFSGSGSGTDFTLKISRVEAEDVGVYYCWQGTHFPYTFGGGTKLEIK

SEQ ID NO: 132 - protein, 112 aa, Synthetic construct
DVVMTQSPLSLPVTLGQPASISCKSSQSLLDSDGKTYLNWLQQRPGQSPRRLIYDVSKGDSGVPDRFSGSGSGTDFTLKISRVEAEDVGVYYCWQGTHFPYTFGGGTKLEIK

SEQ ID NO: 133 - protein, 112 aa, Synthetic construct
DVVMTQSPLSLPVTLGQPASISCKSSQSLLDSDGKTYLNWLQQRPGQSPRRLIYDVSKRDSGVPDRFSGSGSGTDFTLKISRVEAEDVGVYYCWQGTHFPYTFGGGTKLEIK

SEQ ID NO: 134 - protein, 112 aa, Synthetic construct; identical to SEQ ID NO: 120

SEQ ID NO: 135 - protein, 112 aa, Synthetic construct
DVVMTQSPLSLPVTLGQPASISCKSSQSLLDSDGKTYLNWLQQRPGQSPRRLIYEVSKRDSGVPDRFSGSGSGTDFTLKISRVEAEDVGVYYCWQGTHFPYTFGGGTKLEIK

SEQ ID NO: 136 - protein, 112 aa, Synthetic construct
DVVMTQSPLSLPVTLGQPASISCKSSQSLLDSDGKTYLNWLQQRPGQSPRRLIYGVSKRDSGVPDRFSGSGSGTDFTLKISRVEAEDVGVYYCWQGTHFPYTFGQGTKLEIK

SEQ ID NO: 137 - protein, 112 aa, Synthetic construct
DVVMTQSPLSLPVTLGQPASISCKSSQSLLDSDGKTYLNWLQQRPGQSPRRLIYGVSKGDSGVPDRFSGSGSGTDFTLKISRVEAEDVGVYYCWQGTHFPYTFGQGTKLEIK

SEQ ID NO: 138 - protein, 112 aa, Synthetic construct
DVVMTQSPLSLPVTLGQPASISCKSSQSLLDSDGKTYLNWLQQRPGQSPRRLIYLVGKRDSGVPDRFSGSGSGTDFTLKISRVEAEDVGVYYCWQGTHFPYTFGQGTKLEIK

SEQ ID NO: 139 - protein, 112 aa, Synthetic construct
DVVMTQSPLSLPVTLGQPASISCKSSQSLLDSDGKTYLNWLQQRPGQSPRRLIYLVGKDDSGVPDRFSGSGSGTDFTLKISRVEAEDVGVYYCWQGTHFPYTFGQGTKLEIK

SEQ ID NO: 140 - protein, 112 aa, Synthetic construct
DVVMTQSPLSLPVTLGQPASISCKSSQSLLDSDGKTYLNWLQQRPGQSPRRLIYDVSKGDSGVPDRFSGSGSGTDFTLKISRVEAEDVGVYYCWQGTHFPYTFGQGTKLEIK

SEQ ID NO: 141 - protein, 112 aa, Synthetic construct
DVVMTQSPLSLPVTLGQPASISCKSSQSLLDSDGKTYLNWLQQRPGQSPRRLIYDVSKRDSGVPDRFSGSGSGTDFTLKISRVEAEDVGVYYCWQGTHFPYTFGQGTKLEIK

SEQ ID NO: 142 - protein, 112 aa, Synthetic construct
DVVMTQSPLSLPVTLGQPASISCKSSQSLLDSDGKTYLNWLQQRPGQSPRRLIYVVSKDDSGVPDRFSGSGSGTDFTLKISRVEAEDVGVYYCWQGTHFPYTFGQGTKLEIK

SEQ ID NO: 143 - protein, 112 aa, Synthetic construct
DVVMTQSPLSLPVTLGQPASISCKSSQSLLDSDGKTYLNWLQQRPGQSPRRLIYLVSKLDSGVPDRFSGSGSGTDFTLKISRVEAEDVGVYYCWQGTHFPYTFGGGTKLEIK

SEQ ID NO: 144 - protein, 112 aa, Synthetic construct
DVVMTQSPLSLPVTLGQPASISCKSSQSLLDSDGKTYLNWLLQRPGQSPRRLIYLVSKLDSGVPDRFSGSGSGTDFTLKISRVEAEDVGVYYCWQGTHFPYTFGQGTKLEIK

SEQ ID NO: 145 - protein, 112 aa, Synthetic construct
DVVMTQSPLSLPVTLGQPASISCKSSQSLLDSDGKTYLNWLQQRPGQSPRRLIYLVSKEDSGVPDRFSGSGSGTDFTLKISRVEAEDVGVYYCWQGTHFPYTFGGGTKLEIK

SEQ ID NO: 146 - protein, 112 aa, Synthetic construct
EVQLVQSGAEVVKPGATVKISCKASGFNIKDYYLHWVRQRPGQGLEWIGWIDPENGDTVYEPKFQGRATITADTSTDTAYLQLGSLTSEDTAVYFCSTLDFWGQGTLVTVSS

SEQ ID NO: 147 - protein, 112 aa, Synthetic construct
EVQLVQSGAEVVKPGATVKISCKASGFNIKDYYLHWVRQRPGQGLEWIGWIDPENGDTVYDPKFQGRATITADTSTDTAYLQLGSVTSEDTAVYFCSTLDFWGQGTLVTVSS

SEQ ID NO: 148 - protein, 112 aa, Synthetic construct
EVQLVQSGAEVVKPGATVKISCKASGFNIKDYYLHWVRQRPGQGLEWIGWIDPENGDTVYEPKFQGRATITADTSTDTAYMELGSVRSEDTAVYFCSTLDFWGQGTLVTVSS

SEQ ID NO: 149 - protein, 17 aa, Synthetic construct
WIDPENGDTVYEPKFQG

SEQ ID NO: 150 - protein, 7 aa, Synthetic construct
LVSKDDS

SEQ ID NO: 151 - protein, 7 aa, Synthetic construct
LVSKGDS

SEQ ID NO: 152 - protein, 7 aa, Synthetic construct
LVSKNDS

SEQ ID NO: 153 - protein, 7 aa, Synthetic construct
LVSKEDS

SEQ ID NO: 154 - protein, 7 aa, Synthetic construct
EVSKLDS

SEQ ID NO: 155 - protein, 7 aa, Synthetic construct
LVSKQDS

SEQ ID NO: 156 - protein, 7 aa, Synthetic construct
DVSKLDS

SEQ ID NO: 157 - protein, 7 aa, Synthetic construct
LVSKKDS

SEQ ID NO: 158 - protein, 7 aa, Synthetic construct
LVSKRDS

SEQ ID NO: 159 - protein, 7 aa, Synthetic construct
LVSKTDS

SEQ ID NO: 160 - protein, 7 aa, Synthetic construct
GVSKLDS

SEQ ID NO: 161 - protein, 7 aa, Synthetic construct
LVSKVDS

SEQ ID NO: 162 - protein, 7 aa, Synthetic construct
LVSKSDS

SEQ ID NO: 163 - protein, 7 aa, Synthetic construct
LVGKLDS

SEQ ID NO: 164 - protein, 7 aa, Synthetic construct
VVSKLDS

SEQ ID NO: 165 - protein, 7 aa, Synthetic construct
GVSKRDS

SEQ ID NO: 166 - protein, 7 aa, Synthetic construct
GVSKGDS

SEQ ID NO: 167 - protein, 7 aa, Synthetic construct
LVGKGDS

SEQ ID NO: 168 - protein, 7 aa, Synthetic construct
LVGKRDS

SEQ ID NO: 169 - protein, 7 aa, Synthetic construct
LVGKTDS

SEQ ID NO: 170 - protein, 7 aa, Synthetic construct
LVGKDDS

SEQ ID NO: 171 - protein, 7 aa, Synthetic construct
DVSKGDS

SEQ ID NO: 172 - protein, 7 aa, Synthetic construct
DVSKRDS

SEQ ID NO: 173 - protein, 7 aa, Synthetic construct
EVSKGDS

SEQ ID NO: 174 - protein, 7 aa, Synthetic construct
EVSKRDS

SEQ ID NO: 175 - protein, 7 aa, Synthetic construct
VVSKDDS

SEQ ID NO: 176 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 107 aa, Homo sapiens [sequence omitted]

SEQ ID NO: 178 - protein, 442 aa, Synthetic construct
EVQLVQSGAEVVKPGATVKISCKASGFNIKDYYLHWVRQRPGQGLEWIGWIDPENGDTVYDPKFQGRATITADTSTDTAYLQLGSLTSEDTAVYFCSTLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 219 aa, Synthetic construct
DVVMTQSPLSLPVTLGQPASISCKSSQSLLDSDGKTYLNWLQQRPGQSPRRLIYLVGKRDSGVPDRFSGSGSGTDFTLKISRVEAEDVGVYYCWQGTHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 461 aa, Synthetic construct
MMSFVSLLLVGILFHATQAEVQLVQSGAEVVKPGATVKISCKASGFNIKDYYLHWVRQRPGQGLEWIGWIDPENGDTVYDPKFQGRATITADTSTDTAYLQLGSLTSEDTAVYFCSTLDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - protein, 238 aa, Synthetic construct
MMSFVSLLLVGILFHATQADVVMTQSPLSLPVTLGQPASISCKSSQSLLDSDGKTYLNWLQQRPGQSPRRLIYLVGKRDSGVPDRFSGSGSGTDFTLKISRVEAEDVGVYYCWQGTHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - DNA, 1398 bp, Synthetic construct
  source 1..1398: mol_type = other DNA
atgatgtcctttgtctctctgctcctggttggcatcctattccatgccacccaggccgaggtgcagctggtgcagtctggggcagaggttgtgaagccaggggccacagtcaagatctcctgtaaggcttctggcttcaacattaaagactactatctgcactgggtgcggcagaggcctggacagggcctggagtggattggatggattgatcctgagaatggtgatactgtgtatgacccgaagttccagggcagggccactataacagcagacacatccaccgacacagcctacctgcagctcggcagcctgacatctgaggacactgccgtctatttctgttctaccctggacttctggggccaaggcacccttgtcacagtctcctcagcctccaccaagggcccatcggtcttccccctggcaccctctagcaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgagagaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctattccaaactcaccgtggacaagagcaggtggcagcaggggaacgtcttctcatgctccgtgatgcatgaggctctgcacaaccactacacgcagaagagcctctccctgtctcccgggaaatgatgagatctcgag

SEQ ID NO: 183 - DNA, 729 bp, Synthetic construct
  source 1..729: mol_type = other DNA
atgatgtcctttgtctctctgctcctggttggcatcctattccatgccacccaggccgatgttgtgatgacccagtctccactctctttgcccgttacccttggacaacctgcctccatctcttgcaagtcaagtcagagcctcttagatagtgatggaaagacatatttgaattggttgcaacagaggccaggccagtctccacggcgcctaatctatctggtgggcaaacgggactctggagtccctgacaggttcagtggcagtggatcagggacagatttcacactgaaaatcagcagagtggaggctgaggatgtgggagtttattattgctggcaaggcacacattttccgtacacgttcggaggggggaccaagctggaaataaaacgaactgtggctgcaccatctgtcttcatcttcccgccatctgatgagcagcttaagtccggaactgctagcgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtgagatctcgag

SEQ ID NO: 184 - protein, 17 aa, Homo sapiens
NVKSKIGSTENLKHQPG

SEQ ID NO: 185 - protein, 17 aa, Homo sapiens
NVQSKCGSKDNIKHVPG

SEQ ID NO: 186 - protein, 17 aa, Homo sapiens
KVTSKCGSLGNIHHKPG

SEQ ID NO: 187 - protein, 17 aa, Homo sapiens
RVQSKIGSLDNITHVPG

SEQ ID NO: 188 - protein, 10 aa, Homo sapiens
KIGSTENLKH

SEQ ID NO: 189 - protein, 4 aa, Homo sapiens
NVKS

SEQ ID NO: 190 - skipped

SEQ ID NO: 191 - protein, 10 aa, Synthetic construct
  VARIANT 9: note = Xaa = Lys or His
KXXSXXNXXH

SEQ ID NO: 192 - protein, 10 aa, Homo sapiens
KCGSKDNIKH

SEQ ID NO: 193 - protein, 10 aa, Homo sapiens
KCGSLGNIHH

SEQ ID NO: 194 - protein, 10 aa, Homo sapiens
KIGSLDNITH

(19) United States
(12) Patent Application Publication
(10) Pub. No.: US 2024/0018226 A1
(43) Pub. Date: Jan. 18, 2024

(54) METHODS OF USING ANTIBODIES RECOGNIZING TAU

(71) Applicant: Prothena Biosciences Limited, Dublin 2 (IE)

(72) Inventor: Philip James Dolan, III, Oakland, CA (US)

(73) Assignee: Prothena Biosciences Limited, Dublin 2 (IE)

(21) Appl. No.: 18/456,932

(22) Filed: Aug. 28, 2023

Related U.S. Application Data
(63) Continuation of application No. 17/671,420, filed on Feb. 14, 2022, now abandoned.
(60) Provisional application No. 63/149,359, filed on Feb. 14, 2021.

(51) Int. Cl. C07K 16/18; A61P 25/28

(52) U.S. Cl. CPC C07K 16/18; A61P 25/28; C07K 2317/565; C07K 2317/77; A61K 2039/505

(57) ABSTRACT

The invention provides methods of treating taupathies such as Alzheimer's disease with antibodies that bind to human tau. The antibodies inhibit or delay tau-associated pathologies and associated symptomatic deterioration.

### CROSS REFERENCE TO RELATED APPLICATIONS

This application is a continuation of U.S. patent application Ser. No. 17/671,420, filed Feb. 14, 2022, which claims the benefit of U.S. Provisional Patent Application 63/149,359, filed Feb. 14, 2021, each of which is incorporated by reference in its entirety for all purposes. The present application is related to U.S. patent application Ser. No. 16/808,209, filed Mar. 3, 2020, which claims priority to U.S. Provisional Patent Application No. 62/813,126, filed Mar. 3, 2019, U.S. Provisional Patent Application No. 62/813,137, filed Mar. 3, 2019, and U.S. Provisional Patent Application No. 62/838,159, filed Apr. 24, 2019, each of which is incorporated by reference in its entirety for all purposes.

### REFERENCE TO A SEQUENCE LISTING

This application contains a Sequence Listing that has been submitted electronically as an XML file named 50887-0031002 SL ST26.xml. The XML file, created on Aug. 24, 2023, is 185,523 bytes in size. The material in the XML file is hereby incorporated by reference in its entirety.

### BACKGROUND OF THE INVENTION

Tau is a well-known human protein that can exist in phosphorylated forms (see, e.g., Goedert, Proc. Natl. Acad. Sci. U.S.A. 85:4051-4055(1988); Goedert, EMBO J. 8:393-399(1989); Lee, Neuron 2:1615-1624(1989); Goedert, Neuron 3:519-526(1989); Andreadis, Biochemistry 31:10626-10633(1992). Tau has been reported to have a role in stabilizing microtubules, particularly in the central nervous system. Total tau (t-tau, i.e., phosphorylated and unphosphorylated forms) and phospho-tau (p-tau, i.e., phosphorylated tau) are released by the brain in response to neuronal injury and neurodegeneration and have been reported to occur at increased levels in the CSF of Alzheimer's patients relative to the general population (Jack et al., Lancet Neurol 9: 119-28 (2010)).

Tau is the principal constituent of neurofibrillary tangles, which together with plaques are a hallmark characteristic of Alzheimer's disease. The tangles constitute abnormal fibrils measuring 10 nm in diameter occurring in pairs wound in a helical fashion with a regular periodicity of 80 nm. The tau within neurofibrillary tangles is abnormally phosphorylated (hyperphosphorylated) with phosphate groups attached to specific sites on the molecule. Severe involvement of neurofibrillary tangles is seen in the layer II neurons of the entorhinal cortex, the CA1 and subicular regions of the hippocampus, the amygdala, and the deeper layers (layers III, V, and superficial VI) of the neocortex in Alzheimer's disease. Hyperphosphorylated tau has also been reported to interfere with microtubule assembly, which may promote neuronal network breakdown.

Tau inclusions are part of the defining neuropathology of several neurodegenerative diseases including Alzheimer's disease, frontotemporal lobar degeneration, progressive supranuclear palsy and Pick's disease.

### BRIEF SUMMARY OF THE CLAIMED INVENTION

In one aspect, the invention provides a method of reducing internalization of tau by cells in a subject comprising administering to a subject in need thereof an amount of an antibody or an antigen-binding fragment thereof that reduces internalization of tau by cells, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO:14.

In another aspect, the invention provides a method of reducing tau induced toxicity in a subject comprising administering to a subject in need thereof an amount of an antibody or an antigen-binding fragment thereof that reduces tau induced toxicity, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO:14.

In another aspect, the invention provides a method of reducing or delaying onset of behavioral deficit in a subject comprising administering to a subject in need thereof an amount of an antibody or an antigen-binding fragment thereof that reduces or delays onset of behavioral deficit, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO:14.

In another aspect, the invention provides a method of reducing levels of markers of tau pathology in a subject comprising administering to a subject in need thereof an amount of an antibody or an antigen-binding fragment thereof that reduces markers of tau pathology, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO:14.

In another aspect, the invention provides a method of reducing development of tau pathology in a subject comprising administering to a subject in need thereof an amount of an antibody or an antigen-binding fragment thereof that reduces tau pathology, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO:14.

In some methods, the subject has pathological features of Alzheimer's disease. In some methods, the subject has Alzheimer's disease.

In some methods, the CDR-L2 of the antibody or antigen-binding fragment comprises SEQ ID NO:13. In some methods, the CDR-L2 of the antibody or antigen-binding fragment comprises SEQ ID NO:168.

In some methods, the heavy chain variable region of the antibody or antigen-binding fragment comprises a mature heavy chain variable region of SEQ ID NO:18 and the light chain variable region of the antibody or antigen-binding fragment comprises a mature light chain variable region of SEQ ID NO:122. In some methods, the antibody or antigen-binding fragment is a humanized version of a mouse antibody characterized by a mature heavy chain variable region of SEQ ID NO: 7 and a mature light chain variable region of SEQ ID NO:11.

In some methods, the antibody comprises a light chain comprising the mature light chain variable region fused to a light chain constant region and a heavy chain comprising the mature heavy chain variable region fused to a heavy chain constant region.

In some methods, the heavy chain constant region of the antibody comprises the amino acid sequence of SEQ ID NO:176 with or without the C-terminal lysine. In some methods, the mature heavy chain variable region fused to the heavy chain constant region comprises the amino acid sequence of SEQ ID NO:178 with or without the C-terminal lysine.

In some methods, the antibody further comprises a signal peptide fused to the mature heavy and/or light chain variable region. In some methods, the heavy chain comprises the amino acid sequence of SEQ ID NO:180 with or without C-terminal lysine.

In some methods, the light chain constant region of the antibody comprises the amino acid sequence of SEQ ID NO:177. In some methods, the mature light chain variable region fused to a light chain constant region comprises the amino acid sequence of SEQ ID NO:179. In some methods, the light chain comprises the amino acid sequence of SEQ ID NO:181.

In some methods, the heavy chain comprises the amino acid sequence of SEQ ID NO:178 with or without the C-terminal lysine and the light chain comprises the amino acid sequence of SEQ ID NO:179. In some methods, the heavy chain comprises the amino acid sequence of SEQ ID NO:180 with or without the C-terminal lysine and the light chain comprises the amino acid sequence of SEQ ID NO:181.

In some methods, the antibody comprise at least one mutation in the constant region. In some methods, the antibody comprise at least one mutation in the constant region, wherein the mutation reduces complement fixation or activation by the constant region or reduces binding to a Fcγ receptor relative to the natural human heavy chain constant region. In some methods, the antibody comprises a mutation at one or more of positions 241, 264, 265, 270, 296, 297, 318, 320, 322, 329 and 331 by EU numbering or alanine at positions 318, 320 and 322.

### BRIEF DESCRIPTION OF THE DRAWINGS

FIG. 1 shows results of tau internalization assay for mouse 3D6 and hu3D6VHv1bA11/L2-DIM4.

FIGS. 2A and 2B show that mouse 3D6 interrupts tau seeding in an in vivo disease model of Alzheimer's disease.

FIG. 3 shows that mouse 3D6 treatment reduces pathological tau and ameliorates behavior deficit in a transgenic tau model

FIG. 4 shows that mouse 3D6 protects mouse primary cortical neurons from tau-induced toxicity.

### BRIEF DESCRIPTION OF THE SEQUENCES

SEQ ID NO:1 sets forth the amino acid sequence of an isoform of human tau (Swiss-Prot P10636-8).

SEQ ID NO:2 sets forth the amino acid sequence of an isoform of human tau (Swiss-Prot P10636-7).

SEQ ID NO:3 sets forth the amino acid sequence of an isoform of human tau (Swiss-Prot P10636-6), (4RON human tau).

SEQ ID NO:4 sets forth the amino acid sequence of an isoform of human tau (Swiss-Prot P10636-5)

SEQ ID NO:5 sets forth the amino acid sequence of an isoform of human tau (Swiss-Prot P10636-4).

SEQ ID NO:6 sets forth the amino acid sequence of an isoform of human tau (Swiss-Prot P10636-2).

SEQ ID NO:7 sets forth the amino acid sequence of the heavy chain variable region of the mouse 3D6 antibody.

SEQ ID NO:8 sets forth the amino acid sequence of Kabat/Chothia composite CDR-H1 of the mouse 3D6 antibody.

SEQ ID NO:9 sets forth the amino acid sequence of Kabat CDR-H2 of the mouse 3D6 antibody.

SEQ ID NO:10 sets forth the amino acid sequence of Kabat CDR-H3 of the mouse 3D6 antibody.

SEQ ID NO:11 sets forth the amino acid sequence of the light chain variable region of the mouse 3D6 antibody and of the mouse 6A10 antibody.

SEQ ID NO:12 sets forth the amino acid sequence of Kabat CDR-L1 of the mouse 3D6 antibody and of the mouse 6A10 antibody.

SEQ ID NO:13 sets forth the amino acid sequence of Kabat CDR-L2 of the mouse 3D6 antibody and of the mouse 6A10 antibody.

SEQ ID NO:14 sets forth the amino acid sequence of Kabat CDR-L3 of the mouse 3D6 antibody and of the mouse 6A10 antibody.

SEQ ID NO:15 sets forth the amino acid sequence of heavy chain variable region of the humanized 3D6 antibody hu3D6VHv1.

SEQ ID NO:16 sets forth the amino acid sequence of heavy chain variable region of the humanized 3D6 antibody hu3D6VHv2.

SEQ ID NO:17 sets forth the amino acid sequence of heavy chain variable region of the humanized 3D6 antibody hu3D6VHv1b.

SEQ ID NO:18 sets forth the amino acid sequence of heavy chain variable region of the humanized 3D6 antibody hu3D6VHv1bA11.

SEQ ID NO:19 sets forth the amino acid sequence of heavy chain variable region of the humanized 3D6 antibody hu3D6VHv5:

SEQ ID NO:20 sets forth the amino acid sequence of the light chain variable region of the humanized 3D6 antibody hu3D6VLv1.

SEQ ID NO:21 sets forth the amino acid sequence of the light chain variable region of the humanized 3D6 antibody hu3D6VLv2.

SEQ ID NO:22 sets forth the amino acid sequence of the light chain variable region of the humanized 3D6 antibody hu3D6VLv3.

SEQ ID NO:23 sets forth the amino acid sequence of the light chain variable region of the humanized 3D6 antibody hu3D6VLv4.

SEQ ID NO:24 sets forth the amino acid sequence of the heavy chain variable acceptor Acc. #BAC01986.1.

SEQ ID NO:25 sets forth the amino acid sequence of the heavy chain variable acceptor Acc. #IMGT #IGHV1-69-2*01.

SEQ ID NO:26 sets forth the amino acid sequence of the heavy chain variable acceptor Acc. #IMGT #IGKJ1*01.

SEQ ID NO:27 sets forth the amino acid sequence of the light chain variable acceptor Acc. #IMGT #IGKV2-30*02

SEQ ID NO:28 sets forth the amino acid sequence of the light chain variable acceptor Acc. #IMGT #IGKJ2*01.

SEQ ID NO:29 sets forth the amino acid sequence of the light chain variable acceptor Acc. #AAZ09048.1.

SEQ ID NO:30 sets forth a nucleic acid sequence encoding the heavy chain variable region of the mouse 3D6 antibody.

SEQ ID NO:31 sets forth a nucleic acid sequence encoding the light chain variable region of the mouse 3D6 antibody.

SEQ ID NO:32 sets forth the amino acid sequence of Kabat CDR-H1 of the mouse 3D6 antibody.

SEQ ID NO:33 sets forth the amino acid sequence of Chothia CDR-H1 of the mouse 3D6 antibody.

SEQ ID NO:34 sets forth the amino acid sequence of Chothia CDR-H2 of the mouse 3D6 antibody.

SEQ ID NO:35 sets forth the amino acid sequence of AbM CDR-H2 of the mouse 3D6 antibody.

SEQ ID NO:36 sets forth the amino acid sequence of Contact CDR-L1 of the mouse 3D6 antibody.

SEQ ID NO:37 sets forth the amino acid sequence of Contact CDR-L2 of the mouse 3D6 antibody.

SEQ ID NO:38 sets forth the amino acid sequence of Contact CDR-L3 of the mouse 3D6 antibody.

SEQ ID NO:39 sets forth the amino acid sequence of Contact CDR-H1 of the mouse 3D6 antibody.

SEQ ID NO:40 sets forth the amino acid sequence of Contact CDR-H2 of the mouse 3D6 antibody.

SEQ ID NO:41 sets forth the amino acid sequence of Contact CDR-H3 of the mouse 3D6 antibody.

SEQ ID NO:42 sets forth the amino acid sequence of an alternate Kabat-Chothia Composite CDR-H1 of a humanized 3D6 antibody (as in hu3D6VHv5, hu3D6VHv1bA11B6G2, hu3D6VHv1bA11B6H3, hu3D6VHv1e, and hu3D6VHv1f).

SEQ ID NO:43 sets forth the amino acid sequence of an alternate Kabat CDR-H2 of a humanized 3D6 antibody (as in hu3D6VHv5 and hu3D6VHv1bA11B6H3).

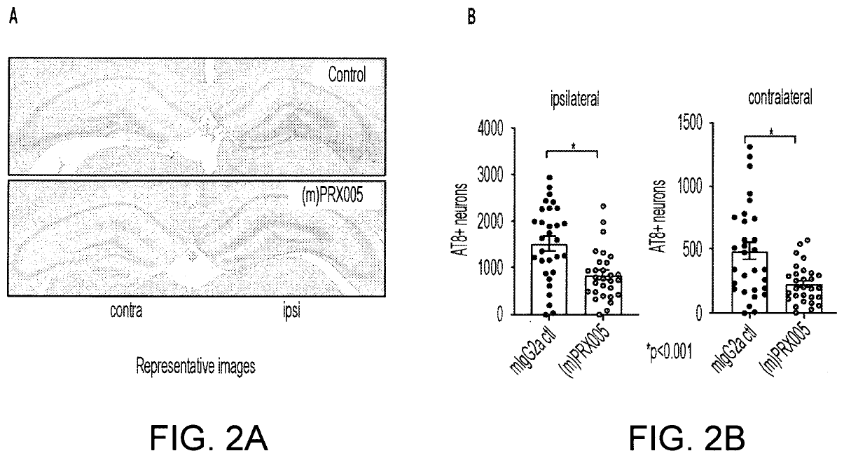

SEQ ID NO:44 sets forth the consensus amino acid sequence among the heavy chain variable regions of the mouse 3D6 and selected humanized 3D6 antibodies (VHv1, VHv2, VHv1b, VHv1bA11, and VHv5) (labeled “Majority’ in FIG. 2 of PCT/IB2017/052544.

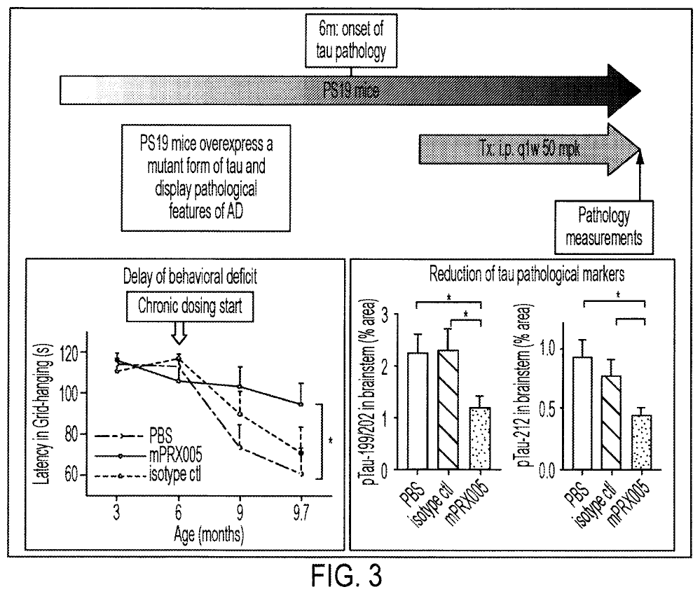

SEQ ID NO:45 sets forth the consensus amino acid sequence between the light chain variable regions of the mouse 3D6 and selected humanized 3D6 antibodies (labeled “Majority’ in FIG. 3 of PCT/IB2017/052544).

SEQ ID NO:46 sets forth the amino acid sequence of heavy chain variable region of the humanized 3D6 antibody hu3D6VHv1bA11B6G2.

SEQ ID NO:47 sets forth the amino acid sequence of heavy chain variable region of the humanized 3D6 antibody hu3D6VHv1bA11B6H3.

SEQ ID NO:48 sets forth the amino acid sequence of heavy chain variable region of the humanized 3D6 antibody hu3D6VHv1c.

SEQ ID NO:49 sets forth the amino acid sequence of heavy chain variable region of the humanized 3D6 antibody hu3D6VHv1d.

SEQ ID NO:50 sets forth the amino acid sequence of heavy chain variable region of the humanized 3D6 antibody hu3D6VHv1e.

SEQ ID NO:51 sets forth the amino acid sequence of heavy chain variable region of the humanized 3D6 antibody hu3D6VHv1f.

SEQ ID NO:52 sets forth the amino acid sequence of heavy chain variable region of the humanized 3D6 antibody hu3D6VHv3.

SEQ ID NO:53 sets forth the amino acid sequence of heavy chain variable region of the humanized 3D6 antibody hu3D6VHv3b.

SEQ ID NO:54 sets forth the amino acid sequence of heavy chain variable region of the humanized 3D6 antibody hu3D6VHv3c.

SEQ ID NO:55 sets forth the amino acid sequence of heavy chain variable region of the humanized 3D6 antibody hu3D6VHv4.

SEQ ID NO:56 sets forth the amino acid sequence of heavy chain variable region of the humanized 3D6 antibody hu3D6VHv4b.

SEQ ID NO:57 sets forth the amino acid sequence of heavy chain variable region of the humanized 3D6 antibody hu3D6VHv4c.

SEQ ID NO:58 sets forth the amino acid sequence of an alternate Kabat-Chothia Composite CDR-H1 of a humanized 3D6 antibody (as in hu3D6VH1c).

SEQ ID NO:59 sets forth the amino acid sequence of an alternate Kabat-Chothia Composite CDR-H1 of a humanized 3D6 antibody (as in hu3D6VHv1d, hu3D6VHv3c, and hu3D6VHv4c).

SEQ ID NO:60 sets forth the amino acid sequence of an alternate Kabat-Chothia Composite CDR-H1 of a humanized 3D6 antibody (as in hu3D6VHv3b and hu3D6VHv4b).

SEQ ID NO:61 sets forth the amino acid sequence of an alternate Kabat CDR-H2 of a humanized 3D6 antibody (as in hu3D6VHv1bA11B6G2).

SEQ ID NO:62 sets forth the amino acid sequence of an alternate Kabat CDR-H2 of a humanized 3D6 antibody (as in hu3D6VHv1c, hu3D6VHv3b, AND hu3D6VHv4b.

SEQ ID NO:63 sets forth the amino acid sequence of an alternate Kabat CDR-H2 of a humanized 3D6 antibody (as in hu3D6VHv1d, hu3D6VHv1f, hu3D6VHv3c, and hu3D6VHv4c).

SEQ ID NO:64 sets forth the amino acid sequence of an alternate Kabat CDR-H2 of a humanized 3D6 antibody (as in hu3D6VHv1e).

SEQ ID NO:65 sets forth the amino acid sequence of an alternate Kabat CDR-H3 of a humanized 3D6 antibody (as in hu3D6VHv1f).

SEQ ID NO:66 sets forth the amino acid sequence of the heavy chain variable region of the mouse 6A10 antibody.

SEQ ID NO:67 sets forth the amino acid sequence of Kabat/Chothia composite CDR-H1 of the mouse 6A10 antibody.

SEQ ID NO:68 sets forth the amino acid sequence of Kabat CDR-H2 of the mouse 6A10 antibody.

SEQ ID NO:69 sets forth the amino acid sequence of Kabat CDR-H3 of the mouse 6A10 antibody.

SEQ ID NO:70 sets for the amino acid sequence of the VH region of mouse antibody (pdb code 1CR9) used as a structure template for heavy chain humanization.

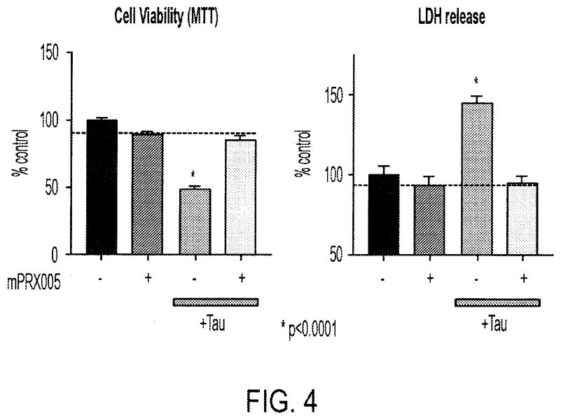

SEQ ID NO:71 sets forth the consensus amino acid sequence among the heavy chain variable regions of the selected humanized 3D6 antibodies (VHv1, VHv1b, VHv1bA11, VHv1bA11B6G2, VHv1bA11B6H3, VHv1c, VHv1d, VHv1e, VHv1f, VHv2, VHv3, VHv3b, VHv3c, VHv4, VHv4b, VHv4c, and VHv5) (labeled “Majority’ in FIGS. 4A and 4B of PCT/IB2017/052544).

SEQ ID NO:72 sets forth the amino acid sequence of the heavy chain of a chimeric 3D6 antibody.

SEQ ID NO:73 sets forth the amino acid sequence of the light chain of a chimeric 3D6 antibody.

SEQ ID NO:74 sets forth the amino acid sequence of heavy chain variable structural model Acc. #5MYX-VH_mSt.

SEQ ID NO:75 sets forth the amino acid sequence of heavy chain variable acceptor Acc. #2RCS-VH_huFrwk.

SEQ ID NO:76 sets forth the amino acid sequence of heavy chain variable region of the humanized 3D6 antibody hu3D6VHvb1.

SEQ ID NO:77 sets forth the amino acid sequence of heavy chain variable region of the humanized 3D6 antibody hu3D6VHvb2.

SEQ ID NO:78 sets forth the amino acid sequence of heavy chain variable region of the humanized 3D6 antibody hu3D6VHvb3.

SEQ ID NO:79 sets forth the amino acid sequence of heavy chain variable region of the humanized 3D6 antibody hu3D6VHvb4.

SEQ ID NO:80 sets forth the amino acid sequence of heavy chain variable region of the humanized 3D6 antibody hu3D6VHvb5.

SEQ ID NO:81 sets forth the amino acid sequence of light chain variable structural model Acc. #5MYX-VL_mSt.

SEQ ID NO:82 sets forth the amino acid sequence of light chain variable acceptor Acc. #ARX71335-VL_huFrwk.

SEQ ID NO:83 sets forth the amino acid sequence of light chain variable region of the humanized 3D6 antibody hu3D6VLvb1.

SEQ ID NO:84 sets forth the amino acid sequence of light chain variable region of the humanized 3D6 antibody hu3D6VLvb2.

SEQ ID NO:85 sets forth the amino acid sequence of light chain variable region of the humanized 3D6 antibody hu3D6VLvb3.

SEQ ID NO:86 sets forth the amino acid sequence of an alternate Kabat-Chothia Composite CDR-H1 of a humanized 3D6 antibody (as in hu3D6VHvb4 and hu3D6VHvb5).

SEQ ID NO:87 sets forth the amino acid sequence of an alternate Kabat CDR-H2 of a humanized 3D6 antibody (as in hu3D6VHvb3 and hu3D6VHvb4).

SEQ ID NO:88 sets forth the amino acid sequence of an alternate Kabat CDR-H2 of a humanized 3D6 antibody (as in hu3D6VHvb5).

SEQ ID NO:89 sets forth the amino acid sequence of an alternate Kabat CDR-L1 of a humanized 3D6 antibody (as in hu3D6VLvb3).

SEQ ID NO:90 sets forth the amino acid sequence of heavy chain variable region of the humanized 3D6 antibody hu3D6VHvb6.

SEQ ID NO:91 sets forth the amino acid sequence of heavy chain variable region of the humanized 3D6 antibody hu3D6VHvb7.

SEQ ID NO:92 sets forth the amino acid sequence of an alternate Kabat CDR-H2 of a humanized 3D6 antibody (as in hu3D6VHvb6 and hu3D6VHvb7).

SEQ ID NO:93 sets forth the amino acid sequence of light chain variable region of a hu3D6VLv2 variant L54D.

SEQ ID NO:94 sets forth the amino acid sequence of light chain variable region of a hu3D6VLv2 variant L54G.

SEQ ID NO:95 sets forth the amino acid sequence of light chain variable region of a hu3D6VLv2 variant L45N.

SEQ ID NO:96 sets forth the amino acid sequence of light chain variable region of a hu3D6VLv2 variant L54E.

SEQ ID NO:97 sets forth the amino acid sequence of light chain variable region of a hu3D6VLv2 variant L50E.

SEQ ID NO:98 sets forth the amino acid sequence of light chain variable region of a hu3D6VLv2 variant L54Q.

SEQ ID NO:99 sets forth the amino acid sequence of light chain variable region of a hu3D6VLv2 variant L50D.

SEQ ID NO:100 sets forth the amino acid sequence of light chain variable region of a hu3D6VLv2 variant L54K.

SEQ ID NO:101 sets forth the amino acid sequence of light chain variable region of a hu3D6VLv2 variant L54R.

SEQ ID NO:102 sets forth the amino acid sequence of light chain variable region of a hu3D6VLv2 variant L54T.

SEQ ID NO:103 sets forth the amino acid sequence of light chain variable region of a hu3D6VLv2 variant L50G.

SEQ ID NO:104 sets forth the amino acid sequence of light chain variable region of a hu3D6VLv2 variant 148G.

SEQ ID NO:105 sets forth the amino acid sequence of light chain variable region of a hu3D6VLv2 variant I48D.

SEQ ID NO:106 sets forth the amino acid sequence of light chain variable region of a hu3D6VLv2 variant L47G.

SEQ ID NO:107 sets forth the amino acid sequence of light chain variable region of a hu3D6VLv2 variant Y49E.

SEQ ID NO:108 sets forth the amino acid sequence of light chain variable region of a hu3D6VLv2 variant L54V.

SEQ ID NO:109 sets forth the amino acid sequence of light chain variable region of a hu3D6VLv2 variant L54S.

SEQ ID NO:110 sets forth the amino acid sequence of light chain variable region of a hu3D6VLv2 variant S52G.

SEQ ID NO:111 sets forth the amino acid sequence of light chain variable region of a hu3D6VLv2 variant L47N.

SEQ ID NO:112 sets forth the amino acid sequence of light chain variable region of a hu3D6VLv2 variant L47D.

SEQ ID NO:113 sets forth the amino acid sequence of light chain variable region of a hu3D6VLv2 variant L47E.

SEQ ID NO:114 sets forth the amino acid sequence of light chain variable region of a hu3D6VLv2 variant L47P.

SEQ ID NO:115 sets forth the amino acid sequence of light chain variable region of a hu3D6VLv2 variant L47T.

SEQ ID NO:116 sets forth the amino acid sequence of light chain variable region of a hu3D6VLv2 variant L47S.

SEQ ID NO:117 sets forth the amino acid sequence of light chain variable region of a hu3D6VLv2 variant L47A.

SEQ ID NO:118 sets forth the amino acid sequence of light chain variable region of a hu3D6VLv2 variant L50V.

SEQ ID NO:119 sets forth the amino acid sequence of light chain variable region of a hu3D6VLv2 variant L37Q_L50G_L54R.

SEQ ID NO:120 sets forth the amino acid sequence of light chain variable region of a hu3D6VLv2 variant L37QL50G_L54G.

SEQ ID NO:121 sets forth the amino acid sequence of light chain variable region of a hu3D6VLv2 variant L37Q_S52G_L54G.

SEQ ID NO:122 sets forth the amino acid sequence of light chain variable region of a hu3D6VLv2 variant L37Q_S52G_L54R.

SEQ ID NO:123 sets forth the amino acid sequence of light chain variable region of a hu3D6VLv2 variant L37QS52G_L54T.

SEQ ID NO:124 sets forth the amino acid sequence of light chain variable region of a hu3D6VLv2 variant L37Q_S52G_L54D.

SEQ ID NO:125 sets forth the amino acid sequence of light chain variable region of a hu3D6VLv2 variant L37Q_L54R.

SEQ ID NO:126 sets forth the amino acid sequence of light chain variable region of a hu3D6VLv2 variant L37Q_L54G.

SEQ ID NO:127 sets forth the amino acid sequence of light chain variable region of a hu3D6VLv2 variant L37Q_L54D.

SEQ ID NO:128 sets forth the amino acid sequence of light chain variable region of a hu3D6VLv2 variant L37Q_L50D.

SEQ ID NO:129 sets forth the amino acid sequence of light chain variable region of a hu3D6VLv2 variant L37Q_L50D.

SEQ ID NO:130 sets forth the amino acid sequence of light chain variable region of a hu3D6VLv2 variant L37Q_L54T.

SEQ ID NO:131 sets forth the amino acid sequence of light chain variable region of a hu3D6VLv2 variant L37Q_S52T.

SEQ ID NO:132 sets forth the amino acid sequence of light chain variable region of a hu3D6VLv2 variant L37Q_L50D_L54G.

SEQ ID NO:133 sets forth the amino acid sequence of light chain variable region of a hu3D6VLv2 variant L37Q_L50D_L54R.

SEQ ID NO:134 sets forth the amino acid sequence of light chain variable region of a hu3D6VLv2 variant L37Q_L50E_L54G.

SEQ ID NO:135 sets forth the amino acid sequence of light chain variable region of a hu3D6VLv2 variant L37Q_L50E_L54R.

SEQ ID NO:136 sets forth the amino acid sequence of light chain variable region of a hu3D6VLv2 variant L37Q_L50G_L54R_G100Q.

SEQ ID NO:137 sets forth the amino acid sequence of light chain variable region of a hu3D6VLv2 variant L37Q_L50G_L54G_G100Q.

SEQ ID NO:138 sets forth the amino acid sequence of light chain variable region of a hu3D6VLv2 variant L37Q_S52G_L54R_G100Q.

SEQ ID NO:139 sets forth the amino acid sequence of light chain variable region of a hu3D6VLv2 variant L37Q_S52G_L54D_G100Q.

SEQ ID NO:140 sets forth the amino acid sequence of light chain variable region of a Hu3D6VLv2 variant L37Q_L50D_L54G_G100Q.

SEQ ID NO:141 sets forth the amino acid sequence of light chain variable region of a Hu3D6VLv2 variant L37Q_L50D_L54R_G100Q.

SEQ ID NO: 142 sets forth the amino acid sequence of light chain variable region of a Hu3D6VLv2 variant L37Q_L50V_L54D_G100Q.

SEQ ID NO: 143 sets forth the amino acid sequence of light chain variable region of a Hu3D6VLv2 variant L37Q.

SEQ ID NO:144 sets forth the amino acid sequence of light chain variable region of a Hu3D6VLv2 variant G100Q.

SEQ ID NO:145 sets forth the amino acid sequence of light chain variable region of a hu3D6VLv2 variant L37Q_L54E.

SEQ ID NO:146 sets forth the amino acid sequence of heavy chain variable region of a hu3D6VHv1bA11 variant D60E, also known as h3D6VHvb8.

SEQ ID NO: 147 sets forth the amino acid sequence of heavy chain variable region of a hu3D6VHv1bA11 variant L82cV.

SEQ ID NO:148 sets forth the amino acid sequence of heavy chain variable region of a hu3D6VHv1bA11 variant D60E_L80M_Q8IE L82cV_T83R, also known as h3D6VHvb9.

SEQ ID NO:149 sets forth the amino acid sequence of an alternate Kabat CDR-H2 of a humanized 3D6 antibody (as in h3D6VHvb8 and in h3D6VHvb9).

SEQ ID NO:150 sets forth the amino acid sequence of an alternate Kabat CDR-L2 of a humanized 3D6 antibody (as in hu3D6VLv2 L54D and in hu3D6VLv2 L37Q_L54D).

SEQ ID NO:151 sets forth the amino acid sequence of an alternate Kabat CDR-L2 of a humanized 3D6 antibody (as in hu3D6VLv2 L54G and in hu3D6VLv2 L37Q_L54G).

SEQ ID NO:152 sets forth the amino acid sequence of an alternate Kabat CDR-L2 of a humanized 3D6 antibody (as in hu3D6VLv2 L54N).

SEQ ID NO:153 sets forth the amino acid sequence of an alternate Kabat CDR-L2 of a humanized 3D6 antibody (as in hu3D6VLv2 L54E and in hu3D6VLv2 L37Q_L54E).

SEQ ID NO:154 sets forth the amino acid sequence of an alternate Kabat CDR-L2 of a humanized 3D6 antibody (as in hu3D6VLv2 L50E).

SEQ ID NO:155 sets forth the amino acid sequence of an alternate Kabat CDR-L2 of a humanized 3D6 antibody (as in hu3D6VLv2 L54Q).

SEQ ID NO:156 sets forth the amino acid sequence of an alternate Kabat CDR-L2 of a humanized 3D6 antibody (as in hu3D6VLv2 L50D and in hu3D6VLv2 L37Q_L50D).

SEQ ID NO:157 sets forth the amino acid sequence of an alternate Kabat CDR-L2 of a humanized 3D6 antibody (as in hu3D6VLv2 L54K).

SEQ ID NO:158 sets forth the amino acid sequence of an alternate Kabat CDR-L2 of a humanized 3D6 antibody (as in hu3D6VLv2 L54R and in hu3D6VLv2 L37Q_L54R).

SEQ ID NO:159 sets forth the amino acid sequence of an alternate Kabat CDR-L2 of a humanized 3D6 antibody (as in hu3D6VLv2 L54T and in hu3D6VLv2 L37Q_L54T).

SEQ ID NO:160 sets forth the amino acid sequence of an alternate Kabat CDR-L2 of a humanized 3D6 antibody (as in hu3D6VLv2 L50G and in hu3D6VLv2 L37Q_L50G).

SEQ ID NO:161 sets forth the amino acid sequence of an alternate Kabat CDR-L2 of a humanized 3D6 antibody (as in hu3D6VLv2 L54V).

SEQ ID NO:162 sets forth the amino acid sequence of an alternate Kabat CDR-L2 of a humanized 3D6 antibody (as in hu3D6VLv2 L54S).

SEQ ID NO:163 sets forth the amino acid sequence of an alternate Kabat CDR-L2 of a humanized 3D6 antibody (as in hu3D6VLv2 S52G and in hu3D6VLv2 L37Q_S52G).

SEQ ID NO:164 sets forth the amino acid sequence of an alternate Kabat CDR-L2 of a humanized 3D6 antibody (as in hu3D6VLv2 L50V).

SEQ ID NO:165 sets forth the amino acid sequence of an alternate Kabat CDR-L2 of a humanized 3D6 antibody (as in hu3D6VLv2 L37Q_L50G_L54R and hu3D6VLv2 L37Q_L50G_L54R_G100Q).

SEQ ID NO:166 sets forth the amino acid sequence of an alternate Kabat CDR-L2 of a humanized 3D6 antibody (as in hu3D6VLv2 L37Q_L50G_L54G and in and in hu3D6VLv2 L37Q_L50G_L54G_G100Q).

SEQ ID NO:167 sets forth the amino acid sequence of an alternate Kabat CDR-L2 of a humanized 3D6 antibody (as in hu3D6VLv2 L37Q_S52G_L54G).

SEQ ID NO:168 sets forth the amino acid sequence of an alternate Kabat CDR-L2 of a humanized 3D6 antibody (as in hu3D6VLv2 L37Q_S52G_L54R and in and in hu3D6VLv2 L37Q_S52G_L54R_G100Q).

SEQ ID NO:169 sets forth the amino acid sequence of an alternate Kabat CDR-L2 of a humanized 3D6 antibody (as in hu3D6VLv2 L37Q_S52G_L54T).

SEQ ID NO:170 sets forth the amino acid sequence of an alternate Kabat CDR-L2 of a humanized 3D6 antibody (as in hu3D6VLv2 L37Q_S52G_L54D and in hu3D6VLv2 L37Q_S52G_L54D_G100Q).

SEQ ID NO:171 sets forth the amino acid sequence of an alternate Kabat CDR-L2 of a humanized 3D6 antibody (as in hu3D6VLv2 L37Q_L50D_L54G and in hu3D6VLv2 L37Q_L50D_L54G_G100Q).

SEQ ID NO:172 sets forth the amino acid sequence of an alternate Kabat CDR-L2 of a humanized 3D6 antibody (as in hu3D6VLv2 L37Q_L50D_L54R and in hu3D6VLv2 L37Q_L50D_L54R_G100Q).

SEQ ID NO:173 sets forth the amino acid sequence of an alternate Kabat CDR-L2 of a humanized 3D6 antibody (as in hu3D6VLv2 L37Q_L50E_L54G).

SEQ ID NO:174 sets forth the amino acid sequence of an alternate Kabat CDR-L2 of a humanized 3D6 antibody (as in hu3D6VLv2 L37Q_L50E_L54R).

SEQ ID NO:175 sets forth the amino acid sequence of an alternate Kabat CDR-L2 of a humanized 3D6 antibody (as in hu3D6VLv2 L37Q_L50V_L54D_G100Q).

SEQ ID NO:176 sets forth the amino acid sequence of a heavy chain constant region (IgG1: allotype G1m17,1).

SEQ ID NO:177 sets forth the amino acid sequence of a light chain constant region (kappa).

SEQ ID NO:178 sets forth the amino acid sequence of a mature heavy chain of a 3D6 humanized variant (hu3D6VHv1bA11 IgG1 G1m17 allotype).

SEQ ID NO:179 sets forth the amino acid sequence of a mature light chain of a 3D6 humanized variant (hu3D6VLv2 variant L37Q_S52G_L54R, L2-DIM4 kappa).

SEQ ID NO:180 sets forth the amino acid sequence of a heavy chain of a 3D6 humanized variant (hu3D6VHv1bA11 IgG1 G1m17 allotype) with bovine alpha-lactalbumin signal peptide at the N-terminus.

SEQ ID NO:181 sets forth the amino acid sequence of a light chain of a 3D6 humanized variant (hu3D6VLv2 variant L37Q_S52G_L54R, L2-DIM4 kappa) with bovine alpha-lactalbumin signal peptide at the N-terminus.

SEQ ID NO:182 sets forth the nucleotide sequence encoding a heavy chain of a 3D6 humanized variant (hu3D6VHv1bA11 IgG1 G1m17 allotype) with bovine alpha-lactalbumin signal peptide at the N-terminus.

SEQ ID NO:183 sets forth the nucleotide sequence encoding a light chain of a 3D6 humanized variant (hu3D6VLv2 variant L37Q_S52G_L54R, L2-DIM4 kappa) with bovine alpha-lactalbumin signal peptide at the N-terminus.

SEQ ID NO:184 sets forth the amino acid sequence of a region of tau microtubule binding repeat 1 (amino acid residues 255-271 of SEQ ID NO:1).

SEQ ID NO:185 sets forth the amino acid sequence of a region of tau microtubule binding repeat 2 (amino acid residues 286-302 of SEQ ID NO:1).

SEQ ID NO:186 sets forth the amino acid sequence of a region of tau microtubule binding repeat 3 (amino acid residues 317-333 of SEQ ID NO:1).

SEQ ID NO:187 sets forth the amino acid sequence of a region of tau microtubule binding repeat 4 (amino acid residues 349-365 of SEQ ID NO:1).

SEQ ID NO:188 sets forth the amino acid sequence of a core motif of tau in MBTR 1 bound by 3D6.

SEQ ID NO:189 sets forth the amino acid sequence of tau sequence N-terminal to core motif of tau in MBTR 1 bound by 3D6.

SEQ ID NO:190 sets forth the amino acid sequence of tau sequence C-terminal to core motif of tau in MBTR 1 bound by 3D6.

SEQ ID NO:191 sets forth the amino acid sequence of epitope of 3D6.

SEQ ID NO:192 sets forth the amino acid sequence of a core motif of tau in MBTR 2 bound by 3D6.

SEQ ID NO:193 sets forth the amino acid sequence of a core motif of tau in MBTR 3 bound by 3D6.

SEQ ID NO:194 sets forth the amino acid sequence of a core motif of tau in MBTR 4 bound by 3D6.

### Definitions

Monoclonal antibodies or other biological entities are typically provided in isolated form. This means that an antibody or other biologically entity is typically at least 50% w/w pure of interfering proteins and other contaminants arising from its production or purification but does not exclude the possibility that the monoclonal antibody is combined with an excess of pharmaceutically acceptable carrier(s) or other vehicle intended to facilitate its use. Sometimes monoclonal antibodies are at least 60%, 70%, 80%, 90%, 95% or 99% w/w pure of interfering proteins and contaminants from production or purification. Often an isolated monoclonal antibody or other biological entity is the predominant macromolecular species remaining after its purification.

Specific binding of an antibody to its target antigen means an affinity and/or avidity of at least 10⁶, 10⁷, 10⁸, 10⁹, 10¹⁰, 10¹¹, or 10¹² M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that an antibody binds one and only one target.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. See generally, Fundamental Immunology, Paul, W., ed., 2nd ed. Raven Press, N.Y., 1989, Ch. 7 (incorporated by reference in its entirety for all purposes).

An immunoglobulin light or heavy chain variable region (also referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, and FR4. CDRs 1, 2, and 3 of a VL domain are also referred to herein, respectively, as CDR-L1, CDR-L2, and CDR-L3; CDRs 1, 2, and 3 of a VH domain are also referred to herein, respectively, as CDR-H1, CDR-H2, and CDR-H3. When the application discloses a VL sequence with R as the C-terminal residue, the R can alternatively be considered as being the N-terminal residue of the light chain constant region. Thus, the application should also be understood as disclosing the VL sequence without the C-terminal R.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, M D, 1987 and 1991), the Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs; the AbM definition used by Oxford Molecular's antibody modelling software; and, the contact definition of Martin et al (bioinfo.org.uk/abs) (see Table 1). Kabat provides a widely used numbering convention (Kabat numbering) in which corresponding residues between different heavy chains or between different light chains are assigned the same number. When an antibody is said to comprise CDRs by a certain definition of CDRs (e.g., Kabat) that definition specifies the minimum number of CDR residues present in the antibody (i.e., the Kabat CDRs). It does not exclude that other residues falling within another conventional CDR definition but outside the specified definition are also present. For example, an antibody comprising CDRs defined by Kabat includes among other possibilities, an antibody in which the CDRs contain Kabat CDR residues and no other CDR residues, and an antibody in which CDR H1 is a composite Chothia-Kabat CDR H1 and other CDRs contain Kabat CDR residues and no additional CDR residues based on other definitions.

TABLE 1
Conventional Definitions of CDRs Using Kabat Numbering

| | | | Composite of | | |
|---|---|---|---|---|---|
| | | | Chothia & | | |
| Loop | Kabat | Chothia | Kabat | AbM | Contact |
| L1 | L24--L34 | L24--L34 | L24--L34 | L24--L34 | L30--L36 |
| L2 | L50--L56 | L50--L56 | L50--L56 | L50--L56 | L46--L55 |
| L3 | L89--L97 | L89--L97 | L89--L97 | L89--L97 | L89--L96 |
| H1 | H31--H35B | H26--H32 . . . H34* | H26--H35B* | H26--H35B | H30--H35B |
| H2 | H50--H65 | H52--H56 | H50--H65 | H50--H58 | H47--H58 |
| H3 | H95--H102 | H95--H102 | H95--H102 | H95--H102 | H93--H101 |

*CDR-H1 by Chothia can end at H32, H33, or H34 (depending on the length of the loop). This is because the Kabat numbering scheme places insertions of extra residues at 35A and 35B, whereas Chothia numbering places them at 31A and 31B. If neither H35A nor H35B (Kabat numbering) is present, the Chothia CDR-H1 loop ends at H32. If only H35A is present, it ends at H33. If both H35A and H35B are present, it ends at H34.

The term “antibody” includes intact antibodies and binding fragments thereof. Typically, fragments compete with the intact antibody from which they were derived for specific binding to the target including separate heavy chains, light chains Fab, Fab′, F(ab′)2, F(ab)c, Dabs, nanobodies, and Fv. Fragments can be produced by recombinant DNA techniques, or by enzymatic or chemical separation of intact immunoglobulins. The term “antibody” also includes a bispecific antibody and/or a humanized antibody. A bispecific or bifunctional antibody is an artificial hybrid antibody having two different heavy/light chain pairs and two different binding sites (see, e.g., Songsivilai and Lachmann, Clin. Exp. Immunol., 79:315-321 (1990); Kostelny et al., J. Immunol., 148:1547-53 (1992)). In some bispecific antibodies, the two different heavy/light chain pairs include a humanized 3D6 heavy chain/light chain pair and a heavy chain/light chain pair specific for a different epitope on tau than that bound by 3D6.

In some bispecific antibodies, one heavy chain/light chain pair is a humanized 3D6 antibody as further disclosed below and the other heavy chain/light chain pair is from an antibody that binds to a receptor expressed on the blood brain barrier, such as an insulin receptor, an insulin-like growth factor (IGF) receptor, a leptin receptor, or a lipoprotein receptor, or a transferrin receptor (Friden et al., Proc. Natl. Acad. Sci. USA 88:4771-4775, 1991; Friden et al., Science 259:373-377, 1993). Such a bispecific antibody can be transferred cross the blood brain barrier by receptor-mediated transcytosis. Brain uptake of the bispecific antibody can be further enhanced by engineering the bi-specific antibody to reduce its affinity to the blood brain barrier receptor. Reduced affinity for the receptor resulted in a broader distribution in the brain (see, e.g., Atwal et al., Sci. Trans. Med. 3, 84ra43, 2011; Yu et al., Sci. Trans. Med. 3, 84ra44, 2011).

Exemplary bispecific antibodies can also be: (1) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (2) a Tandab, which is a fusion of two single chain diabodies resulting in a tetravalent bispecific antibody that has two binding sites for each of the target antigens; (3) a flexibody, which is a combination of scFvs with a diabody resulting in a multivalent molecule; (4) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; or (5) a so-called Scorpion molecule, comprising, e.g., two scFvs fused to both termini of a human Fc-region. Examples of platforms useful for preparing bispecific antibodies include BiTE (Micromet), DART (MacroGenics), Fcab and Mab2 (F-star), Fc-engineered IgG1 (Xencor) or DuoBody (based on Fab arm exchange, Genmab).

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same or overlapping epitopes can be identified in a simple immunoassay showing the ability of one antibody to compete with the binding of another antibody to a target antigen. The epitope of an antibody can also be defined X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20× or 100×) inhibits binding of the reference antibody by at least 50% as measured in a competitive binding assay. Some test antibodies inhibit binding of the references antibody by at least 75%, 90% or 99%. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “pharmaceutically acceptable” means that the carrier, diluent, excipient, or auxiliary is compatible with the other ingredients of the formulation and not substantially deleterious to the recipient thereof.

The term “patient” includes human and other mammalian subjects that receive either prophylactic or therapeutic treatment.

An individual is at increased risk of a disease if the subject has at least one known risk-factor (e.g., genetic, biochemical, family history, and situational exposure) placing individuals with that risk factor at a statistically significant greater risk of developing the disease than individuals without the risk factor.

The term “biological sample” refers to a sample of biological material within or obtainable from a biological source, for example a human or mammalian subject. Such samples can be organs, organelles, tissues, sections of tissues, bodily fluids, peripheral blood, blood plasma, blood serum, cells, molecules such as proteins and peptides, and any parts or combinations derived therefrom. The term biological sample can also encompass any material derived by processing the sample. Derived material can include cells or their progeny. Processing of the biological sample may involve one or more of filtration, distillation, extraction, concentration, fixation, inactivation of interfering components, and the like.

The term “control sample” refers to a biological sample not known or suspected to include tau-related disease-affected regions, or at least not known or suspect to include diseased regions of a given type. Control samples can be obtained from individuals not afflicted with the tau-related disease. Alternatively, control samples can be obtained from patients afflicted with the tau-related disease. Such samples can be obtained at the same time as a biological sample thought to comprise the tau-related disease or on a different occasion. A biological sample and a control sample can both be obtained from the same tissue. Preferably, control samples consist essentially or entirely of normal, healthy regions and can be used in comparison to a biological sample thought to comprise tau-related disease-affected regions. Preferably, the tissue in the control sample is the same type as the tissue in the biological sample. Preferably, the tau-related disease-affected cells thought to be in the biological sample arise from the same cell type (e.g., neurons or glia) as the type of cells in the control sample.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease, such as altered gait, as perceived by the subject. A “sign” refers to objective evidence of a disease as observed by a physician.

The term “positive response to treatment” refers to a more favorable response in an individual patient or average response in a population of patients relative to an average response in a control population not receiving treatment.

For purposes of classifying amino acids substitutions as conservative or nonconservative, amino acids are grouped as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same class. Non-conservative substitutions constitute exchanging a member of one of these classes for a member of another.

Percentage sequence identities are determined with antibody sequences maximally aligned by the Kabat numbering convention. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” or “including” one or more recited elements may include other elements not specifically recited. For example, a composition that “comprises” or “includes” an antibody may contain the antibody alone or in combination with other ingredients. When the disclosure refers to a feature comprising specified elements, the disclosure should alternative be understood as referring to the feature consisting essentially of or consisting of the specified elements.

Designation of a range of values includes all integers within or defining the range, and all subranges defined by integers within the range.

Unless otherwise apparent from the context, the term “about” encompasses insubstantial variations, such as values within a standard margin of error of measurement (e.g., SEM) of a stated value.

Statistical significance means p≤0.05.

The singular forms of the articles “a,” “an,” and “the” include plural references unless the context clearly dictates otherwise. For example, the term “a compound” or “at least one compound” can include a plurality of compounds, including mixtures thereof.

### DETAILED DESCRIPTION

### I. General

The invention provides methods of treating taupathies such as Alzheimer's disease with antibodies that bind to human tau.

### II. Target Molecules

Unless otherwise apparent from the context, reference to tau means a natural human form of tau including all isoforms irrespective of whether posttranslational modification (e.g., phosphorylation, glycation, or acetylation) is present. There are six major isoforms (splice variants) of tau occurring in the human brain. The longest of these variants has 441 amino acids, of which the initial met residue is cleaved. Residues are numbered according to the 441 isoform. Thus, for example, reference to a phosphorylation at position 404 means position 404 of the 441 isoform, or corresponding position of any other isoform when maximally aligned with the 441 isoform. The amino acid sequences of the isoforms and Swiss-Prot numbers are indicated below.

```
P10636-8
(SEQ ID NO: 1)
                10 20 30 40 50 60
MAEPRQEFEV MEDHAGTYGL GDRKDQGGYT MHQDQEGDTD AGLKESPLQT PTEDGSEEPG
                70 80 90 100 110 120
SETSDAKSTP TAEDVTAPLV DEGAPGKQAA AQPHTEIPEG TTAEEAGIGD TPSLEDEAAG
              130 140 150 160 170 180
HVTQARMVSK SKDGTGSDDK KAKGADGKTK IATPRGAAPP GQKGQANATR IPAKTPPAPK
              190 200 210 220 230 240
TPPSSGEPPK SGDRSGYSSP GSPGTPGSRS RTPSLPTPPT REPKKVAVVR TPPKSPSSAK
              250 260 270 280 290 300
SRLQTAPVPM PDLKNVKSKI GSTENLKHQP GGGKVQIINK KLDLSNVQSK CGSKDNIKHV
              310 320 330 340 350 360
PGGGSVQIVY KPVDLSKVTS KCGSLGNIHH KPGGGQVEVK SEKLDFKDRV QSKIGSLDNI
              370 380 390 400 410 420
THVPGGGNKK IETHKLTFRE NAKAKTDHGA EIVYKSPVVS GDTSPRHLSN VSSTGSIDMV
              430 440
DSPQLATLAD EVSASLAKQG L
P10636-7
(SEQ ID NO: 2)
                10 20 30 40 50 60
MAEPRQEFEV MEDHAGTYGL GDRKDQGGYT MHQDQEGDTD AGLKESPLQT PTEDGSEEPG
                70 80 90 100 110 120
SETSDAKSTP TAEAEEAGIG DTPSLEDEAA GHVTQARMVS KSKDGTGSDD KKAKGADGKT
              130 140 150 160 170 180
KIATPRGAAP PGQKGQANAT RIPAKTPPAP KTPPSSGEPP KSGDRSGYSS PGSPGTPGSR
              190 200 210 220 230 240
SRTPSLPTPP TREPKKVAVV RTPPKSPSSA KSRLQTAPVP MPDLKNVKSK IGSTENLKHQ
              250 260 270 280 290 300
PGGGKVQIIN KKLDLSNVQS KCGSKDNIKH VPGGGSVQIV YKPVDLSKVT SKCGSLGNIH
              310 320 330 340 350 360
HKPGGGQVEV KSEKLDFKDR VQSKIGSLDN ITHVPGGGNK KIETHKLTFR ENAKAKTDHG
              370 380 390 400 410 420
AEIVYKSPVV SGDTSPRHLS NVSSTGSIDM VDSPQLATLA DEVSASLAKQ GL
P10636-6 (4RON human tau)
(SEQ ID NO: 3)
                10 20 30 40 50 60
MAEPRQEFEV MEDHAGTYGL GDRKDQGGYT MHQDQEGDTD AGLKAEEAGI GDTPSLEDEA
                70 80 90 100 110 120
AGHVTQARMV SKSKDGTGSD DKKAKGADGK TKIATPRGAA PPGQKGQANA TRIPAKTPPA
              130 140 150 160 170 180
PKTPPSSGEP PKSGDRSGYS SPGSPGTPGS RSRTPSLPTP PTREPKKVAV VRTPPKSPSS
              190 200 210 220 230 240
AKSRLQTAPV PMPDLKNVKS KIGSTENLKH QPGGGKVQII NKKLDLSNVQ SKCGSKDNIK
              250 260 270 280 290 300
HVPGGGSVQI VYKPVDLSKV TSKCGSLGNI HHKPGGGQVE VKSEKLDFKD RVQSKIGSLD
              310 320 330 340 350 360
NITHVPGGGN KKIETHKLTF RENAKAKTDH GAEIVYKSPV VSGDTSPRHL SNVSSTGSID
              370 380
MVDSPQLATL ADEVSASLAK QGL
P10636-5
(SEQ ID NO: 4)
                10 20 30 40 50 60
MAEPRQEFEV MEDHAGTYGL GDRKDQGGYT MHQDQEGDTD AGLKESPLQT PTEDGSEEPG
                70 80 90 100 110 120
SETSDAKSTP TAEDVTAPLV DEGAPGKQAA AQPHTEIPEG TTAEEAGIGD TPSLEDEAAG
              130 140 150 160 170 180
HVTQARMVSK SKDGTGSDDK KAKGADGKTK IATPRGAAPP GQKGQANATR IPAKTPPAPK
              190 200 210 220 230 240
TPPSSGEPPK SGDRSGYSSP GSPGTPGSRS RTPSLPTPPT REPKKVAVVR TPPKSPSSAK
              250 260 270 280 290 300
SRLQTAPVPM PDLKNVKSKI GSTENLKHQP GGGKVQIVYK PVDLSKVTSK CGSLGNIHHK
              310 320 330 340 350 360
PGGGQVEVKS EKLDFKDRVQ SKIGSLDNIT HVPGGGNKKI ETHKLTFREN AKAKTDHGAE
              370 380 390 400 410 420
IVYKSPVVSG DTSPRHLSNV SSTGSIDMVD SPQLATLADE VSASLAKQGL
P10636-4
(SEQ ID NO: 5)
                10 20 30 40 50 60
MAEPRQEFEV MEDHAGTYGL GDRKDQGGYT MHQDQEGDTD AGLKESPLQT PTEDGSEEPG
                70 80 90 100 110 120
SETSDAKSTP TAEAEEAGIG DTPSLEDEAA GHVTQARMVS KSKDGTGSDD KKAKGADGKT
              130 140 150 160 170 180
KIATPRGAAP PGQKGQANAT RIPAKTPPAP KTPPSSGEPP KSGDRSGYSS PGSPGTPGSR
              190 200 210 220 230 240
SRTPSLPTPP TREPKKVAVV RTPPKSPSSA KSRLQTAPVP MPDLKNVKSK IGSTENLKHQ
              250 260 270 280 290 300
PGGGKVQIVY KPVDLSKVTS KCGSLGNIHH KPGGGQVEVK SEKLDFKDRV QSKIGSLDNI
              310 320 330 340 350 360
THVPGGGNKK IETHKLTFRE NAKAKTDHGA EIVYKSPVVS GDTSPRHLSN VSSTGSIDMV
              370 380
DSPQLATLAD EVSASLAKQG L
P10636-2
(SEQ ID NO: 6)
                10 20 30 40 50 60
MAEPRQEFEV MEDHAGTYGL GDRKDQGGYT MHQDQEGDTD AGLKAEEAGI GDTPSLEDEA
                70 80 90 100 110 120
AGHVTQARMV SKSKDGTGSD DKKAKGADGK TKIATPRGAA PPGQKGQANA TRIPAKTPPA
              130 140 150 160 170 180
PKTPPSSGEP PKSGDRSGYS SPGSPGTPGS RSRTPSLPTP PTREPKKVAV VRTPPKSPSS
              190 200 210 220 230 240
AKSRLQTAPV PMPDLKNVKS KIGSTENLKH QPGGGKVQIV YKPVDLSKVT SKCGSLGNIH
              250 260 270 280 290 300
HKPGGGQVEV KSEKLDFKDR VQSKIGSLDN ITHVPGGGNK KIETHKLTFR ENAKAKTDHG
              310 320 330 340 350
AEIVYKSPVV SGDTSPRHLS NVSSTGSIDM VDSPQLATLA DEVSASLAKQ GL
```

Reference to tau includes known natural variations about 30 of which are listed in the Swiss-Prot database and permutations thereof, as well as mutations associated with tau pathologies, such as dementia, Pick's disease, supranuclear palsy, etc. (see, e.g., Swiss-Prot database and Poorkaj, et al. Ann Neurol. 43:815-825 (1998)). Some examples of tau mutations numbered by the 441 isoform are a lysine to threonine mutation at amino acid residue 257 (K257T), an isoleucine to valine mutation at amino acid position 260 (1260V); a glycine to valine mutation at amino acid position 272 (G272V); an asparagine to lysine mutation at amino acid position 279 (N279K); an asparagine to histidine mutation at amino acid position 296 (N296H); a proline to serine mutation at amino acid position 301 (P3015); a proline to leucine mutation at amino acid 301 (P301L); a glycine to valine mutation at amino acid position 303 (G303V); a serine to asparagine mutation at position 305 (5305N); a glycine to serine mutation at amino acid position 335 (G335S); a valine to methionine mutation at position 337 (V337M); a glutamic acid to valine mutation at position 342 (E342V); a lysine to isoleucine mutation at amino acid position 369 (K3691); a glycine to arginine mutation at amino acid position 389 (G389R); and an arginine to tryptophan mutation at amino acid position 406 (R406W).

Tau can be phosphorylated at one or more amino acid residues including tyrosine at amino acid positions 18, 29, 97, 310, and 394 serine at amino acid positions 184, 185, 198, 199, 202, 208, 214, 235, 237, 238, 262, 293, 324, 356, 396, 400, 404, 409, 412, 413, and 422; and threonine at amino acids positions 175, 181, 205, 212, 217, 231, and 403. Unless otherwise apparent from context, reference to tau, or their fragments includes the natural human amino acid sequences including isoforms, mutants, and allelic variants thereof.

### III. Antibodies

### A. Binding Specificity and Functional Properties

The invention provides antibodies that bind to tau. Some antibodies specifically bind to an epitope within KXXSXXNX(K/H)H (SEQ ID NO:191). Some antibodies bind to a peptide comprising, consisting essentially of, or consisting of amino acid residues 259-268 of 441 amino acid tau protein (SEQ ID NO:1). Some antibodies bind to a peptide comprising, consisting essentially of, or consisting of amino acid residues 290-299 of 441 amino acid tau protein (SEQ ID NO:1). Some antibodies bind to a peptide comprising, consisting essentially of, or consisting of amino acid residues 321-330 of 441 amino acid tau protein (SEQ ID NO:1). Some antibodies bind to a peptide comprising, consisting essentially of or consisting of amino acid residues 353-362 of 441 amino acid tau protein (SEQ ID NO:1). Some antibodies bind to two, three or all four of these peptides. Some antibodies specifically bind to an epitope within residues 199-213 of 383 amino acid 4RON human tau protein (SEQ ID NO:3) (corresponding to residues 257-271 of SEQ ID NO:1). Some antibodies specifically bind to an epitope within residues 262-276 of 383 amino acid 4RON human tau protein (SEQ ID NO:3) (corresponding to residues 320-334 of SEQ ID NO:1). Some antibodies of the invention specifically bind to a peptide consisting of residues 257-271 of 441 amino acid tau protein (SEQ ID NO:1). Some antibodies of the invention specifically bind to a peptide consisting of residues 320-334 of 441 amino acid tau protein (SEQ ID NO:1). Some antibodies of the invention specifically bind to a peptide consisting of residues 259-268 of 441 amino acid tau protein SEQ ID NO:1, namely KIGSTENLKH (SEQ ID NO:188). Some antibodies of the invention specifically bind to a peptide consisting of residues 290-299 of 441 amino acid tau protein SEQ ID NO:1, namely KCGSKDNIKH (SEQ ID NO:192). Some antibodies of the invention specifically bind to a peptide consisting of residues 321-330 of 441 amino acid tau protein SEQ ID NO:1, namely KCGSLGNIHH (SEQ ID NO:193). Some antibodies of the invention specifically bind to a peptide consisting of residues 353-362 of 441 amino acid tau protein SEQ ID NO:1, namely KIGSLDNITH (SEQ ID NO:194). Some antibodies of the invention specifically bind to a peptide consisting of the consensus motif KXXSXXNX(K/H)H (SEQ ID NO:191). Some antibodies bind to an epitope comprising residues 259, 262, 265, 267, 268, residues 290, 293, 296, 298, 299, residues 321, 324, 327, 329, 330, or residues 353, 356, 359, 362 of 441 amino acid tau protein SEQ ID NO:1. Some antibodies bind to tau irrespective of phosphorylation state. Some antibodies bind to an epitope not including a residue subject to phosphorylation. These antibodies can be obtained by immunizing with a tau polypeptide purified from a natural source or recombinantly expressed. Antibodies can be screened for binding tau in unphosphorylated form as well as a form in which one or more residues susceptible to phosphorylation are phosphorylated. Such antibodies preferably bind with indistinguishable affinities or at least within a factor of 1.5, 2 or 3-fold to phosphorylated tau compared to non-phosphorylated tau (i.e., are “pan-specific”). 3D6 is an example of a pan-specific monoclonal antibody. The invention also provides antibodies binding to the same epitope as any of the foregoing antibodies, such as, for example, the epitope of 3D6. Also included are antibodies competing for binding to tau with any of the foregoing antibodies, such as, for example, competing with 3D6.

The above-mentioned antibodies can be generated de novo by immunizing with a peptide including, consisting essentially of or consisting of residues 199-213 or 262-276 of SEQ ID NO:3 (corresponding to residues 257-271 or 320-334, respectively, of SEQ ID NO:1) or by immunizing with a peptide including, consisting essentially of or consisting of residues 259-268, 290-299, 321-330, or 353-362 of SEQ ID NO:1, or by immunizing with a full length tau polypeptide or fragment thereof comprising such residues and screening for specific binding to a peptide including such residues. Such peptides are preferably attached to a heterologous conjugate molecule that helps elicit an antibody response to the peptide. Attachment can be direct or via a spacer peptide or amino acid. Cysteine is used as a spacer amino acid because its free SH group facilitates attachment of a carrier molecule. A polyglycine linker (e.g., 2-6 glycines), with or without a cysteine residue between the glycines and the peptide can also be used. The carrier molecule serves to provide a T-cell epitope that helps elicit an antibody response against the peptide. Several carriers are commonly used particularly keyhole limpet hemocyanin (KLH), ovalbumin and bovine serum albumin (BSA). Peptide spacers can be added to peptide immunogen as part of solid phase peptide synthesis. Carriers are typically added by chemical cross-linking. Some examples of chemical crosslinkers that can be used include cross-N-maleimido-6-aminocaproyl ester or m-maleimidobenzoyl-N-hydroxysuccinimide ester (MBS) (see for example, Harlow, E. et al., Antibodies: A Laboratory Manual, Cold Spring Harbor Laboratory Press, Cold Spring Harbor, N.Y. 1988; Sinigaglia et al., Nature, 336:778-780 (1988); Chicz et al., J. Exp. Med., 178:27-47 (1993); Hammer et al., Cell 74:197-203 (1993); Falk K. et al., Immunogenetics, 39:230-242 (1994); WO 98/23635; and, Southwood et al. J. Immunology, 160:3363-3373 (1998)). The carrier and spacer if present can be attached to either end of the immunogen.

A peptide with optional spacer and carrier can be used to immunize laboratory animals or B-cells as described in more detail below. Hybridoma supernatants can be tested for ability to bind one or more peptides including, consisting essentially of or consisting of residues 199-213 or 262-276 of SEQ ID NO:3 (corresponding to residues 257-271 or 320-334, respectively, of SEQ ID NO:1), or including, consisting essentially of or consisting of residues 259-268, 290-299, 321-330, or 353-362 of SEQ ID NO:1, and/or phosphorylated and non-phosphorylated forms of tau, such as, for example, a full-length isoform of tau with position 404 in phosphorylated form. The peptide can be attached to a carrier or other tag to facilitate the screening assay. In this case, the carrier or tag is preferentially different than the combination of spacer and carrier molecule used for immunization to eliminate antibodies specific for the spacer or carrier rather than the tau peptide. Any of the tau isoforms can be used.

The invention provides monoclonal antibodies binding to epitopes within tau. An antibody designated 3D6 is one such exemplary mouse antibody. Unless otherwise apparent from context, reference to 3D6 should be understood as referring to any of the mouse, chimeric, veneered, and humanized forms of this antibody. The antibody has been deposited as [DEPOSIT NUMBER]. This antibody specifically binds to an epitope of KXXSXXNX(K/H)H (SEQ ID NO:191). This antibody specifically binds within amino acid residues 199-213 and/or 262-276 of the 383 amino acid 4RON human tau protein (SEQ ID NO:3) (corresponding to amino acid residues 257-271 and/or 320-334, respectively, of SEQ ID NO:1). The antibody specifically binds within amino acid residues 259-268 or 290-299 or 321-330 or 353-362 of SEQ ID NO:1, and combinations of any 2, 3 or all four thereof. This antibody is further characterized by its ability to bind both phosphorylated and unphosphorylated tau, both non-pathological and pathological forms and conformations of tau, and misfolded/aggregated forms of tau. Humanized antibody hu3D6VHv1bA11/L2-DIM4 equivalently binds phosphorylated and non-phosphorylated tau, binds all splice isoforms of tau, and binds neurofibrillary tangles and dystrophic neurites in tissue sections from each of six Alzheimer's disease donor samples tested. An antibody designated 6A10 is one such exemplary mouse antibody. Unless otherwise apparent from context, reference to 6A10 should be understood as referring to any of the mouse, chimeric, veneered, and humanized forms of this antibody. Kabat/Chothia Composite CDRs of the heavy chain of 6A10 are designated SEQ ID NOs:67, 68, and 69, respectively, and Kabat CDRs of the light chain of 6A10 are designated SEQ ID NOs:12, 13, and 14, respectively. Mouse 6A10 shares 82.1% of VH sequence identity and 100% VL sequence identity with the VH chain and VL chain, respectively, of mouse 3D6.

Some antibodies of the invention bind to the same or overlapping epitope as an antibody designated 3D6. The sequences of the heavy and light chain mature variable regions of this antibody are designated SEQ ID NOs:7 and 11, respectively. Other antibodies having such a binding specificity can be produced by immunizing mice with tau or a portion thereof including, consisting essentially of or consisting of the desired epitope (e.g. 199-213 and/or 262-276 of SEQ ID NO:3, corresponding to residues 257-271 and/or 320-334, respectively, of SEQ ID NO:1; or e.g., 259-268 or 290-299 or 321-330 or 353-362 of SEQ ID NO:1, any combination of 2, 3 or all 4 thereof) and screening resulting antibodies for binding to tau optionally in competition with an antibody having the variable regions of mouse 3D6 (IgG1 kappa). Fragments of tau including the desired epitope can be linked to a carrier that helps elicit an antibody response to the fragment and/or be combined with an adjuvant the helps elicit such a response. Such antibodies can be screened for differential binding to tau or a fragment thereof compared with mutants of specified residues. Screening against such mutants more precisely defines the binding specificity to allow identification of antibodies whose binding is inhibited by mutagenesis of particular residues and which are likely to share the functional properties of other exemplified antibodies. The mutations can be systematic replacement substitution with alanine (or serine if an alanine is present already) one residue at a time, or more broadly spaced intervals, throughout the target or throughout a section thereof in which an epitope is known to reside. If the same set of mutations significantly reduces the binding of two antibodies, the two antibodies bind the same epitope.

Antibodies having the binding specificity of a selected murine antibody (e.g., 3D6) can also be produced using a variant of the phage display method. See Winter, WO 92/20791. This method is particularly suitable for producing human antibodies. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions can for example be obtained from a library of rearranged human heavy chain variable regions. A phage showing strong specific binding for tau or a fragment thereof (e.g., at least 10⁸ and preferably at least 10⁹ M⁻¹) is selected. The heavy chain variable region from this phage then serves as a starting material for constructing a further phage library. In this library, each phage displays the same heavy chain variable region (i.e., the region identified from the first display library) and a different light chain variable region. The light chain variable regions can be obtained for example from a library of rearranged human variable light chain regions. Again, phage showing strong specific binding for tau or a fragment thereof are selected. The resulting antibodies usually have the same or similar epitope specificity as the murine starting material.

Kabat/Chothia Composite CDRs of the heavy chain of 3D6 are designated SEQ ID NOs:8, 9, and 10, respectively, and Kabat CDRs of the light chain of 3D6 are designated SEQ ID NOs:12, 13, and 14, respectively.

Table 2 indicates the 3D6 CDRs as defined by Kabat, Chothia, Composite of Chothia and Kabat (also referred to herein as “Kabat/Chothia Composite”), AbM, and Contact.

TABLE 2
3D6 CDRs as defined by Kabat, Chothia, Composite of Chothia and Kabat, AbM, and Contact

| | | | Composite of | | |
|---|---|---|---|---|---|
| | | | Chothia & | | |
| Loop | Kabat | Chothia | Kabat | AbM | Contact |
| L1 | L24--L34 | L24--L34 | L24--L34 | L24--L34 | L30--L36 |
| | SEQ ID NO: 12 | SEQ ID NO: 12 | SEQ ID NO: 12 | SEQ ID NO: 12 | SEQ ID NO: 36 |
| L2 | L50--L56 | L50--L56 | L50--L56 | L50--L56 | L46--L55 |
| | SEQ ID NO: 13 | SEQ ID NO: 13 | SEQ ID NO: 13 | SEQ ID NO: 13 | SEQ ID NO: 37 |
| L3 | L89--L97 | L89--L97 | L89--L97 | L89--L97 | L89--L96 |
| | SEQ ID NO: 14 | SEQ ID NO: 14 | SEQ ID NO: 14 | SEQ ID NO: 14 | SEQ ID NO: 38 |
| H1 | H31--H35B | H26--H32 | H26--H35B | H26--H35B | H30--H35B |
| | SEQ ID NO: 32 | SEQ ID NO: 33 | SEQ ID NO: 8 | SEQ ID NO: 8 | SEQ ID NO: 39 |
| H2 | H50--H65 | H52--H56 | H50--H65 | H50--H58 | H47--H58 |
| | SEQ ID NO: 9 | SEQ ID NO: 34 | SEQ ID NO: 9 | SEQ ID NO: 35 | SEQ ID NO: 40 |
| H3 | H95--H102 | H95--H102 | H95--H102 | H95--H102 | H93--H101 |
| | SEQ ID NO: 10 | SEQ ID NO: 10 | SEQ ID NO: 10 | SEQ ID NO: 10 | SEQ ID NO: 41 |

Other antibodies can be obtained by mutagenesis of cDNA encoding the heavy and light chains of an exemplary antibody, such as 3D6. Monoclonal antibodies that are at least 70%, 80%, 90%, 95%, 96%, 97%, 98%, or 99% identical to 3D6 in amino acid sequence of the mature heavy and/or light chain variable regions and maintain its functional properties, and/or which differ from the respective antibody by a small number of functionally inconsequential amino acid substitutions (e.g., conservative substitutions), deletions, or insertions are also included in the invention. Monoclonal antibodies having at least one or all six CDR(s) as defined by any conventional definition, but preferably Kabat, that are 90%, 95%, 99% or 100% identical to corresponding CDRs of 3D6 are also included.

The invention also provides antibodies having some or all (e.g., 3, 4, 5, and 6) CDRs entirely or substantially from 3D6. Such antibodies can include a heavy chain variable region that has at least two, and usually all three, CDRs entirely or substantially from the heavy chain variable region of 3D6 and/or a light chain variable region having at least two, and usually all three, CDRs entirely or substantially from the light chain variable region of 3D6. The antibodies can include both heavy and light chains. A CDR is substantially from a corresponding 3D6 CDR when it contains no more than 4, 3, 2, or 1 substitutions, insertions, or deletions, except that CDR-H2 (when defined by Kabat) can have no more than 6, 5, 4, 3, 2, or 1 substitutions, insertions, or deletions. Such antibodies can have at least 70%, 80%, 90%, 95%, 96%, 97%, 98%, or 99% identity to 3D6 in the amino acid sequence of the mature heavy and/or light chain variable regions and maintain their functional properties, and/or differ from 3D6 by a small number of functionally inconsequential amino acid substitutions (e.g., conservative substitutions), deletions, or insertions.

Some antibodies identified by such assays can bind to monomeric, misfolded, aggregated, phosphorylated, or unphosphorylated forms of tau or otherwise. Likewise, some antibodies are immunoreactive on non-pathological and pathological forms and conformations of tau.

### B. Non-Human Antibodies

The production of other non-human antibodies, e.g., murine, guinea pig, primate, rabbit or rat, against tau or a fragment thereof (e.g., amino acid residues 199-213 or 262-276 of SEQ ID NO:3, corresponding to amino acid residues 257-271 or 320-334, respectively, of SEQ ID NO:1; or amino acid residues 259-268 or 290-299 or 321-330 or 353-362 of SEQ ID NO:1) can be accomplished by, for example, immunizing the animal with tau or a fragment thereof. See Harlow & Lane, Antibodies, A Laboratory Manual (CSHP NY, 1988) (incorporated by reference for all purposes). Such an immunogen can be obtained from a natural source, by peptide synthesis, or by recombinant expression. Optionally, the immunogen can be administered fused or otherwise complexed with a carrier protein. Optionally, the immunogen can be administered with an adjuvant. Several types of adjuvant can be used as described below. Complete Freund's adjuvant followed by incomplete adjuvant is preferred for immunization of laboratory animals. Rabbits or guinea pigs are typically used for making polyclonal antibodies. Mice are typically used for making monoclonal antibodies. Antibodies are screened for specific binding to tau or an epitope within tau (e.g., an epitope comprising one or more of amino acid residues 199-213 or 262-276 of SEQ ID NO:3; corresponding to amino acid residues 257-271 or 320-334, respectively, of SEQ ID NO:1 or an epitope comprising one or more of amino acid residues 259-268 or 290-299 or 321-330 or 353-362 of SEQ ID NO:1). Such screening can be accomplished by determining binding of an antibody to a collection of tau variants, such as tau variants containing amino acid residues 199-213 or 262-276 of SEQ ID NO:3 (corresponding to amino acid residues 257-271 or 320-334, respectively, of SEQ ID NO:1) or tau variants containing amino acid residues 259-268 or 290-299 or 321-330 or 353-362 of SEQ ID NO:1, or mutations within these residues, and determining which tau variants bind to the antibody. Binding can be assessed, for example, by Western blot, FACS or ELISA.

### C. Humanized Antibodies

A humanized antibody is a genetically engineered antibody in which CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having at least three, four, five or all CDRs entirely or substantially from a donor antibody and variable region framework sequences and constant regions, if present, entirely or substantially from human antibody sequences. Similarly, a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly, a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by any conventional definition but preferably defined by Kabat) are identical between the respective CDRs. The variable region framework sequences of an antibody chain or the constant region of an antibody chain are substantially from a human variable region framework sequence or human constant region respectively when at least 85%, 90%, 95% or 100% of corresponding residues defined by Kabat are identical. To be classified as humanized under the 2014 World Health Organization (WHO) International non-proprietary names (INN) definition of humanized antibodies, an antibody must have at least 85% identity to human germline antibody sequences (i.e., prior to somatic hypermutation). Mixed antibodies are antibodies for which one antibody chain (e.g., heavy chain) meets the threshold but the other chain (e.g., light chain) does not meet the threshold. An antibody is classified as chimeric if neither chain meets the threshold, even though the variable framework regions for both chains were substantially human with some murine backmutations. See, Jones et al. (2016) The INNs and outs of antibody nonproprietary names, mAbs 8:1, 1-9, DOI: 10.1080/19420862.2015.1114320. See also “WHO-INN: International nonproprietary names (INN) for biological and biotechnological substances (a review)” (Internet) 2014. Available from: http://www. who.int/medicines/services/inn/BioRev2014.pdf), incorporated herein by reference. For the avoidance of doubt, the term “humanized” as used herein is not intended to be limited to the 2014 WHO INN definition of humanized antibodies. Some of the humanized antibodies provided herein have at least 85% sequence identity to human germline sequences and some of the humanized antibodies provided herein have less than 85% sequence identity to human germline sequences. Some of the heavy chains of the humanized antibodies provided herein have from about 60% to 100% sequence identity to human germ line sequences, such as, for example, in the range of about 60% to 69%, 70% to 79%, 80% to 84%, or 85% to 89%. Some heavy chains fall below the 2014 WHO INN definition and have, for example, about 64%, 65%, 66%, 67%, 68%, 69%, 70%, 71%, 72%, 73%, 74%, 75%, 76%, 77%, 78%, 79%, 80%, 81%, or 82%, 83%, or 84% sequence identity to human germ line sequences, while other heavy chains meet the 2014 WHO INN definition and have about 85%, 86%, 87%, 88%, 89% or greater sequence identity to human germ line sequences. Some of the light chains of the humanized antibodies provided herein have from about 60% to 100% sequence identity to human germ line sequences, such as, for example, in the range of about 80% to 84% or 85% to 89%. Some light chains fall below the 2014 WHO INN definition and have, for example, about 81%, 82%, 83% or 84% sequence identity to human germ line sequences, while other light chains meet the 2014 WHO INN definition and have about 85%, 86%, 87%, 88%, 89% or greater sequence identity to human germ line sequences. Some humanized antibodies provided herein that are “chimeric” under the 2014 WHO INN definition have heavy chains with less than 85% identity to human germ line sequences paired with light chains having less than 85% identity to human germ line sequences. Some humanized antibodies provided herein are “mixed” under the 2014 WHO INN definition, for example, having a heavy chain with at least 85% sequence identity to human germ line sequences paired with a light chain having less than 85% sequence identity to human germ line sequences, or vice versa. Some humanized antibodies provided herein meet the 2014 WHO INN definition of “humanized” and have a heavy chain with at least 85% sequence identity to human germ line sequences paired with a light chain having at least 85% sequence identity to human germ line sequences. Additional humanized antibodies of the invention meet the 2014 WHO INN definition of “mixed.”

Although humanized antibodies often incorporate all six CDRs (defined by any conventional definition but preferably as defined by Kabat) from a mouse antibody, they can also be made with less than all CDRs (e.g., at least 3, 4, or 5 CDRs) from a mouse antibody (e.g., Pascalis et al., J. Immunol. 169:3076, 2002; Vajdos et al., J. of Mol. Biol., 320: 415-428, 2002; Iwahashi et al., Mol. Immunol. 36:1079-1091, 1999; Tamura et al, J. Immunol., 164:1432-1441, 2000).

In some antibodies only part of the CDRs, namely the subset of CDR residues required for binding, termed the SDRs, are needed to retain binding in a humanized antibody. CDR residues not contacting antigen and not in the SDRs can be identified based on previous studies (for example residues H60-H65 in CDR H2 are often not required), from regions of Kabat CDRs lying outside Chothia hypervariable loops (Chothia, J. Mol. Biol. 196:901, 1987), by molecular modeling and/or empirically, or as described in Gonzales et al., Mol. Immunol. 41: 863, 2004. In such humanized antibodies at positions in which one or more donor CDR residues is absent or in which an entire donor CDR is omitted, the amino acid occupying the position can be an amino acid occupying the corresponding position (by Kabat numbering) in the acceptor antibody sequence. The number of such substitutions of acceptor for donor amino acids in the CDRs to include reflects a balance of competing considerations. Such substitutions are potentially advantageous in decreasing the number of mouse amino acids in a humanized antibody and consequently decreasing potential immunogenicity and/or for meeting the WHO INN definition of “humanized”. However, substitutions can also cause changes of affinity, and significant reductions in affinity are preferably avoided. Positions for substitution within CDRs and amino acids to substitute can also be selected empirically.

The human acceptor antibody sequences can optionally be selected from among the many known human antibody sequences to provide a high degree of sequence identity (e.g., 65-85% identity) between a human acceptor sequence variable region frameworks and corresponding variable region frameworks of a donor antibody chain.

Some humanized and chimeric antibodies have the same (within experimental error) or improved functional properties, e.g., binding affinity for human tau, inhibition of tau internalization into neurons, which can be assayed as described in the examples of US publication 2020/0369755 A1, as a murine antibody from which they were derived. For example, some humanized and chimeric antibodies have a binding affinity within a factor of 3, 2 or 1 of the murine antibody from which they were derived or an affinity indistinguishable within experimental error. Some humanized and chimeric antibodies inhibit tau internalization into neurons, which can be assayed as described in the examples of US publication 2020/0369755 A1, within a factor of 3, 2 or 1 of the murine antibody from which they were derived or inhibit the same within experimental error as the mouse antibody from which they were derived. Some humanized antibodies exhibit reduced immunogenicity, increased affinity, increased thermostability and/or improved expression relative to previously described humanized forms of the 3D6 antibody (see WO 2017/191560 and US publication 2020/0369755 A1) hu3D6VHv1bA11/L2-DIM4 demonstrated improved affinity, as evidenced by on-rate, off-rate, and Kd numbers, over parental hu3D6VHv1bA11/hu3D6VLv2. hu3D6VHv1bA11/L2-DIM4 demonstrated higher thermostability and titer over parental hu3D6VHv1bA11/hu3D6VLv2 (see US publication 2020/0369755 A1). Some antibodies of the present invention bind specific isoforms of tau with high affinity as measured by surface plasmon resonance. For example, hu3D6VHv1bA11/L2-DIM4 binds 3R2N-tau (Swiss-prot IDs: P10636-5) and 4R2N-tau (Swiss-prot IDs: P10636-8) with KDS of 154 pM and 206 pM, respectively.

An example of an acceptor sequence for the heavy chain is the human mature heavy chain variable region of humanized 48G7 Fab with PDB accession code 2RCS-VH_huFrwk (SEQ ID NO:75). The variable domains of 3D6 and 48G7 Fab also share identical lengths for the CDR-H1, H2 loops. An example of an acceptor sequence for the heavy chain is the human mature heavy chain variable region IMGT #IGHV1-69-2*01 (SEQ ID NO:25). IMGT #IGHV1-69-2*01 (SEQ ID NO:25) shares the canonical form of mouse 3D6 heavy chain CDR-H1 and H2. IMGT #IGHV1-69-2*01 (SEQ ID NO:25) belongs to human heavy chain subgroup 1. An example of an acceptor sequence for the light chain is the human mature light chain variable region with PDB accession code human antibody ARX71335 VL (SEQ ID NO:82). The variable light domain of 3D6 and ARX71335 antibody also share identical lengths for the CDR-L1, L2 and L3 loops. An example of an acceptor sequence for the light chain is the human mature light chain variable region with IMGT #IGKV2-30*02 (SEQ ID NO:27). IMGT #IGKV2-30*02 (SEQ ID NO:27) has the same canonical classes for CDR-L1, CDR-L2 and L3 as mouse 3D6. IMGT #IGKV2-30*02 (SEQ ID NO:27) belongs to human kappa subgroup 2.

If more than one human acceptor antibody sequence is selected, a composite or hybrid of those acceptors can be used, and the amino acids used at different positions in the humanized light chain and heavy chain variable regions can be taken from any of the human acceptor antibody sequences used. For example, the human mature heavy chain variable regions of IMGT #IGHV1-69-2*01 (SEQ ID NO:25) and PDB accession code #2RCS-VH_huFrwk (SEQ ID NO:75) were used as acceptor sequences for humanization of the 3D6 mature heavy chain variable region. An example of a positions in which these two acceptors differ is position H17 (T or S). Humanized versions of the 3D6 heavy chain variable region can include either amino acid at this position. For example, the human mature light chain variable regions IMGT #IGKV2-30*02 (SEQ ID NO:27) and PDB code #ARX71335-VL_huFrwk (SEQ ID NO:82) were used as acceptor sequences for humanization of the 3D6 mature light chain variable region. An example of a position in which these two acceptors differ is position L100 (Q or A). Humanized versions of the 3D6 light chain variable region can include either amino acid at this position.

Certain amino acids from the human variable region framework residues can be selected for substitution based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid can be substituted by the equivalent framework amino acid from the mouse antibody when it is reasonably expected that the amino acid:
(1) noncovalently binds antigen directly;
(2) is adjacent to a CDR region or within a CDR as defined by Chothia but not Kabat;
(3) otherwise interacts with a CDR region (e.g., is within about 6 Å of a CDR region), (e.g., identified by modeling the light or heavy chain on the solved structure of a homologous known immunoglobulin chain); or
(4) is a residue participating in the VL-VH interface.

In an embodiment, humanized sequences are generated using a two-stage PCR protocol that allows introduction of multiple mutations, deletions, and insertions using QuikChange site-directed mutagenesis [Wang, W. and Malcolm, B. A. (1999) BioTechniques 26:680-682)].

Framework residues from classes (1) through (3) as defined by Queen, U.S. Pat. No. 5,530,101, are sometimes alternately referred to as canonical and vernier residues. Framework residues that help define the conformation of a CDR loop are sometimes referred to as canonical residues (Chothia & Lesk, J. Mol. Biol. 196:901-917 (1987); Thornton & Martin, J. Mol. Biol. 263:800-815 (1996)). Framework residues that support antigen-binding loop conformations and play a role in fine-tuning the fit of an antibody to antigen are sometimes referred to as vernier residues (Foote & Winter, J. Mol. Biol 224:487-499 (1992)).

Other framework residues that are candidates for substitution are residues creating a potential glycosylation site. Still other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins.

Other framework residues that are candidates for substitution are N-terminal glutamine residues (Q) that may be replaced with glutamic acid (E) to minimize potential for pyroglutamate conversion [Y. Diana Liu, et al., 2011, J. Biol. Chem., 286: 11211-11217]. Glutamic acid (E) conversion to pyroglutamate (pE) occurs more slowly than from glutamine (Q). Because of the loss of a primary amine in the glutamine to pE conversion, antibodies become more acidic. Incomplete conversion produces heterogeneity in the antibody that can be observed as multiple peaks using charge-based analytical methods. Heterogeneity differences may indicate a lack of process control.

Exemplary humanized antibodies are humanized forms of the mouse 3D6, designated Hu3D6.

The mouse antibody 3D6 comprises mature heavy and light chain variable regions having amino acid sequences comprising SEQ ID NO:7 and SEQ ID NO:11, respectively. The invention provides humanized forms of the murine 3D6 antibody including 10 exemplified humanized heavy chain mature variable regions (hu3D6VHvb1 (SEQ ID NO:76), hu3D6VHvb2 (SEQ ID NO:77), hu3D6VHvb3 (SEQ ID NO:78), hu3D6VHvb4 (SEQ ID NO:79), hu3D6VHvb5 (SEQ ID NO:80), hu3D6VHvb6 (SEQ ID NO:90), hu3D6VHvb7 (SEQ ID NO:91), hu3D6VHv1bA11 D60E (h3D6VHvb8, SEQ ID NO:146), hu3D6VHv1bA11 L82cV (SEQ ID NO:147), and hu3D6VHv1bA11 D60E_L80M_Q81E_L82cV_T83R (h3D6VHvb9, SEQ ID NO:148)) and 56 exemplified humanized light chain mature variable regions (hu3D6VLvb1 (SEQ ID NO:83), hu3D6VLvb2 (SEQ ID NO:84), hu3D6VLvb3 (SEQ ID NO:85), hu3D6VLv2 L54D (SEQ ID NO:93), hu3D6VLv2 L54G (SEQ ID NO:94), hu3D6VLv2 L54N (SEQ ID NO:95), hu3D6VLv2 L54E (SEQ ID NO:96), hu3D6VLv2 L50E (SEQ ID NO:97), hu3D6VLv2 L54Q (SEQ ID NO:98), hu3D6VLv2 L50D (SEQ ID NO:99), hu3D6VLv2 L54K (SEQ ID NO:100), hu3D6VLv2 L54R (SEQ ID NO:101), hu3D6VLv2 L54T (SEQ ID NO:102), hu3D6VLv2 L50G (SEQ ID NO:103), hu3D6VLv2 I48G (SEQ ID NO:104), hu3D6VLv2 I48D (SEQ ID NO:105), hu3D6VLv2 L47G (SEQ ID NO:106), hu3D6VLv2 Y49E (SEQ ID NO:107), hu3D6VLv2 L54V (SEQ ID NO:108), hu3D6VLv2 L54S (SEQ ID NO:109), hu3D6VLv2 S52G (SEQ ID NO:110), hu3D6VLv2 L47N (SEQ ID NO:111), hu3D6VLv2 L47D (SEQ ID NO:112), hu3D6VLv2 L47E (SEQ ID NO:113), hu3D6VLv2 L47P (SEQ ID NO:114), hu3D6VLv2 L47T (SEQ ID NO:115), hu3D6VLv2 L47S (SEQ ID NO:116), hu3D6VLv2 L47A (SEQ ID NO:117), hu3D6VLv2 L50V (SEQ ID NO:118), hu3D6VLv2 L37Q_L50G_L54R (SEQ ID NO:119), hu3D6VLv2 L37Q_L50G_L54G (SEQ ID NO:120), hu3D6VLv2 L37Q_S52G_L54G (SEQ ID NO:121), hu3D6VLv2 L37Q_S52G_L54R (SEQ ID NO:122), hu3D6VLv2 L37Q_S52G_L54T (SEQ ID NO:123), hu3D6VLv2 L37Q_S52G_L54D (SEQ ID NO:124), hu3D6VLv2 L37Q_L54R (SEQ ID NO:125), hu3D6VLv2 L37Q_L54G (SEQ ID NO:126), hu3D6VLv2 L37Q_L54D (SEQ ID NO:127), hu3D6VLv2 L37Q_L50G (SEQ ID NO:128), hu3D6VLv2 L37Q_L50D (SEQ ID NO:129), hu3D6VLv2 L37Q_L54T (SEQ ID NO:130), hu3D6VLv2 L37Q_S52G (SEQ ID NO:131), hu3D6VLv2 L37Q_L54E (SEQ ID NO:145), hu3D6VLv2 L37Q_L50D_L54G (SEQ ID NO:132), hu3D6VLv2 L37Q_L50D_L54R (SEQ ID NO:133), hu3D6VLv2 L37Q_L50E_L54G (SEQ ID NO:134), hu3D6VLv2 L37Q_L50E_L54R (SEQ ID NO:135), hu3D6VLv2 L37Q_L50G_L54R_G100Q (SEQ ID NO:136), hu3D6VLv2 L37Q_L50G_L54G_G100Q (SEQ ID NO:137), hu3D6VLv2 L37Q_S52G_L54R_G100Q (SEQ ID NO:138), hu3D6VLv2 L37Q_S52G_L54D_G100Q (SEQ ID NO:139), hu3D6VLv2 L37Q_L50D_L54G_G100Q (SEQ ID NO:140), hu3D6VLv2 L37Q_L50D_L54R_G100Q (SEQ ID NO:141), hu3D6VLv2 L37Q_L50V_L54D_G100Q (SEQ ID NO:142), hu3D6VLv2 L37Q (SEQ ID NO:143), and hu3D6VLv2 G100Q (SEQ ID NO:144)).

FIGS. 2 and 3 of US publication 2020/0369755 A1 show alignments of the heavy chain variable region and light chain variable region, respectively, of murine 3D6 and various humanized antibodies. FIGS. 9A and 9B of US publication 2020/0369755 A1 show alignment of the heavy chain variable region of the murine 3D6 with the heavy chain variable region of various humanized antibodies. FIGS. 10A, 10B, 10C, and 10D of US publication 2020/0369755 A1 show alignment of the light chain variable region of hu3D6VLv2 with the light chain variable region of various humanized antibodies.

For reasons such as possible influence on CDR conformation and/or binding to antigen, mediating interaction between heavy and light chains, interaction with the constant region, being a site for desired or undesired post-translational modification, being an unusual residue for its position in a human variable region sequence and therefore potentially immunogenic, getting aggregation potential, and other reasons, the following 35 variable region framework positions were considered as candidates for substitutions in the 56 exemplified human mature light chain variable regions and the 10 exemplified human mature heavy chain variable regions, as further specified in the examples of US publication 2020/0369755 A1: L7 (T7S), L10 (T10S), L15 (I15L), L17 (Q17E), L37 (L37Q), L45 (K45R), L47 (L47G, L47N, L47D, L47E, L47P, L47T, L47S, or L47A), L48 (I48G or I48D), L49 (Y49E), L83 (L83V), L86 (H86Y), L100 (A100Q), L106 (L106I), H1 (Q1E), H5 (Q5V), H11 (L11V), H17 (S17T), H20 (L20I), H23 (T23K), H38 (K38R), H42 (E42G), H43 (Q43K), H66 (K66R), H67 (A67V), H75 (S75T), H76 (N76D), H80 (L80M), H81 (Q81E), H82c (L82cV), H83 (T83R), H91 (Y91F), H93 (A93S), H94 (S94T), H108 (T108L), and H109 (L109V). The following 9 variable region CDR positions were considered as candidates for substitutions in the 56 exemplified human mature light chain variable regions and 10 exemplified human mature heavy chain variable regions, as further specified in the examples of US publication 2020/0369755 A1: L24 (K24R), L50 (L50E, L50D, L50G, or L50V), L52 (S52G), L54 (L54D, L54G, L54N, L54E, L54Q, L54K, L54R, L54T, L54V, or L54S), H28 (N28T), H54 (N54D), H56 (D56E), H58 (V58I), and H60 (D60E). In some humanized 3D6 antibodies, Kabat CDR-H2 has an amino acid sequence comprising SEQ ID NO:87. In some humanized 3D6 antibodies, Kabat CDR-H2 has an amino acid sequence comprising SEQ ID NO:149. In some humanized 3D6 antibodies, Kabat-Chothia Composite CDR-H1 has an amino acid sequence comprising SEQ ID NO:86, and Kabat CDR-H2 has an amino acid sequence comprising SEQ ID NO:87. In some humanized 3D6 antibodies, Kabat-Chothia Composite CDR-H1 has an amino acid sequence comprising SEQ ID NO:86 and Kabat CDR-H2 has an amino acid sequence comprising SEQ ID NO:88. In some humanized 3D6 antibodies, Kabat-Chothia Composite CDR-H1 has an amino acid sequence comprising SEQ ID NO:86 and Kabat CDR-H2 has an amino acid sequence comprising SEQ ID NO:92. In some humanized 3D6 antibodies, Kabat CDR-L1 has an amino acid sequence comprising SEQ ID NO:89. In some humanized 3D6 antibodies, Kabat CDR-L2 comprises an amino acid sequence selected from the group consisting of SEQ ID NOs:150-175.

Here, as elsewhere, the first-mentioned residue is the residue of a humanized antibody formed by grafting Kabat CDRs or a composite Chothia-Kabat CDR in the case of CDR-H1 into a human acceptor framework, and the second-mentioned residue is a residue being considered for replacing such residue. Thus, within variable region frameworks, the first mentioned residue is human, and within CDRs, the first mentioned residue is mouse.

Exemplified antibodies include any permutations or combinations of the exemplified mature heavy and light chain variable regions VHvb1/VLvb1, VHvb1/VLvb2, VHvb1/VLvb3, VHvb2/VLvb1, VHvb2/VLvb2, VHvb2/VLvb3, VHvb3/VLvb1, VHvb3/VLvb2, VHvb3/VLvb3, VHvb4/VLvb1, VHvb4/VLvb2, VHvb4/VLvb3, VHvb5/VLvb1, VHvb5/VLvb2, VHvb5/VLvb3, VHvb6/VLvb1, VHvb6/VLvb2, VHvb6/VLvb3, VHvb7/VLvb1, VHvb7/VLvb2, VHvb7/VLvb3. Exemplified antibodies include any permutations or combinations of the exemplified mature heavy chain variable regions hu3D6VHvb1 (SEQ ID NO:76), hu3D6VHvb2 (SEQ ID NO:77), hu3D6VHvb3 (SEQ ID NO:78), hu3D6VHvb4 (SEQ ID NO:79), hu3D6Hvb5 (SEQ ID NO:80), hu3D6VHvb6 (SEQ ID NO:90), hu3D6VHvb7 (SEQ ID NO:91), hu3D6VHvb7 (SEQ ID NO:91), hu3D6VHv1bA11 D60E (h3D6VHvb8, SEQ ID NO:146), hu3D6VHv1bA11 L82cV (SEQ ID NO:147), and hu3D6VHv1bA11 D60E_L80M_Q81E_L82cV_T83R (h3D6VHvb9, SEQ ID NO:148) with any of the humanized 3D6VL light chain variable regions hu3D6VLvb1 (SEQ ID NO:83), hu3D6VLvb2 (SEQ ID NO:84), hu3D6VLvb3 (SEQ ID NO:85), hu3D6VLv2 L54D (SEQ ID NO:93), hu3D6VLv2 L54G (SEQ ID NO:94), hu3D6VLv2 L54N (SEQ ID NO:95), hu3D6VLv2 L54E (SEQ ID NO:96), hu3D6VLv2 L50E (SEQ ID NO:97), hu3D6VLv2 L54Q (SEQ ID NO:98), hu3D6VLv2 L50D (SEQ ID NO:99), hu3D6VLv2 L54K (SEQ ID NO:100), hu3D6VLv2 L54R (SEQ ID NO:101), hu3D6VLv2 L54T (SEQ ID NO:102), hu3D6VLv2 L50G (SEQ ID NO:103), hu3D6VLv2 I48G (SEQ ID NO:104), hu3D6VLv2 I48D (SEQ ID NO:105), hu3D6VLv2 L47G (SEQ ID NO:106), hu3D6VLv2 Y49E (SEQ ID NO:107), hu3D6VLv2 L54V (SEQ ID NO:108), hu3D6VLv2 L54S (SEQ ID NO:109), hu3D6VLv2 S52G (SEQ ID NO:110), hu3D6VLv2 L47N (SEQ ID NO:111), hu3D6VLv2 L47D (SEQ ID NO:112), hu3D6VLv2 L47E (SEQ ID NO:113), hu3D6VLv2 L47P (SEQ ID NO:114), hu3D6VLv2 L47T (SEQ ID NO:115), hu3D6VLv2 L47S (SEQ ID NO:116), hu3D6VLv2 L47A (SEQ ID NO:117), hu3D6VLv2 L50V (SEQ ID NO:118), hu3D6VLv2 L37Q_L50G_L54R (SEQ ID NO:119), hu3D6VLv2 L37Q_L50G_L54G (SEQ ID NO:120), hu3D6VLv2 L37Q_S52G_L54G (SEQ ID NO:121), hu3D6VLv2 L37Q_S52G_L54R (SEQ ID NO:122), hu3D6VLv2 L37Q_S52G_L54T (SEQ ID NO:123), hu3D6VLv2 L37Q_S52G_L54D (SEQ ID NO:124), hu3D6VLv2 L37Q_L54R (SEQ ID NO:125), hu3D6VLv2 L37Q_L54G (SEQ ID NO:126), hu3D6VLv2 L37Q_L54D (SEQ ID NO:127), hu3D6VLv2 L37Q_L50G (SEQ ID NO:128), hu3D6VLv2 L37Q_L50D (SEQ ID NO:129), hu3D6VLv2 L37Q_L54T (SEQ ID NO:130), hu3D6VLv2 L37Q_S52G (SEQ ID NO:131), hu3D6VLv2 L37Q_L54E (SEQ ID NO:145), hu3D6VLv2 L37Q_L50D_L54G (SEQ ID NO:132), hu3D6VLv2 L37Q_L50D_L54R (SEQ ID NO:133), hu3D6VLv2 L37Q_L50E_L54G (SEQ ID NO:134), hu3D6VLv2 L37Q_L50E_L54R (SEQ ID NO:135), hu3D6VLv2 L37Q_L50G_L54R_G100Q (SEQ ID NO:136), hu3D6VLv2 L37Q_L50G_L54G_G100Q (SEQ ID NO:137), hu3D6VLv2 L37Q_S52G_L54R_G100Q (SEQ ID NO:138), hu3D6VLv2 L37Q_S52G_L54D_G100Q (SEQ ID NO:139), hu3D6VLv2 L37Q_L50D_L54G_G100Q (SEQ ID NO:140), hu3D6VLv2 L37Q_L50D_L54R_G100Q (SEQ ID NO:141), hu3D6VLv2 L37Q_L50V_L54D_G100Q (SEQ ID NO:142), hu3D6VLv2 L37Q (SEQ ID NO:143), and hu3D6VLv2 G100Q (SEQ ID NO:144).

Exemplified antibodies include any permutations or combinations of the exemplified mature heavy chain variable regions hu3D6VHvb1 (SEQ ID NO:76), hu3D6VHvb2 (SEQ ID NO:77), hu3D6VHvb3 (SEQ ID NO:78), hu3D6VHvb4 (SEQ ID NO:79), hu3D6Hvb5 (SEQ ID NO:80), hu3D6VHvb6 (SEQ ID NO:90), hu3D6VHvb7 (SEQ ID NO:91), hu3D6VHvb7 (SEQ ID NO:91), hu3D6VHv1bA11 D60E (h3D6VHvb8, SEQ ID NO:146), hu3D6VHv1bA11 L82cV (SEQ ID NO:147), and hu3D6VHv1bA11 D60E_L80M_Q81E_L82cV_T83R (h3D6VHvb9, SEQ ID NO: 148) with any of the humanized 3D6VL light chain variable regions hu3D6VLv1 (SEQ ID NO:20), hu3D6VLv2 (SEQ ID NO:21), hu3D6VLv3 (SEQ ID NO:22), and hu3D6VLv4 (SEQ ID NO:22). Exemplified antibodies include any permutations or combinations of the exemplified mature light chain variable regions hu3D6VLvb1 (SEQ ID NO:83), hu3D6VLvb2 (SEQ ID NO:84), hu3D6VLvb3 (SEQ ID NO:85), hu3D6VLv2 L54D (SEQ ID NO:93), hu3D6VLv2 L54G (SEQ ID NO:94), hu3D6VLv2 L54N (SEQ ID NO:95), hu3D6VLv2 L54E (SEQ ID NO:96), hu3D6VLv2 L50E (SEQ ID NO:97), hu3D6VLv2 L54Q (SEQ ID NO:98), hu3D6VLv2 L50D (SEQ ID NO:99), hu3D6VLv2 L54K (SEQ ID NO:100), hu3D6VLv2 L54R (SEQ ID NO:101), hu3D6VLv2 L54T (SEQ ID NO:102), hu3D6VLv2 L50G (SEQ ID NO:103), hu3D6VLv2 I48G (SEQ ID NO:104), hu3D6VLv2 I48D (SEQ ID NO:105), hu3D6VLv2 L47G (SEQ ID NO:106), hu3D6VLv2 Y49E (SEQ ID NO:107), hu3D6VLv2 L54V (SEQ ID NO:108), hu3D6VLv2 L54S (SEQ ID NO:109), hu3D6VLv2 S52G (SEQ ID NO:110), hu3D6VLv2 L47N (SEQ ID NO:111), hu3D6VLv2 L47D (SEQ ID NO:112), hu3D6VLv2 L47E (SEQ ID NO:113), hu3D6VLv2 L47P (SEQ ID NO:114), hu3D6VLv2 L47T (SEQ ID NO:115), hu3D6VLv2 L47S (SEQ ID NO:116), hu3D6VLv2 L47A (SEQ ID NO:117), hu3D6VLv2 L50V (SEQ ID NO:118), hu3D6VLv2 L37Q_L50G_L54R (SEQ ID NO:119), hu3D6VLv2 L37Q_L50G_L54G (SEQ ID NO:120), hu3D6VLv2 L37Q_S52G_L54G (SEQ ID NO:121), hu3D6VLv2 L37Q_S52G_L54R (SEQ ID NO:122), hu3D6VLv2 L37Q_S52G_L54T (SEQ ID NO:123), hu3D6VLv2 L37Q_S52G_L54D (SEQ ID NO:124), hu3D6VLv2 L37Q_L54R (SEQ ID NO:125), hu3D6VLv2 L37Q_L54G (SEQ ID NO:126), hu3D6VLv2 L37Q_L54D (SEQ ID NO:127), hu3D6VLv2 L37Q_L50G (SEQ ID NO:128), hu3D6VLv2 L37Q_L50D (SEQ ID NO:129), hu3D6VLv2 L37Q_L54T (SEQ ID NO:130), hu3D6VLv2 L37Q_S52G (SEQ ID NO:131), hu3D6VLv2 L37Q_L54E (SEQ ID NO:145), hu3D6VLv2 L37Q_L50D_L54G (SEQ ID NO:132), hu3D6VLv2 L37Q_L50D_L54R (SEQ ID NO:133), hu3D6VLv2 L37Q_L50E_L54G (SEQ ID NO:134), hu3D6VLv2 L37Q_L50E_L54R (SEQ ID NO:135), hu3D6VLv2 L37Q_L50G_L54R_G100Q (SEQ ID NO:136), hu3D6VLv2 L37Q_L50G_L54G_G100Q (SEQ ID NO:137), hu3D6VLv2 L37Q_S52G_L54R_G100Q (SEQ ID NO:138), hu3D6VLv2 L37Q_S52G_L54D_G100Q (SEQ ID NO:139), hu3D6VLv2 L37Q_L50D_L54G_G100Q (SEQ ID NO:140), hu3D6VLv2 L37Q_L50D_L54R_G100Q (SEQ ID NO:141), hu3D6VLv2 L37Q_L50V_L54D_G100Q (SEQ ID NO:142), hu3D6VLv2 L37Q (SEQ ID NO:143), and hu3D6VLv2 G100Q (SEQ ID NO:144) with any of the humanized 3D6 heavy chain variable regions hu3D6VHv1 (SEQ ID NO:15); hu3D6VHv2 (SEQ ID NO:16); hu3D6VHv1b (SEQ ID NO:17); hu3D6VHv1bA11 (SEQ ID NO:18); hu3D6VHv5 (SEQ ID NO:19); hu3D6VHv1bA11B6G2 (SEQ ID NO:46); hu3D6VHv1bA11B6H3 (SEQ ID NO:47); hu3D6VHv1c (SEQ ID NO:48); hu3D6VHv1d (SEQ ID NO:49); hu3D6VHv1e (SEQ ID NO:50); hu3D6VHv1f (SEQ ID NO:51); hu3D6VHv3 (SEQ ID NO:52); hu3D6VHv3b (SEQ ID NO:53); hu3D6VHv3c (SEQ ID NO:54); hu3D6VHv4 (SEQ ID NO:55); hu3D6VHv4b (SEQ ID NO:56); and hu3D6VHv4c (SEQ ID NO:57).

The invention provides an antibody in which humanized heavy chain variable region hu3D6VHv1bA11, also known as h3D6Hu5, (SEQ ID NO:18) is combined with humanized light chain variable region hu3D6VLv2 L37Q_S52G_L54R (L2-DIM4, SEQ ID NO:122). The invention provides an antibody in which humanized heavy chain variable region hu3D6VHv1bA11, also known as h3D6Hu5, (SEQ ID NO:18) is combined with humanized light chain variable region hu3D6VLv2 L37Q_S52G_L54T (L2-DIM5, SEQ ID NO:123). The invention provides an antibody in which humanized heavy chain variable region h3D6VHvb8 (SEQ ID NO:146) is combined with humanized light chain variable region hu3D6VLv2 L37Q_S52G_L54R (L2-DIM4, SEQ ID NO:122). The invention provides an antibody in which humanized heavy chain variable region hu3D6VHv1bA11, also known as h3D6Hu5, (SEQ ID NO:18) is combined with humanized light chain variable region hu3D6VLv2 L37Q_S52G_L54G (L2-DIM3, SEQ ID NO:121). The invention provides an antibody in which humanized heavy chain variable region hu3D6VHv1bA11, also known as h3D6Hu5, (SEQ ID NO:18) is combined with humanized light chain variable region hu3D6VLv2 S52G (L2-DIM9, SEQ ID NO:110). The invention provides an antibody in which humanized heavy chain variable region h3D6VHvb8 (SEQ ID NO:146) is combined with humanized light chain variable region hu3D6VLv2 L54G (L2-DIM7, SEQ ID NO:94). The invention provides an antibody in which humanized heavy chain variable region hu3D6VHv1bA11, also known as h3D6Hu5, (SEQ ID NO:18) is combined with humanized light chain variable region hu3D6VLv2 L50G (L2-DIM22, SEQ ID NO: 103).

The invention provides an antibody in which any one of the exemplified humanized heavy chain variable regions is combined with a human heavy chain constant region. An exemplary human heavy chain constant region is provided as SEQ ID NO:176 (IgG1: allotype G1m17,1). For example, SEQ ID NO:178 sets forth the amino acid sequence of a mature heavy chain of a 3D6 humanized variant (hu3D6VHv1bA11 IgG1 G1m17 allotype). For example, SEQ ID NO:180 sets forth the amino acid sequence of a heavy chain of a 3D6 humanized variant (hu3D6VHv1bA11 IgG1 G1m17 allotype) bovine alpha-lactalbumin signal peptide at the N-terminus. The invention provides an antibody in which any one of the exemplified humanized light chain variable regions is combined with a light chain constant region. An exemplary light chain constant region is provided as SEQ ID NO:177 (kappa). For example, SEQ ID NO:179 sets forth the amino acid sequence of a mature light chain of a 3D6 humanized variant (hu3D6VLv2 variant L37Q_S52G_L54R, L2-DIM4 kappa). For example, SEQ ID NO:181 sets forth the amino acid sequence of a light chain of a 3D6 humanized variant (hu3D6VLv2 variant L37Q_S52G_L54R, L2-DIM4 kappa) with bovine alpha-lactalbumin signal peptide at the N-terminus.

The invention provides variants of the 3D6 humanized antibody in which the humanized mature heavy chain variable region shows at least 90%, 95%, 96%, 97%, 98%, or 99% identity to hu3D6VHvb1 (SEQ ID NO:76), hu3D6VHvb2 (SEQ ID NO:77), hu3D6VHvb3 (SEQ ID NO:78), hu3D6VHvb4 (SEQ ID NO:79), hu3D6Hvb5 (SEQ ID NO:80), hu3D6VHvb6 (SEQ ID NO:90), hu3D6VHvb7 (SEQ ID NO:91), hu3D6VHvb7 (SEQ ID NO:91), hu3D6VHv1bA11 D60E (h3D6VHvb8, SEQ ID NO:146), hu3D6VHv1bA11 L82cV (SEQ ID NO:147), or hu3D6VHv1bA11 D60E_L80M_Q81E_L82cV_T83R (h3D6VHvb9, SEQ ID NO:148) and the humanized mature light chain variable region shows at least 90%, 95%, 96%, 97%, 98%, or 99% identity to hu3D6VLvb1 (SEQ ID NO:83), hu3D6VLvb2 (SEQ ID NO:84), hu3D6VLvb3 (SEQ ID NO:85), hu3D6VLv2 L54D (SEQ ID NO:93), hu3D6VLv2 L54G (SEQ ID NO:94), hu3D6VLv2 L54N (SEQ ID NO:95), hu3D6VLv2 L54E (SEQ ID NO:96), hu3D6VLv2 L50E (SEQ ID NO:97), hu3D6VLv2 L54Q (SEQ ID NO:98), hu3D6VLv2 L50D (SEQ ID NO:99), hu3D6VLv2 L54K (SEQ ID NO:100), hu3D6VLv2 L54R (SEQ ID NO:101), hu3D6VLv2 L54T (SEQ ID NO:102), hu3D6VLv2 L50G (SEQ ID NO:103), hu3D6VLv2 I48G (SEQ ID NO:104), hu3D6VLv2 I48D (SEQ ID NO:105), hu3D6VLv2 L47G (SEQ ID NO:106), hu3D6VLv2 Y49E (SEQ ID NO:107), hu3D6VLv2 L54V (SEQ ID NO:108), hu3D6VLv2 L54S (SEQ ID NO:109), hu3D6VLv2 S52G (SEQ ID NO:110), hu3D6VLv2 L47N (SEQ ID NO:111), hu3D6VLv2 L47D (SEQ ID NO:112), hu3D6VLv2 L47E (SEQ ID NO:113), hu3D6VLv2 L47P (SEQ ID NO:114), hu3D6VLv2 L47T (SEQ ID NO:115), hu3D6VLv2 L47S (SEQ ID NO:116), hu3D6VLv2 L47A (SEQ ID NO:117), hu3D6VLv2 L50V (SEQ ID NO:118), hu3D6VLv2 L37Q_L50G_L54R (SEQ ID NO: 119), hu3D6VLv2 L37Q_L50G_L54G (SEQ ID NO:120), hu3D6VLv2 L37Q_S52G_L54G (SEQ ID NO:121), hu3D6VLv2 L37Q_S52G_L54R (SEQ ID NO:122), hu3D6VLv2 L37Q_S52G_L54T (SEQ ID NO:123), hu3D6VLv2 L37Q_S52G_L54D (SEQ ID NO:124), hu3D6VLv2 L37Q_L54R (SEQ ID NO:125), hu3D6VLv2 L37Q_L54G (SEQ ID NO:126), hu3D6VLv2 L37Q_L54D (SEQ ID NO:127), hu3D6VLv2 L37Q_L50G (SEQ ID NO:128), hu3D6VLv2 L37Q_L50D (SEQ ID NO:129), hu3D6VLv2 L37Q_L54T (SEQ ID NO:130), hu3D6VLv2 L37Q_S52G (SEQ ID NO:131), hu3D6VLv2 L37Q_L54E (SEQ ID NO:145), hu3D6VLv2 L37Q_L50D_L54G (SEQ ID NO:132), hu3D6VLv2 L37Q_L50D_L54R (SEQ ID NO:133), hu3D6VLv2 L37Q_L50E_L54G (SEQ ID NO:134), hu3D6VLv2 L37Q_L50E_L54R (SEQ ID NO:135), hu3D6VLv2 L37Q_L50G_L54R_G100Q (SEQ ID NO:136), hu3D6VLv2 L37Q_L50G_L54G_G100Q (SEQ ID NO:137), hu3D6VLv2 L37Q_S52G_L54R_G100Q (SEQ ID NO:138), hu3D6VLv2 L37Q_S52G_L54D_G100Q (SEQ ID NO:139), hu3D6VLv2 L37Q_L50D_L54G_G100Q (SEQ ID NO:140), hu3D6VLv2 L37Q_L50D_L54R_G100Q (SEQ ID NO:141), hu3D6VLv2 L37Q_L50V_L54D_G100Q (SEQ ID NO:142), hu3D6VLv2 L37Q (SEQ ID NO:143), or hu3D6VLv2 G100Q (SEQ ID NO:144). In some such antibodies at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or all 44 of the backmutations or other mutations in SEQ ID NOs:76-80, SEQ ID NOs:90-91, SEQ ID NOs:146-148, SEQ ID NOs:83-85, and SEQ ID NOs:93-145 are retained.

In some humanized 3D6 antibodies, at least one of the following positions in the VH region is occupied by the amino acid as specified: H93 is occupied by S and H94 is occupied by T. In some humanized 3D6 antibodies, positions H93 and H94 are occupied by S and T, respectively.

In some humanized 3D6 antibodies, position H91 in the VH region is occupied by F.

In some humanized 3D6 antibodies, at least one of the following positions in the VH region is occupied by the amino acid as specified: H1 is occupied by E, H5 is occupied by V, H11 is occupied by V, H20 is occupied I, H23 is occupied by K, H38 is occupied by R, H42 is occupied by G, H43 is occupied by K, H66 is occupied by R, H75 is occupied by T, H76 is occupied by D, H81 is occupied by E, H108 is occupied by L, H109 is occupied by V. In some humanized 3D6 antibodies, positions H1, H5, H11, H20, H23, H38, H42, H43, H66, H75, H76, H81, H108, and H109 in the VH region are occupied by E, V, V, I, K, R, G, K, R, T, D, E, L, and V, respectively.

In some humanized 3D6 antibodies, at least one of the following positions in the VH region is occupied by the amino acid as specified: H17 is occupied by T, H80 is occupied by M, H83 is occupied by R. In some humanized 3D6 antibodies, positions H17, H80, and H83 in the VH region are occupied by T, M, and R, respectively.

In some humanized 3D6 antibodies, position H58 in the VH region is occupied by I.

In some humanized 3D6 antibodies, at least one of the following positions in the VH region is occupied by the amino acid as specified: H28 is occupied by T, H67 is occupied by V. In some humanized 3D6 antibodies, positions H28 and H67 in the VH region are occupied by T and V, respectively.

In some humanized 3D6 antibodies, at least one of the following positions in the VH region is occupied by the amino acid as specified: H54 is occupied by D, H56 is occupied by E. In some humanized 3D6 antibodies, positions H54 and H56 in the VH region are occupied by D and E, respectively.

In some humanized 3D6 antibodies, at least one of the following positions in the VH region is occupied by the amino acid as specified: H1 is occupied by Q or E, H5 is occupied by Q or V, H11 is occupied by L or V, H17 is occupied by S or T, H20 is occupied by L or I, H23 is occupied by T or K, H28 is occupied by N or T, H38 is occupied by K or R, H42 is occupied by E or G, H43 is occupied by Q or K, H54 is occupied by N or D, H56 is occupied by D or E, H58 is occupied by V or I, H66 is occupied by K or R, H67 is occupied by A or V, H75 is occupied by S or T, H76 is occupied by N or D, H80 is occupied by L or M, H81 is occupied by Q or E, H83 is occupied by T or R, H91 is occupied by F or Y, H93 is occupied by S, H94 is occupied by T, H108 is occupied by T or L, H109 is occupied by L or V.

In some humanized 3D6 antibodies, positions H91, H93, and H94 in the VH region are occupied by F, S, and T, respectively, as in huVHvb1. In some humanized 3D6 antibodies, positions H1, H5, H11, H20, H23, H38, H42, H43, H66, H75, H76, H81, H91, H93, H94, H108, and H109 in the VH region are occupied by E, V, V, I, K, R, G, K, R, T, D, E, F, S, T, L, and V, respectively, as in huVHvb2. In some humanized 3D6 antibodies, positions H1, H5, H11, H17, H20, H23, H38, H42, H43, H58, H66, H75, H76, H80, H81, H83, H93, H94, H108, and H109 in the VH region are occupied by E, V, V, T, I, K, R, G, K, I, R, T, D, M, E, R, S, T, L, and V, respectively, as in huVHvb3. In some humanized 3D6 antibodies, positions H1, H5, H11, H17, H20, H23, H28, H38, H42, H43, H58, H66, H67, H75, H76, H80, H81, H83, H93, H94, H108, and H109 in the VH region are occupied by E, V, V, T, I, K, T, R, G, K, I, R, V, T, D, M, E, R, S, T, L, and V, respectively, as in huVHvb4. In some humanized 3D6 antibodies, positions H1, H5, H11, H17, H20, H23, H28, H38, H42, H43, H54, H56, H58, H66, H67, H75, H76, H80, H81, H83, H93, H94, H108, and H109 in the VH region are occupied by E, V, V, T, I, K, T, R, G, K, D, E, I, R, V, T, D, M, E, R, S, T, L, and V, respectively, as in huVHvb5. In some humanized 3D6 antibodies, positions H1, H5, H11, H17, H20, H23, H28, H38, H42, H43, H54, H56, H66, H67, H75, H76, H80, H81, H83, H91, H93, H94, H108, and H109 in the VH region are occupied by E, V, V, T, I, K, T, R, G, K, D, E, R, V, T, D, M, E, R, F, S, T, L, and V, respectively, as in huVHvb6. In some humanized 3D6 antibodies, positions H1, H5, H11, H17, H20, H23, H28, H38, H42, H43, H54, H56, H66, H67, H75, H76, H80, H81, H83, H93, H94, H108, and H109 in the VH region are occupied by E, V, V, T, I, K, T, R, G, K, D, E, R, V, T, D, M, E, R, S, T, L, and V, respectively, as in huVHvb7.

In some humanized 3D6 antibodies, position H60 is occupied by E, as in hu3D6VHv1bA11 D60E (h3D6VHvb8). In some humanized 3D6 antibodies, position H82C is occupied by V, as in hu3D6VHv1bA11 L82cV. In some humanized 3D6 antibodies, positions H60, H80, H81, H82c, and H83 are occupied by E, M, E, V, and R, as in hu3D6VHv1bA11 D60E_L80M_Q81E_L82cV_T83R (h3D6VHvb9).

The heavy chain variable region of any of the above-referenced antibodies can be modified to further reduce immunogenicity. For example, in some of the humanized antibodies position H80 is occupied by M and/or position H82c is occupied by V.

In some humanized 3D6 antibodies, at least one of the following positions in the VL region is occupied by the amino acid as specified: L7 is occupied by S, L10 is occupied by S, L15 is occupied by L, L83 is occupied by V, L86 is occupied by Y, and L106 is occupied by I. In some humanized 3D6 antibodies, positions L7, L10, L15, L83, L86, and L106 are occupied by S, S, L, V, Y, and Y, respectively.

In some humanized 3D6 antibodies, at least one of the following positions in the VL region is occupied by the amino acid as specified: L7 is T or S, L10 is T or S, L15 is I or L, L17 is Q or E, L24 is K or R, L37 is L or Q, L45 is K or R, L83 is L or V, L86 is H or Y, L100 is A or Q, L106 is L or I.

In some humanized 3D6 antibodies, positions L7, L10, L15, L83, L86, and L106 in the VL region are occupied by S, S, L, V, Y, and I, respectively, as in huVLvb2. In some humanized 3D6 antibodies, positions L7, L10, L15, L17, L24, L37, L45, L83, L86, L100, and L106 in the VL region are occupied by S, S, L, E, R, Q, R, V, Y, Q, and I, respectively, as in huVLvb3.

The light chain variable region of any of the above referenced antibodies can be modified to further reduce immunogenicity. For example, in some of the humanized antibodies position L47 is occupied by G, N, D, E, P, T, S or A; position L48 is occupied by G or D; position L49 is occupied by E; position L50 is occupied by E, D, G, or V; position L52 is occupied by G; and/or position L54 is occupied by D, G, N, E, Q, K, R, T, V or S. The heavy chain variable region of any of the above-referenced antibodies can be modified to further reduce immunogenicity. For example, in some of the humanized antibodies position H80 is occupied by M and/or position H82c is occupied by V.

In some humanized 3D6 antibodies, position L54 is occupied by D, as in hu3D6VLv2 L54D. In some humanized 3D6 antibodies, position L54 is occupied by G, as in hu3D6VLv2 L54G. In some humanized 3D6 antibodies, position L54 is occupied by N, as in hu3D6VLv2 L54N, In some humanized 3D6 antibodies, position L54 is occupied by E, as in hu3D6VLv2 L54E. In some humanized 3D6 antibodies, position L50 is occupied by E, as in hu3D6VLv2 L50E. In some humanized 3D6 antibodies, position L54 is occupied by Q, as in hu3D6VLv2 L54Q. In some humanized 3D6 antibodies, position L50 is occupied by D, as in hu3D6VLv2 L50D. In some humanized 3D6 antibodies, position L54 is occupied by K, as in hu3D6VLv2 L54K. In some humanized 3D6 antibodies, position L54 is occupied by R, as in hu3D6VLv2 L54R. In some humanized 3D6 antibodies, position L54 is occupied by T, as in hu3D6VLv2 L54T. In some humanized 3D6 antibodies, position L50 is occupied by G, as in hu3D6VLv2 L50G. In some humanized 3D6 antibodies, position L48 is occupied by G, as in hu3D6VLv2 I48G. In some humanized 3D6 antibodies, position L48 is occupied by D, as in hu3D6VLv2 I48D. In some humanized 3D6 antibodies, position L47 is occupied by G, as in hu3D6VLv2 L47G. In some humanized 3D6 antibodies, position L49 is occupied by E, as in hu3D6VLv2 Y49E. In some humanized 3D6 antibodies, position L54 is occupied by V, as in hu3D6VLv2 L54V. In some humanized 3D6 antibodies, position L54 is occupied by S, as in hu3D6VLv2 L54S. In some humanized 3D6 antibodies, position L52 is occupied by G, as in hu3D6VLv2 S52G. In some humanized 3D6 antibodies, position L47 is occupied by N, as in hu3D6VLv2 L47N. In some humanized 3D6 antibodies, position L47 is occupied by D, as in hu3D6VLv2 L47D. In some humanized 3D6 antibodies, position L47 is occupied by E, as in hu3D6VLv2 L47E. In some humanized 3D6 antibodies, position L47 is occupied by P, as in hu3D6VLv2 L47P. In some humanized 3D6 antibodies, position L47 is occupied by T, as in hu3D6VLv2 L47T. In some humanized 3D6 antibodies, position L47 is occupied by S, as in hu3D6VLv2 L47S. In some humanized 3D6 antibodies, position L47 is occupied by A, as in hu3D6VLv2 L47A. In some humanized 3D6 antibodies, position L50 is occupied by V, as in hu3D6VLv2 L50V.

In some humanized 3D6 antibodies, positions L37, L50, and L54 are occupied by Q, G, and R, respectively, as in hu3D6VLv2 L37Q_L50G_L54R. In some humanized 3D6 antibodies, positions L37, L50, and L54 are occupied by Q, G, and G, respectively, as in hu3D6VLv2 L37Q_L50G_L54G. In some humanized 3D6 antibodies, positions L37, L52, and L54 are occupied by Q, G, and G, respectively, as in hu3D6VLv2 L37Q_S52G_L54G. In some humanized 3D6 antibodies, positions L37, L52, and L54 are occupied by Q, G, and R, respectively, as in hu3D6VLv2 L37Q_S52G_L54R. In some humanized 3D6 antibodies, positions L37, L52, and L54 are occupied by Q, G, and T, respectively, as in hu3D6VLv2 L37Q_S52G_L54T. In some humanized 3D6 antibodies, positions L37, L52, and L54 are occupied by Q, G, and D, respectively, as in hu3D6VLv2 L37Q_S52G_L54D.

In some humanized 3D6 antibodies, positions L37 and L54 are occupied Q and R, respectively, as in hu3D6VLv2 L37Q_L54R. In some humanized 3D6 antibodies, positions L37 and L54 are occupied by Q and G, respectively, as in hu3D6VLv2 L37Q_L54G. In some humanized 3D6 antibodies, positions L37 and L54 are occupied by Q and D, respectively, as in hu3D6VLv2 L37Q_L54D. In some humanized 3D6 antibodies, positions L37 and L50 are occupied by Q and G, respectively, as in hu3D6VLv2 L37Q_L50G. In some humanized 3D6 antibodies, positions L37 and L50 are occupied by Q and D, respectively, as in hu3D6VLv2 L37Q_L50D. In some humanized 3D6 antibodies, positions L37 and L54 are occupied by Q and T, respectively, as in hu3D6VLv2 L37Q_L54T. In some humanized 3D6 antibodies, positions L37 and L52 are occupied by Q and G, respectively, as in hu3D6VLv2 L37Q_S52G. In some humanized 3D6 antibodies, positions L37 and L54 are occupied by Q and E, respectively, as in hu3D6VLv2 L37Q_L54E.

In some humanized 3D6 antibodies, positions L37, L50, and L54 are occupied by Q, D, and G, respectively, as in hu3D6VLv2 L37Q_L50D_L54G. In some humanized 3D6 antibodies, positions L37, L50, and L54 are occupied by Q, D, and R, respectively, as in hu3D6VLv2 L37Q_L50D_L54R. In some humanized 3D6 antibodies, positions L37, L50, and L54 are occupied by Q, E, and G, respectively, as in hu3D6VLv2 L37Q_L50E_L54G. In some humanized 3D6 antibodies, positions L37, L50, and L54 are occupied by Q, E, and R, respectively, as in hu3D6VLv2 L37Q_L50E_L54R.

In some humanized 3D6 antibodies, positions L37, L50, L54, and L100 are occupied by Q, G, R, and Q, respectively, as in hu3D6VLv2 L37Q_L50G_L54R_G100Q. In some humanized 3D6 antibodies, positions L37, L50, L54, and L100 are occupied by Q, G, G, and Q, respectively, as in hu3D6VLv2 L37Q_L50G_L54G_G100Q. In some humanized 3D6 antibodies, positions L37, L52, L54, and L100 are occupied by Q, G, R, and Q, respectively, as in hu3D6VLv2 L37Q_S52G_L54R_G100Q. In some humanized 3D6 antibodies, positions L37, L52, L54, and L100 are occupied by Q, G, D, and Q, respectively, as in hu3D6VLv2 L37Q_S52G_L54D_G100Q. In some humanized 3D6 antibodies, positions L37, L50, L54, and L100 are occupied by Q, D, G, and Q, respectively, as in hu3D6VLv2 L37Q_L50D_L54G_G100Q. In some humanized 3D6 antibodies, positions L37, L50, L54, and L100 are occupied by Q, D, R, and Q, respectively, as in hu3D6VLv2 L37Q_L50D_L54R_G100Q. In some humanized 3D6 antibodies, positions L37, L50, L54, and L100 are occupied by Q, V, D, and Q, respectively, as in hu3D6VLv2 L37Q_L50V_L54D_G100Q.

In some humanized 3D6 antibodies, position L37 is occupied by Q, as in hu3D6VLv2 L37Q. In some humanized 3D6 antibodies, position L100 is occupied by Q as in hu3D6VLv2 G100Q.

Some humanized 3D6 antibodies comprise a mature heavy chain variable region comprising CDRs H1, H2 and H3 comprising SEQ ID NOs:8, 9, and 10, respectively except that position H28 can be occupied by N or T, H54 can be occupied by N or D, H56 can be occupied by D or E, position H58 occupied by V or I, and position H60 can be occupied by D or E, and a mature light chain variable region comprising CDRs L1, L2 and L3 comprising SEQ ID NOs.: 12, 13, and 14 respectively, except that position L24 can be occupied by K or R, position L50 can be occupied by L, E, D, G, or V, position L52 can be occupied by S or G, and position L54 can be occupied by L, D, G, N, E, Q, K, R, T, V, or S, wherein at least one of the following positions is occupied by the amino acid as specified: H1 is occupied by Q, H5 is occupied by Q, H11 is occupied by L, H20 is occupied by L, H23 is occupied by T, H38 is occupied by K, H75 is occupied by S, H56 is occupied by E, H58 is occupied by I, H60 is occupied by E, H82c is occupied by V, L10 is occupied by T, L17 is occupied by E, L24 is occupied by R, L37 is occupied by Q, L47 is occupied by G, N, D, E, P, T, S, or A, L48 is occupied by G or D, L49 is occupied by E, L50 is occupied by E, D, G, or V, L52 is occupied by G, L54 is occupied by D, G, N, E, Q, K, R, T, V, or S, L83 is occupied by L, L86 is occupied by H, L100 is occupied by Q, L106 is occupied by L.

Some humanized 3D6 antibodies comprise three light chain CDRs and three heavy chain CDRs of monoclonal antibody 3D6, wherein 3D6 is a mouse antibody characterized by a heavy chain variable region having an amino acid sequence comprising SEQ ID NO:7 and a light chain variable region having an amino acid sequence comprising SEQ ID NO:11, except that position H27 can be occupied by F or Y, position H28 can be occupied by N or T, position H29 can be occupied by I or F, position H30 can be occupied by K or T, position H51 can be occupied by I or V, position H54 can be occupied by N or D, position H60 can be occupied by D, A, or E, position H61 can be occupied by P or E, position H102 can be occupied by F or Y, position L50 can be occupied by L, E, D, G, or V, position L52 can be occupied by S or G, and position L54 can be occupied by L, D, G, N, E, Q, K, R, T, V, or S, wherein at least one of the following positions is occupied by the amino acid as specified: L37 is occupied by Q, L47 is occupied by G, N, D, E, P, T, S, or A, L48 is occupied by G or D, L49 is occupied by E, L50 is occupied by E, D, G, or V, L52 is occupied by G, L54 is occupied by D, G, N, E, Q, K, R, T, V, or S, L100 is occupied by Q, H60 is occupied by E, H82c is occupied by V.

In some humanized 3D6 antibodies, the variable heavy chain has ≥85% identity to human sequence. In some humanized 3D6 antibodies, the variable light chain has ≥85% identity to human sequence. In some humanized 3D6 antibodies, each of the variable heavy chain and variable light chain has ≥85% identity to human germline sequence. In some humanized 3D6 antibodies, the three heavy chain CDRs are as defined by Kabat/Chothia Composite (SEQ ID NOs:8, 9, and 10) and the three light chain CDRs are as defined by Kabat/Chothia Composite (SEQ ID NOs:12, 13, and 14); provided that position H28 is occupied by N or T, position H54 is occupied by N or D, position H56 is occupied by D or E, position H58 is occupied by V or I, position H60 is occupied by D or E, position L24 is occupied by K or R, position L50 is occupied by L, E, D, G, or V, position L52 is occupied by S or G, and position L54 is occupied by L, D, G, N, E, Q, K, R, T, V, or S. In some humanized 3D6 antibodies, Kabat/Chothia Composite CDR-H1 has an amino acid sequence comprising SEQ ID NO:86. In some humanized 3D6 antibodies, Kabat CDR-H2 has an amino acid sequence comprising SEQ ID NO:87, SEQ ID NO:88, SEQ ID NO:92, or SEQ ID NO:149. In some humanized 3D6 antibodies, Kabat CDR-L1 has an amino acid sequence comprising SEQ ID NO:89. In some humanized 3D6 antibodies, Kabat CDR-L2 comprises an amino acid sequence selected from the group consisting of SEQ ID NOs:150-175.

The CDR regions of such humanized antibodies can be identical or substantially identical to the CDR regions of 3D6, The CDR regions can be defined by any conventional definition (e.g., Chothia, or composite of Chothia and Kabat) but are preferably as defined by Kabat.

Variable regions framework positions are in accordance with Kabat numbering unless otherwise stated. Other such variants typically differ from the sequences of the exemplified Hu3D6 heavy and light chains by a small number (e.g., typically no more than 1, 2, 3, 5, 10, or 15) of replacements, deletions or insertions. Such differences are usually in the framework but can also occur in the CDRs.

A possibility for additional variation in humanized 3D6 variants is additional backmutations in the variable region frameworks. Many of the framework residues not in contact with the CDRs in the humanized mAb can accommodate substitutions of amino acids from the corresponding positions of the donor mouse mAb or other mouse or human antibodies, and even many potential CDR-contact residues are also amenable to substitution. Even amino acids within the CDRs may be altered, for example, with residues found at the corresponding position of the human acceptor sequence used to supply variable region frameworks. In addition, alternate human acceptor sequences can be used, for example, for the heavy and/or light chain. If different acceptor sequences are used, one or more of the backmutations recommended above may not be performed because the corresponding donor and acceptor residues are already the same without backmutations.

Preferably, replacements or backmutations in humanized 3D6 variants (whether or not conservative) have no substantial effect on the binding affinity or potency of the humanized mAb, that is, its ability to bind to tau.

The humanized 3D6 antibodies are further characterized by their ability to bind both phosphorylated and unphosphorylated tau and misfolded/aggregated forms of tau.

### D. Chimeric and Veneered Antibodies

The invention further provides chimeric and veneered forms of non-human antibodies, particularly the 3D6 antibodies of the examples.

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence. In an embodiment, a chimeric 3D6 antibody has a heavy chain amino acid sequence of SEQ ID NO:72 and a light chain amino acid sequence of SEQ ID NO:73.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions. Veneered forms of the 3D6 antibody are included in the invention.

### E. Human Antibodies

Human antibodies against tau or a fragment thereof (e.g., amino acid residues 199-213 and/or 262-276 of SEQ ID NO:3, corresponding to amino acid residues 257-271 and/or 320-334, respectively, of SEQ ID NO:1 or amino acid residues 259-268 or 290-299 or 321-330 or 353-362 of SEQ ID NO:1 or any combination of 2, 3 or all 4 thereof) are provided by a variety of techniques described below. Some human antibodies are selected by competitive binding experiments, by the phage display method of Winter, above, or otherwise, to have the same epitope specificity as a particular mouse antibody, such as one of the mouse monoclonal antibodies described in the examples. Human antibodies can also be screened for a particular epitope specificity by using only a fragment of tau, such as a tau fragment containing only amino acid residues 199-213 or 262-276 of SEQ ID NO:3 (corresponding to amino acid residues 257-271 or 320-334, respectively, of SEQ ID NO:1) or containing only amino acid residues 259-268 or 290-299 or 321-330 or 353-362 of SEQ ID NO:1, as the target antigen, and/or by screening antibodies against a collection of tau variants, such as tau variants containing various mutations within amino acid residues 199-213 or 262-276 of SEQ ID NO:3 (corresponding to amino acid residues 257-271 or 320-334, respectively, of SEQ ID NO:1), or within amino acid residues 259-268 or 290-299 or 321-330 or 353-362 of SEQ ID NO:1.

Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. Nos. 5,877,397; 5,874,299; 5,814,318; 5,789,650; 5,770,429; 5,661,016; 5,633,425; 5,625,126; 5,569,825; 5,545,806; Neuberger, Nat. Biotechnol. 14:826 (1996); and Kucherlapati, WO 91/10741 (1991)) phage display methods (see, e.g., Dower et al., WO 91/17271; McCafferty et al., WO 92/01047; U.S. Pat. Nos. 5,877,218; 5,871,907; 5,858,657; 5,837,242; 5,733,743; and 5,565,332); and methods described in WO 2008/081008 (e.g., immortalizing memory B cells isolated from humans, e.g., with EBV, screening for desired properties, and cloning and expressing recombinant forms).

### F. Selection of Constant Region

The heavy and light chain variable regions of chimeric, veneered or humanized antibodies can be linked to at least a portion of a human constant region. The choice of constant region depends, in part, whether antibody-dependent cell-mediated cytotoxicity, antibody dependent cellular phagocytosis and/or complement dependent cytotoxicity are desired. For example, human isotypes IgG1 and IgG3 have complement-dependent cytotoxicity and human isotypes IgG2 and IgG4 do not. Human IgG1 and IgG3 also induce stronger cell mediated effector functions than human IgG2 and IgG4. Light chain constant regions can be lambda or kappa. Numbering conventions for constant regions include EU numbering (Edelman, G. M. et al., Proc. Natl. Acad. USA, 63, 78-85 (1969)), Kabat numbering (Kabat, Sequences of Proteins of Immunological Interest (National Institutes of Health, Bethesda, M D, 1991, IMGT unique numbering (Lefranc M.-P. et al., IMGT unique numbering for immunoglobulin and T cell receptor constant domains and Ig superfamily C-like domains, Dev. Comp. Immunol., 29, 185-203 (2005), and IMGT exon numbering (Lefranc, supra).

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428 (EU numbering is used in this paragraph for the constant region) for increasing the half-life of an antibody. Substitution at any or all of positions 234, 235, 236 and/or 237 reduce affinity for Fcγ receptors, particularly FcγRI receptor (see, e.g., U.S. Pat. No. 6,624,821). An alanine substitution at positions 234, 235, and 237 of human IgG1 can be used for reducing effector functions. Some antibodies have alanine substitution at positions 234, 235 and 237 of human IgG1 for reducing effector functions. Optionally, positions 234, 236 and/or 237 in human IgG2 are substituted with alanine and position 235 with glutamine (see, e.g., U.S. Pat. No. 5,624,821). In some antibodies, a mutation at one or more of positions 241, 264, 265, 270, 296, 297, 322, 329, and 331 by EU numbering of human IgG1 is used. In some antibodies, a mutation at one or more of positions 318, 320, and 322 by EU numbering of human IgG1 is used. In some antibodies, positions 234 and/or 235 are substituted with alanine and/or position 329 is substituted with glycine. In some antibodies, positions 234 and 235 are substituted with alanine. In some antibodies, the isotype is human IgG2 or IgG4.

Antibodies can be expressed as tetramers containing two light and two heavy chains, as separate heavy chains, light chains, as Fab, Fab′, F(ab′)2, and Fv, or as single chain antibodies in which heavy and light chain mature variable domains are linked through a spacer.

Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of a one or more other isotypes. Thus, for example, another heavy chain constant region is of IgG1 G1m3 with or without the C-terminal lysine. Reference to a human constant region includes a constant region with any natural allotype or any permutation of residues occupying positions in natural allotypes. An exemplary heavy chain constant region is SEQ ID NO:176, with or without the C-terminal lysine, and an exemplary light chain constant region is SEQ ID NO:177.

### G. Expression of Recombinant Antibodies

A number of methods are known for producing chimeric and humanized antibodies using an antibody-expressing cell line (e.g., hybridoma). For example, the immunoglobulin variable regions of antibodies can be cloned and sequenced using well known methods. In one method, the heavy chain variable VH region is cloned by RT-PCR using mRNA prepared from hybridoma cells. Consensus primers are employed to the VH region leader peptide encompassing the translation initiation codon as the 5′ primer and a g2b constant regions specific 3′ primer. Exemplary primers are described in U.S. patent publication US 2005/0009150 by Schenk et al. (hereinafter “Schenk”). The sequences from multiple, independently derived clones can be compared to ensure no changes are introduced during amplification. The sequence of the VH region can also be determined or confirmed by sequencing a VH fragment obtained by 5′ RACE RT-PCR methodology and the 3′ g2b specific primer.

The light chain variable VL region can be cloned in an analogous manner. In one approach, a consensus primer set is designed for amplification of VL regions using a 5′ primer designed to hybridize to the VL region encompassing the translation initiation codon and a 3′ primer specific for the Ck region downstream of the V-J joining region. In a second approach, 5′RACE RT-PCR methodology is employed to clone a VL encoding cDNA. Exemplary primers are described in Schenk, supra. The cloned sequences are then combined with sequences encoding human (or other non-human species) constant regions.

In one approach, the heavy and light chain variable regions are re-engineered to encode splice donor sequences downstream of the respective VDJ or VJ junctions and are cloned into a mammalian expression vector, such as pCMV-hγ1 for the heavy chain and pCMV-Mcl for the light chain. These vectors encode human γ1 and Ck constant regions as exonic fragments downstream of the inserted variable region cassette. Following sequence verification, the heavy chain and light chain expression vectors can be co-transfected into CHO cells to produce chimeric antibodies. Conditioned media is collected 48 hours post-transfection and assayed by western blot analysis for antibody production or ELISA for antigen binding. The chimeric antibodies are humanized as described above.

Chimeric, veneered, humanized, and human antibodies are typically produced by recombinant expression. Recombinant polynucleotide constructs typically include an expression control sequence operably linked to the coding sequences of antibody chains, including naturally associated or heterologous expression control elements, such as a promoter. The expression control sequences can be promoter systems in vectors capable of transforming or transfecting eukaryotic or prokaryotic host cells. Once the vector has been incorporated into the appropriate host, the host is maintained under conditions suitable for high level expression of the nucleotide sequences and the collection and purification of the crossreacting antibodies.

These expression vectors are typically replicable in the host organisms either as episomes or as an integral part of the host chromosomal DNA. Commonly, expression vectors contain selection markers, e.g., ampicillin resistance or hygromycin resistance, to permit detection of those cells transformed with the desired DNA sequences.

E. coli is one prokaryotic host useful for expressing antibodies, particularly antibody fragments. Microbes, such as yeast, are also useful for expression. Saccharomyces is a yeast host with suitable vectors having expression control sequences, an origin of replication, termination sequences, and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells can be used for expressing nucleotide segments encoding immunoglobulins or fragments thereof. See Winnacker, From Genes to Clones, (VCH Publishers, N Y, 1987). A number of suitable host cell lines capable of secreting intact heterologous proteins have been developed, and include CHO cell lines, various COS cell lines, HeLa cells, HEK293 cells, L cells, and non-antibody-producing myelomas including Sp2/0 and NSO. The cells can be nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Expression control sequences can include promoters derived from endogenous genes, cytomegalovirus, SV40, adenovirus, bovine papillomavirus, and the like. See Co et al., J. Immunol. 148:1149 (1992).

Alternatively, antibody coding sequences can be incorporated in transgenes for introduction into the genome of a transgenic animal and subsequent expression in the milk of the transgenic animal (see, e.g., U.S. Pat. Nos. 5,741,957; 5,304,489; and 5,849,992). Suitable transgenes include coding sequences for light and/or heavy chains operably linked with a promoter and enhancer from a mammary gland specific gene, such as casein or beta lactoglobulin.

The vectors containing the DNA segments of interest can be transferred into the host cell by methods depending on the type of cellular host. For example, calcium chloride transfection is commonly utilized for prokaryotic cells, whereas calcium phosphate treatment, electroporation, lipofection, biolistics, or viral-based transfection can be used for other cellular hosts. Other methods used to transform mammalian cells include the use of polybrene, protoplast fusion, liposomes, electroporation, and microinjection. For production of transgenic animals, transgenes can be microinjected into fertilized oocytes or can be incorporated into the genome of embryonic stem cells or induced pluripotent stem cells (iPSCs), and the nuclei of such cells transferred into enucleated oocytes.

Having introduced vector(s) encoding antibody heavy and light chains into cell culture, cell pools can be screened for growth productivity and product quality in serum-free media. Top-producing cell pools can then be subjected of FACS-based single-cell cloning to generate monoclonal lines. Specific productivities above 50 pg or 100 pg per cell per day, which correspond to product titers of greater than 7.5 g/L culture, can be used. Antibodies produced by single cell clones can also be tested for turbidity, filtration properties, PAGE, IEF, UV scan, HP-SEC, carbohydrate-oligosaccharide mapping, mass spectrometry, and binding assay, such as ELISA or Biacore. A selected clone can then be banked in multiple vials and stored frozen for subsequent use.

Once expressed, antibodies can be purified according to standard procedures of the art, including protein A capture, HPLC purification, column chromatography, gel electrophoresis and the like (see generally, Scopes, Protein Purification (Springer-Verlag, NY, 1982)).

Methodology for commercial production of antibodies can be employed, including codon optimization, selection of promoters, selection of transcription elements, selection of terminators, serum-free single cell cloning, cell banking, use of selection markers for amplification of copy number, CHO terminator, or improvement of protein titers (see, e.g., U.S. Pat. Nos. 5,786,464; 6,114,148; 6,063,598; 7,569,339; WO2004/050884; WO2008/012142; WO2008/012142; WO2005/019442; WO2008/107388; WO2009/027471; and U.S. Pat. No. 5,888,809).

### H. Antibody Screening Assays

Antibodies can be initially screened for the intended binding specificity as described above. Active immunogens can likewise be screened for capacity to induce antibodies with such binding specificity. In this case, an active immunogen is used to immunize a laboratory animal and the resulting sera tested for the appropriate binding specificity.

Antibodies having the desired binding specificity can then be tested in cellular and animal models. The cells used for such screening are preferentially neuronal cells. A cellular model of tau pathology has been reported in which neuroblastoma cells are transfected with a four-repeat domain of tau, optionally with a mutation associated with tau pathology (e.g., delta K280, see Khlistunova, Current Alzheimer Research 4, 544-546 (2007)). In another model, tau is induced in the neuroblastoma N2a cell line by the addition of doxycyclin. The cell models enable one to study the toxicity of tau to cells in the soluble or aggregated state, the appearance of tau aggregates after switching on tau gene expression, the dissolution of tau aggregates after switching the gene expression off again, and the efficiency of antibodies in inhibiting formation of tau aggregates or disaggregating them.

Antibodies or active immunogens can also be screened in transgenic animal models of diseases associated with tau. Such transgenic animals can include a tau transgene (e.g., any of the human isoforms) and optionally a human APP transgene among others, such as a kinase that phosphorylates tau, ApoE, presenilin or alpha synuclein. Such transgenic animals are disposed to develop at least one sign or symptom of a disease associated with tau.

An exemplary transgenic animal is the K3 line of mice (Itner et al., Proc. Natl. Acad. Sci. USA 105(41):15997-6002 (2008)). These mice have a human tau transgene with a K 369 I mutation (the mutation is associated with Pick's disease) and a Thy 1.2 promoter. This model shows a rapid course of neurodegeneration, motor deficit and degeneration of afferent fibers and cerebellar granule cells. Another exemplary animal is the JNPL3 line of mice. These mice have a human tau transgene with a P301L mutation (the mutation is associated with frontotemporal dementia) and a Thy 1.2 promoter (Taconic, Germantown, N.Y., Lewis, et al., Nat Genet. 25:402-405 (2000)). These mice have a more gradual course of neurodegeneration. The mice develop neurofibrillary tangles in several brain regions and spinal cord, which is hereby incorporated by reference in its entirety). This is an excellent model to study the consequences of tangle development and for screening therapy that may inhibit the generation of these aggregates. Another advantage of these animals is the relatively early onset of pathology. In the homozygous line, behavioral abnormalities associated with tau pathology can be observed at least as early as 3 months, but the animals remain relatively healthy at least until 8 months of age. In other words, at 8 months, the animals ambulate, feed themselves, and can perform the behavioral tasks sufficiently well to allow the treatment effect to be monitored. Active immunization of these mice for 6-13 months with—AI wI KLH-PHF-1 generated titers of about 1,000 and showed fewer neurofibrillary tangles, less pSer422, and reduced weight loss relative to untreated control ice.

The activity of antibodies or active agents can be assessed by various criteria including reduction in amount of total tau or phosphorylated tau, reduction in other pathological characteristics, such as amyloid deposits of A3, and inhibition or delay or behavioral deficits. Active immunogens can also be tested for induction of antibodies in the sera. Both passive and active immunogens can be tested for passage of antibodies across the blood brain barrier into the brain of a transgenic animal. Antibodies or fragments inducing an antibody can also be tested in non-human primates that naturally or through induction develop symptoms of diseases characterized by tau. Tests on an antibody or active agent are usually performed in conjunction with a control in which a parallel experiment is conduct except that the antibody or active agent is absent (e.g., replaced by vehicle). Reduction, delay or inhibition of signs or symptoms disease attributable to an antibody or active agent under test can then be assessed relative to the control.

### I. Methods of Using the Antibodies of the Present Invention

The antibodies or antigen-binding fragments thereof described herein can inhibit or reduce internalization of tau by cells, inhibit or reduce tau induced toxicity, reduce or delay onset of behavioral deficit, inhibit or reduce levels of markers of tau pathology or inhibit or reduce development of tau pathology.

Also provided herein are methods of reducing internalization of tau by cells in a subject comprising administering to a subject in need thereof an amount of an antibody or an antigen-binding fragment thereof that reduces internalization of tau by cells, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO:14.

In some embodiments, administering the antibodies or antigen-binding fragments thereof described herein reduces internalization of tau by cells by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% (e.g., as compared to a level of tau internalization in the subject prior to administration or as compared to a level of tau internalization in a subject not administered the antibodies or antigen-binding fragments thereof). In some embodiments, administering the antibodies or antigen-binding fragments thereof described herein reduces internalization of tau by cells by about 10% to about 99%, about 20% to about 90%, about 30% to about 80%, about 40% to 80%, or about 50% to 75% (e.g., as compared to a level of tau internalization in the subject prior to administration or as compared to a level of tau internalization in a subject not administered the antibodies or antigen-binding fragments thereof)). In some embodiments, the administering results in about a 10% to about 99% reduction (e.g., about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% decrease) (e.g., as compared to a level of tau internalization in the subject prior to administration or as compared to a level of tau internalization in a subject not administered the antibodies or antigen-binding fragments thereof).

Also provided herein are methods of reducing tau induced toxicity in a subject comprising administering to a subject in need thereof an amount of an antibody or an antigen-binding fragment thereof that reduces tau induced toxicity, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO:14.

In some embodiments, administering the antibodies or antigen-binding fragments thereof described herein reduces tau induced toxicity by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% (e.g., as compared to a level of tau induced toxicity in the subject prior to administration or as compared to a level of tau induced toxicity in a subject not administered the antibodies or antigen-binding fragments thereof). In some embodiments, administering the antibodies or antigen-binding fragments thereof described herein reduces tau induced toxicity by about 10% to about 99%, about 20% to about 90%, about 30% to about 80%, about 40% to 80%, or about 50% to 75% (e.g., as compared to a level of tau induced toxicity in the subject prior to administration or as compared to a level of tau induced toxicity in a subject not administered the antibodies or antigen-binding fragments thereof)). In some embodiments, the administering results in about a 10% to about 99% reduction (e.g., about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% decrease) (e.g., as compared to a level of tau induced toxicity in the subject prior to administration or as compared to a level of tau induced toxicity in a subject not administered the antibodies or antigen-binding fragments thereof).

Also provided herein are methods of reducing or delaying onset of behavioral deficit in a subject comprising administering to a subject in need thereof an amount of an antibody or an antigen-binding fragment thereof that reduces or delays onset of behavioral deficit, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO: 14.

In some embodiments, administering the antibodies or antigen-binding fragments thereof described herein reduces or delays onset of behavioral deficit by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% (e.g., as compared to a level of behavioral deficit in the subject prior to administration or as compared to a level of behavioral deficit in a subject not administered the antibodies or antigen-binding fragments thereof). In some embodiments, administering the antibodies or antigen-binding fragments thereof described herein reduces or delays onset of behavioral deficit by about 10% to about 99%, about 20% to about 90%, about 30% to about 80%, about 40% to 80%, or about 50% to 75% (e.g., as compared to a level of behavioral deficit in the subject prior to administration or as compared to a level of behavioral deficit in a subject not administered the antibodies or antigen-binding fragments thereof)). In some embodiments, the administering results in about a 10% to about 99% reduction (e.g., about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% decrease) (e.g., as compared to a level of behavioral deficit in the subject prior to administration or as compared to a level of behavioral deficit in a subject not administered the antibodies or antigen-binding fragments thereof).

Also provided herein are methods of reducing levels of markers of tau pathology in a subject comprising administering to a subject in need thereof an amount of an antibody or an antigen-binding fragment thereof that reduces levels of markers of tau pathology, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO:14.

In some embodiments, administering the antibodies or antigen-binding fragments thereof described herein reduces levels of markers of tau pathology by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% (e.g., as compared to levels of markers of tau pathology in the subject prior to administration or as compared to levels of markers of tau pathology in a subject not administered the antibodies or antigen-binding fragments thereof). In some embodiments, administering the antibodies or antigen-binding fragments thereof described herein reduces levels of markers of tau pathology by about 10% to about 99%, about 20% to about 90%, about 30% to about 80%, about 40% to 80%, or about 50% to 75% (e.g., as compared to levels of markers of tau pathology in the subject prior to administration or as compared to levels of markers of tau pathology in a subject not administered the antibodies or antigen-binding fragments thereof)). In some embodiments, the administering results in about a 10% to about 99% reduction (e.g., about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% decrease) (e.g., as compared to levels of markers of tau pathology in the subject prior to administration or as compared to levels of markers of tau pathology in a subject not administered the antibodies or antigen-binding fragments thereof).

Also provided herein are methods of reducing development of tau pathology in a subject comprising administering to a subject in need thereof an amount of an antibody or an antigen-binding fragment thereof that reduces development of tau pathology, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO: 14.

In some embodiments, administering the antibodies or antigen-binding fragments thereof described herein reduces development of tau pathology by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or about 99% (e.g., as compared to a level of development of tau pathology in the subject prior to administration or as compared to a level of development of tau pathology in a subject not administered the antibodies or antigen-binding fragments thereof). In some embodiments, administering the antibodies or antigen-binding fragments thereof described herein reduces development of tau pathology by about 10% to about 99%, about 20% to about 90%, about 30% to about 80%, about 40% to 80%, or about 50% to 75% (e.g., as compared to a level of development of tau pathology in the subject prior to administration or as compared to a level of development of tau pathology in a subject not administered the antibodies or antigen-binding fragments thereof)). In some embodiments, the administering results in about a 10% to about 99% reduction (e.g., about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about a 20%, about a 10% to about a 15%, about a 15% to about a 99%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about a 20%, about a 20% to about a 99%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 99%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 99%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 99%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 99%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 99%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 99%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 99%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 99%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 99%, about a 65% to about a 95%, about a 65% to about a 90%, about a 65% to about a 85%, about a 65% to about a 80%, about a 65% to about a 75%, about a 65% to about a 70%, about a 70% to about a 99%, about a 70% to about a 95%, about a 70% to about a 90%, about a 70% to about a 85%, about a 70% to about a 80%, about a 70% to about a 75%, about a 75% to about a 99%, about a 75% to about a 95%, about a 75% to about a 90%, about a 75% to about a 85%, about a 75% to about a 80%, about a 80% to about a 99%, about a 80% to about a 95%, about a 80% to about a 90%, about a 80% to about a 85%, about a 85% to about a 99%, about a 85% to about a 95%, about a 85% to about a 90%, about a 90% to about a 99%, about a 90% to about a 95%, or about a 95% to about a 99% decrease) (e.g., as compared to a level of development of tau pathology in the subject prior to administration or as compared to a level of development of tau pathology in a subject not administered the antibodies or antigen-binding fragments thereof).

### IV. Patients Amenable to Treatment

The presence of neurofibrillary tangles has been found in several diseases including Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), and progressive supranuclear palsy (PSP). The present regimes can also be used in treatment or prophylaxis of any of these diseases. Because of the widespread association between neurological diseases and conditions and tau, the present regimes can be used in treatment or prophylaxis of any subject showing elevated levels of tau or phosphorylated tau (e.g., in the CSF) compared with a mean value in individuals without neurological disease. The present regimes can also be used in treatment or prophylaxis of neurological disease in individuals having a mutation in tau associated with neurological disease. The present methods are particularly suitable for treatment or prophylaxis of Alzheimer's disease, and especially in patients.

Patients amenable to treatment include individuals at risk of disease but not showing symptoms, as well as patients presently showing symptoms. Patients at risk of disease include those having a known genetic risk of disease. Such individuals include those having relatives who have experienced this disease, and those whose risk is determined by analysis of genetic or biochemical markers. Genetic markers of risk include mutations in tau, such as those discussed above, as well as mutations in other genes associated with neurological disease. For example, the ApoE4 allele in heterozygous and even more so in homozygous form is associated with risk of Alzheimer's disease. Other markers of risk of Alzheimer's disease include mutations in the APP gene, particularly mutations at position 717 and positions 670 and 671 referred to as the Hardy and Swedish mutations respectively, mutations in the presenilin genes, PS1 and PS2, a family history of AD, hypercholesterolemia or atherosclerosis. Individuals presently suffering from Alzheimer's disease can be recognized by PET imaging, from characteristic dementia, as well as the presence of risk factors described above. In addition, a number of diagnostic tests are available for identifying individuals who have AD. These include measurement of CSF tau or phospho-tau and Aβ42 levels. Elevated tau or phospho-tau and decreased Aβ42 levels signify the presence of AD. Some mutations associated with Parkinson's disease. Ala30Pro or Ala53, or mutations in other genes associated with Parkinson's disease such as leucine-rich repeat kinase, PARK8. Individuals can also be diagnosed with any of the neurological diseases mentioned above by the criteria of the DSM IV TR.

In asymptomatic patients, treatment can begin at any age (e.g., 10, 20, 30). Usually, however, it is not necessary to begin treatment until a patient reaches 40, 50, 60 or 70 years of age. Treatment typically entails multiple dosages over a period of time. Treatment can be monitored by assaying antibody levels over time. If the response falls, a booster dosage is indicated. In the case of potential Down's syndrome patients, treatment can begin antenatally by administering therapeutic agent to the mother or shortly after birth.

### V. Nucleic Acids

The invention further provides nucleic acids encoding any of the heavy and light chains described above (e.g., SEQ ID NO:7, SEQ ID NO:11, SEQ ID NOs:76-80, SEQ ID NOs:90-91, SEQ ID NOs:146-148, SEQ ID NOs:83-85, SEQ ID NOs:93-145, and SEQ ID NOs:178-181). An exemplary nucleic acid encoding a heavy chain of the invention is SEQ ID NO:182, and an exemplary nucleic acid encoding a light chain of the invention is SEQ ID NO:183. Optionally, such nucleic acids further encode a signal peptide and can be expressed with the signal peptide linked to the variable region. Coding sequences of nucleic acids can be operably linked with regulatory sequences to ensure expression of the coding sequences, such as a promoter, enhancer, ribosome binding site, transcription termination signal, and the like. The regulatory sequences can include a promoter, for example, a prokaryotic promoter or a eukaryotic promoter. The nucleic acids encoding heavy or light chains can be codon-optimized for expression in a host cell. The nucleic acids encoding heavy and light chains can encode a selectable gene. The nucleic acids encoding heavy and light chains can occur in isolated form or can be cloned into one or more vectors. The nucleic acids can be synthesized by, for example, solid state synthesis or PCR of overlapping oligonucleotides. Nucleic acids encoding heavy and light chains can be joined as one contiguous nucleic acid, e.g., within an expression vector, or can be separate, e.g., each cloned into its own expression vector.

### VI. Conjugated Antibodies

Conjugated antibodies that specifically bind to antigens, such as tau, are useful in detecting the presence of tau; monitoring and evaluating the efficacy of therapeutic agents being used to treat patients diagnosed with Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), or progressive supranuclear palsy (PSP); inhibiting or reducing aggregation of tau; inhibiting or reducing tau fibril formation; reducing or clearing tau deposits; stabilizing non-toxic conformations of tau; or treating or effecting prophylaxis of Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), or progressive supranuclear palsy (PSP) in a patient. For example, such antibodies can be conjugated with other therapeutic moieties, other proteins, other antibodies, and/or detectable labels. See WO 03/057838; U.S. Pat. No. 8,455,622. Such therapeutic moieties can be any agent that can be used to treat, combat, ameliorate, prevent, or improve an unwanted condition or disease in a patient, such as Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), or progressive supranuclear palsy (PSP).

Conjugated therapeutic moieties can include cytotoxic agents, cytostatic agents, neurotrophic agents, neuroprotective agents, radiotherapeutic agents, immunomodulators, or any biologically active agents that facilitate or enhance the activity of the antibody. A cytotoxic agent can be any agent that is toxic to a cell. A cytostatic agent can be any agent that inhibits cell proliferation. A neurotrophic agent can be any agent, including chemical or proteinaceous agents, that promotes neuron maintenance, growth, or differentiation. A neuroprotective agent can be agent, including chemical or proteinaceous agents, that protects neurons from acute insult or degenerative processes. An immunomodulator can be any agent that stimulates or inhibits the development or maintenance of an immunologic response. A radiotherapeutic agent can be any molecule or compound that emits radiation. If such therapeutic moieties are coupled to a tau-specific antibody, such as the antibodies described herein, the coupled therapeutic moieties will have a specific affinity for tau-related disease-affected cells over normal cells. Consequently, administration of the conjugated antibodies directly targets cancer cells with minimal damage to surrounding normal, healthy tissue. This can be particularly useful for therapeutic moieties that are too toxic to be administered on their own. In addition, smaller quantities of the therapeutic moieties can be used.

Some such antibodies can be modified to act as immunotoxins. See, e.g., U.S. Pat. No. 5,194,594. For example, ricin, a cellular toxin derived from plants, can be coupled to antibodies by using the bifunctional reagents S-acetylmercaptosuccinic anhydride for the antibody and succinimidyl 3-(2-pyridyldithio) propionate for ricin. See Pietersz et al., Cancer Res. 48(16):4469-4476 (1998). The coupling results in loss of B-chain binding activity of ricin, while impairing neither the toxic potential of the A-chain of ricin nor the activity of the antibody. Similarly, saporin, an inhibitor of ribosomal assembly, can be coupled to antibodies via a disulfide bond between chemically inserted sulfhydryl groups. See Polito et al., Leukemia 18:1215-1222 (2004).

Some such antibodies can be linked to radioisotopes. Examples of radioisotopes include, for example, yttrium⁹⁰ (90Y), indium¹¹¹ (111In), ¹³¹I, ⁹⁹mTc, radiosilver-111, radiosilver-199, and Bismuth²¹³. Linkage of radioisotopes to antibodies may be performed with conventional bifunction chelates. For radiosilver-111 and radiosilver-199 linkage, sulfur-based linkers may be used. See Hazra et al., Cell Biophys. 24-25:1-7 (1994). Linkage of silver radioisotopes may involve reducing the immunoglobulin with ascorbic acid. For radioisotopes such as 111In and 90Y, ibritumomab tiuxetan can be used and will react with such isotopes to form 111In-ibritumomab tiuxetan and 90Y-ibritumomab tiuxetan, respectively. See Witzig, Cancer Chemother. Pharmacol., 48 Suppl 1:S91-S95 (2001).

Some such antibodies can be linked to other therapeutic moieties. Such therapeutic moieties can be, for example, cytotoxic, cytostatic, neurotrophic, or neuroprotective. For example, antibodies can be conjugated with toxic chemotherapeutic drugs such as maytansine, geldanamycin, tubulin inhibitors such as tubulin binding agents (e.g., auristatins), or minor groove binding agents such as calicheamicin. Other representative therapeutic moieties include agents known to be useful for treatment, management, or amelioration of Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), or progressive supranuclear palsy (PSP).

Antibodies can also be coupled with other proteins. For example, antibodies can be coupled with Fynomers. Fynomers are small binding proteins (e.g., 7 kDa) derived from the human Fyn SH3 domain. They can be stable and soluble, and they can lack cysteine residues and disulfide bonds. Fynomers can be engineered to bind to target molecules with the same affinity and specificity as antibodies. They are suitable for creating multi-specific fusion proteins based on antibodies. For example, Fynomers can be fused to N-terminal and/or C-terminal ends of antibodies to create bi- and tri-specific FynomAbs with different architectures. Fynomers can be selected using Fynomer libraries through screening technologies using FACS, Biacore, and cell-based assays that allow efficient selection of Fynomers with optimal properties. Examples of Fynomers are disclosed in Grabulovski et al., J. Biol. Chem. 282:3196-3204 (2007); Bertschinger et al., Protein Eng. Des. Sel. 20:57-68 (2007); Schlatter et al., MAbs. 4:497-508 (2011); Banner et al., Acta. Crystallogr. D. Biol. Crystallogr. 69(Pt6):1124-1137 (2013); and Brack et al., Mol. Cancer Ther. 13:2030-2039 (2014).

The antibodies disclosed herein can also be coupled or conjugated to one or more other antibodies (e.g., to form antibody heteroconjugates). Such other antibodies can bind to different epitopes within tau or can bind to a different target antigen.

Antibodies can also be coupled with a detectable label. Such antibodies can be used, for example, for diagnosing Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), or progressive supranuclear palsy (PSP), and/or for assessing efficacy of treatment. Such antibodies are particularly useful for performing such determinations in subjects having or being susceptible to Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), or progressive supranuclear palsy (PSP), or in appropriate biological samples obtained from such subjects. Representative detectable labels that may be coupled or linked to an antibody include various enzymes, such as horseradish peroxidase, alkaline phosphatase, beta-galactosidase, or acetylcholinesterase; prosthetic groups, such streptavidin/biotin and avidin/biotin; fluorescent materials, such as umbelliferone, fluorescein, fluorescein isothiocyanate, rhodamine, dichlorotriazinylamine fluorescein, dansyl chloride or phycoerythrin; luminescent materials, such as luminol; bioluminescent materials, such as luciferase, luciferin, and aequorin; radioactive materials, such as radiosilver-111, radiosilver-199, Bismuth²¹³, iodine (¹³¹I, ¹²⁵I, ¹²³I, ¹²¹I) carbon (¹⁴C), sulfur (⁵S), tritium (³H), indium (¹¹⁵In, ¹¹³In, ¹¹²In, ¹¹¹In), technetium (⁹⁹Tc), thallium (²⁰¹Ti), gallium (⁶⁸Ga ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb ⁵¹Cr, ⁵⁴Mn, ⁷⁵Se, ¹¹³Sn, and ¹¹⁷Tin; positron emitting metals using various positron emission tomographies; nonradioactive paramagnetic metal ions; and molecules that are radiolabelled or conjugated to specific radioisotopes.

Linkage of radioisotopes to antibodies may be performed with conventional bifunction chelates. For radiosilver-111 and radiosilver-199 linkage, sulfur-based linkers may be used. See Hazra et al., Cell Biophys. 24-25:1-7 (1994). Linkage of silver radioisotopes may involve reducing the immunoglobulin with ascorbic acid. For radioisotopes such as 111In and 90Y, ibritumomab tiuxetan can be used and will react with such isotopes to form 111In-ibritumomab tiuxetan and 90Y-ibritumomab tiuxetan, respectively. See Witzig, Cancer Chemother. Pharmacol., 48 Suppl 1:S91-S95 (2001).

Therapeutic moieties, other proteins, other antibodies, and/or detectable labels may be coupled or conjugated, directly or indirectly through an intermediate (e.g., a linker), to an antibody of the invention. See e.g., Arnon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy,” in Monoclonal Antibodies For Cancer Detection And Therapy, Baldwin et al. (eds.), pp. 303-16 (Academic Press 1985); and Thorpe et al., Immunol. Rev., 62:119-58 (1982). Suitable linkers include, for example, cleavable and non-cleavable linkers. Different linkers that release the coupled therapeutic moieties, proteins, antibodies, and/or detectable labels under acidic or reducing conditions, on exposure to specific proteases, or under other defined conditions can be employed.

### VI. Pharmaceutical Compositions and Methods of Use

In prophylactic applications, an antibody or agent for inducing an antibody or a pharmaceutical composition the same is administered to a patient susceptible to, or otherwise at risk of a disease (e.g., Alzheimer's disease) in regime (dose, frequency and route of administration) effective to reduce the risk, lessen the severity, or delay the onset of at least one sign or symptom of the disease. In particular, the regime is preferably effective to inhibit or delay tau or phospho-tau and paired filaments formed from it in the brain, and/or inhibit or delay its toxic effects and/or inhibit/or delay development of behavioral deficits. In therapeutic applications, an antibody or agent to induce an antibody is administered to a patient suspected of, or already suffering from a disease (e.g., Alzheimer's disease) in a regime (dose, frequency and route of administration) effective to ameliorate or at least inhibit further deterioration of at least one sign or symptom of the disease. In particular, the regime is preferably effective to reduce or at least inhibit further increase of levels of tau, phosphor-tau, or paired filaments formed from it, associated toxicities and/or behavioral deficits. Behavioral deficits can be assessed from cognitive scales, such as ADAS Cog, or the mini-mental status exam. Treatment can be evidenced by improvement on these scales optionally to within normal range, reduced decline or maintaining a constant value on the scales. Prophylaxis can be evidenced by reduced or delayed or lack of decline on these scales. Treatment and prophylaxis can also be evidenced by changes in the levels of one or more markers including those disclosed in the examples

A regime is considered therapeutically or prophylactically effective if an individual treated patient achieves an outcome more favorable than the mean outcome in a control population of comparable patients not treated by methods of the invention, or if a more favorable outcome is demonstrated in treated patients versus control patients in a controlled clinical trial (e.g., a phase II, phase 11/III or phase III trial) at the p<0.05 or 0.01 or even 0.001 level.

Effective doses of vary depending on many different factors, such as means of administration, target site, physiological state of the patient, whether the patient is an ApoE carrier, whether the patient is human or an animal, other medications administered, and whether treatment is prophylactic or therapeutic.

Exemplary dosage ranges for antibodies are from about 0.01 to 60 mg/kg, or from about 0.1 to 3 mg/kg or 0.15-2 mg/kg or 0.15-1.5 mg/kg, of patient body weight. Antibody can be administered such doses daily, on alternative days, weekly, fortnightly, monthly, quarterly, or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months.

The amount of an agent for active administration varies from 0.1-500 μg per patient and more usually from 1-100 or 1-10 μg per injection for human administration. The timing of injections can vary significantly from once a day, to once a year, to once a decade. A typical regimen consists of an immunization followed by booster injections at time intervals, such as 6 week intervals or two months. Another regimen consists of an immunization followed by booster injections 1, 2 and 12 months later. Another regimen entails an injection every two months for life. Alternatively, booster injections can be on an irregular basis as indicated by monitoring of immune response.

Antibodies or agents for inducing antibodies are preferably administered via a peripheral route (i.e., one in which an administered or induced antibody crosses the blood brain barrier to reach an intended site in the brain. Routes of administration include topical, intravenous, oral, subcutaneous, intraarterial, intracranial, intrathecal, intraperitoneal, intranasal, intraocular, or intramuscular. Preferred routes for administration of antibodies are intravenous and subcutaneous. Preferred routes for active immunization are subcutaneous and intramuscular. This type of injection is most typically performed in the arm or leg muscles. In some methods, agents are injected directly into a particular tissue where deposits have accumulated, for example intracranial injection.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, antibodies can be in lyophilized form for constitution with a suitable vehicle, e.g., sterile pyrogen-free water, before use.

The present regimes can be administered in combination with another agent effective in treatment or prophylaxis of the disease being treated. For example, in the case of Alzheimer's disease, the present regimes can be combined with immunotherapy against A3 (WO/2000/072880), cholinesterase inhibitors or memantine or in the case of Parkinson's disease immunotherapy against alpha synuclein WO/2008/103472, Levodopa, dopamine agonists, COMT inhibitors, MAO-B inhibitors, Amantadine, or anticholinergic agents.

Antibodies are administered in an effective regime meaning a dosage, route of administration and frequency of administration that delays the onset, reduces the severity, inhibits further deterioration, and/or ameliorates at least one sign or symptom of a disorder being treated. If a patient is already suffering from a disorder, the regime can be referred to as a therapeutically effective regime. If the patient is at elevated risk of the disorder relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients.

Exemplary dosages for an antibody are 0.1-60 mg/kg (e.g., 0.5, 3, 10, 30, or 60 mg/kg), or 0.5-5 mg/kg body weight (e.g., 0.5, 1, 2, 3, 4 or 5 mg/kg) or 10-4000 mg or 10-1500 mg as a fixed dosage. The dosage depends on the condition of the patient and response to prior treatment, if any, whether the treatment is prophylactic or therapeutic and whether the disorder is acute or chronic, among other factors.

Administration can be parenteral, intravenous, oral, subcutaneous, intra-arterial, intracranial, intrathecal, intraperitoneal, topical, intranasal or intramuscular. Some antibodies can be administered into the systemic circulation by intravenous or subcutaneous administration. Intravenous administration can be, for example, by infusion over a period such as 30-90 min.

The frequency of administration depends on the half-life of the antibody in the circulation, the condition of the patient and the route of administration among other factors. The frequency can be daily, weekly, monthly, quarterly, or at irregular intervals in response to changes in the patient's condition or progression of the disorder being treated. An exemplary frequency for intravenous administration is between weekly and quarterly over a continuous cause of treatment, although more or less frequent dosing is also possible. For subcutaneous administration, an exemplary dosing frequency is daily to monthly, although more or less frequent dosing is also possible.

The number of dosages administered depends on whether the disorder is acute or chronic and the response of the disorder to the treatment. For acute disorders or acute exacerbations of a chronic disorder, between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, an antibody can be administered at regular intervals, e.g., weekly, fortnightly, monthly, quarterly, every six months for at least 1, 5 or 10 years, or the life of the patient.

### A. Diagnostics and Monitoring Methods

### In Vivo Imaging, Diagnostic Methods, and Optimizing Immunotherapy

The invention provides methods of in vivo imaging tau protein deposits (e.g., neurofibrillary tangles and tau inclusions) in a patient. The methods work by administering a reagent, such as antibody that binds tau (e.g., a mouse, humanized, chimeric or veneered 3D6 antibody), to the patient and then detecting the agent after it has bound. Antibodies binding to an epitope of tau within amino acid residues 199-213 or 262-276 of SEQ ID NO:3 (corresponding to amino acid residues 257-271 or 320-334, respectively, of SEQ ID NO:1) or within amino acid residues 259-268 or 290-299 or 321-330 or 353-362 of SEQ ID NO:1, are preferred. In some methods, the antibody binds to an epitope within amino acid residues 199-213 of SEQ ID NO:3 (corresponding to amino acid residues 257-271 of SEQ ID NO:1), or within amino acids 262-276 of SEQ ID NO:3 (corresponding to amino acid residues 320-334 of SEQ ID NO:1). In some methods, the antibody binds to an epitope within amino acid residues 259-268 of SEQ ID NO:1, within amino acids 290-299 of SEQ ID NO:1, within amino acids 321-330 of SEQ ID NO1, or within amino acids 353-362 of SEQ ID NO:1. A clearing response to the administered antibodies can be avoided or reduced by using antibody fragments lacking a full-length constant region, such as Fabs. In some methods, the same antibody can serve as both a treatment and diagnostic reagent.

Diagnostic reagents can be administered by intravenous injection into the body of the patient, or directly into the brain by intracranial injection or by drilling a hole through the skull. The dosage of reagent should be within the same ranges as for treatment methods. Typically, the reagent is labeled, although in some methods, the primary reagent with affinity for tau is unlabeled and a secondary labeling agent is used to bind to the primary reagent. The choice of label depends on the means of detection. For example, a fluorescent label is suitable for optical detection. Use of paramagnetic labels is suitable for tomographic detection without surgical intervention. Radioactive labels can also be detected using positron emission tomography (PET) or single-photon emission computed tomography (SPECT).

The methods of in vivo imaging of tau protein deposits are useful to diagnose or confirm diagnosis of a tauopathy, such as Alzheimer's disease, frontotemporal lobar degeneration, progressive supranuclear palsy and Pick's disease, or susceptibility to such a disease. For example, the methods can be used on a patient presenting with symptoms of dementia. If the patient has abnormal neurofibrillary tangles, then the patient is likely suffering from Alzheimer's disease. Alternatively, if the patient has abnormal tau inclusions, then depending on the location of the inclusions, the patient may be suffering from frontotemporal lobar degeneration. The methods can also be used on asymptomatic patients. Presence of abnormal tau protein deposits indicates susceptibility to future symptomatic disease. The methods are also useful for monitoring disease progression and/or response to treatment in patients who have been previously diagnosed with a tau-related disease.

Diagnosis can be performed by comparing the number, size, and/or intensity of labeled loci, to corresponding baseline values. The base line values can represent the mean levels in a population of undiseased individuals. Baseline values can also represent previous levels determined in the same patient. For example, baseline values can be determined in a patient before beginning tau immunotherapy treatment, and measured values thereafter compared with the baseline values. A decrease in values relative to baseline signals a positive response to treatment.

In some patients, diagnosis of a tauopathy may be aided by performing a PET scan. A PET scan can be performed using, for example, a conventional PET imager and auxiliary equipment. The scan typically includes one or more regions of the brain known in general to be associated with tau protein deposits and one or more regions in which few if any deposits are generally present to serve as controls.

The signal detected in a PET scan can be represented as a multidimensional image. The multidimensional image can be in two dimensions representing a cross-section through the brain, in three dimensions, representing the three dimensional brain, or in four dimensions representing changes in the three dimensional brain over time. A color scale can be used with different colors indicating different amounts of label and, inferentially, tau protein deposit detected. The results of the scan can also be presented numerically, with numbers relating to the amount of label detected and consequently amount of tau protein deposits. The label present in a region of the brain known to be associated with deposits for a particular tauopathy (e.g., Alzheimer's disease) can be compared with the label present in a region known not to be associated with deposits to provide a ratio indicative of the extent of deposits within the former region. For the same radiolabeled ligand, such ratios provide a comparable measure of tau protein deposits and changes thereof between different patients.

In some methods, a PET scan is performed concurrent with or in the same patient visit as an MRI or CAT scan. An MRI or CAT scan provides more anatomical detail of the brain than a PET scan. However, the image from a PET scan can be superimposed on an MRI or CAT scan image more precisely indicating the location of PET ligand and inferentially tau deposits relative to anatomical structures in the brain. Some machines can perform both PET scanning and MRI or CAT scanning without the patient changing positions between the scans facilitating superimposition of images.

Suitable PET ligands include radiolabeled antibodies of the invention (e.g., a mouse, humanized, chimeric or veneered 3D6 antibody). The radioisotope used can be, for example, C”, N¹³, O¹⁵, F¹⁸, or I¹²³. The interval between administering the PET ligand and performing the scan can depend on the PET ligand and particularly its rate of uptake and clearing into the brain, and the half-life of its radiolabel.

PET scans can also be performed as a prophylactic measure in asymptomatic patients or in patients who have symptoms of mild cognitive impairment but have not yet been diagnosed with a tauopathy but are at elevated risk of developing a tauopathy. For asymptomatic patients, scans are particularly useful for individuals considered at elevated risk of tauopathy because of a family history, genetic or biochemical risk factors, or mature age. Prophylactic scans can commence for example, at a patient age between 45 and 75 years. In some patients, a first scan is performed at age 50 years.

Prophylactic scans can be performed at intervals of for example, between six months and ten years, preferably between 1-5 years. In some patients, prophylactic scans are performed annually. If a PET scan performed as a prophylactic measure indicates abnormally high levels of tau protein deposits, immunotherapy can be commenced and subsequent PET scans performed as in patients diagnosed with a tauopathy. If a PET scanned performed as a prophylactic measure indicates levels of tau protein deposits within normal levels, further PET scans can be performed at intervals of between six months and 10 years, and preferably 1-5 years, as before, or in response to appearance of signs and symptoms of a tauopathy or mild cognitive impairment. By combining prophylactic scans with administration of tau-directed immunotherapy if and when an above normal level of tau protein deposits is detected, levels of tau protein deposits can be reduced to, or closer to, normal levels, or at least inhibited from increasing further, and the patient can remain free of the tauopathy for a longer period than if not receiving prophylactic scans and tau-directed immunotherapy (e.g., at least 5, 10, 15 or 20 years, or for the rest of the patient's life).

Normal levels of tau protein deposits can be determined by the amount of neurofibrillary tangles or tau inclusions in the brains of a representative sample of individuals in the general population who have not been diagnosed with a particular tauopathy (e.g., Alzheimer's disease) and are not considered at elevated risk of developing such disease (e.g., a representative sample of disease-free individuals under 50 years of age). Alternatively, a normal level can be recognized in an individual patient if the PET signal according to the present methods in a region of the brain in which tau protein deposits are known to develop is not different (within the accuracy of measurement) from the signal from a region of the brain in which it is known that such deposits do not normally develop. An elevated level in an individual can be recognized by comparison to the normal levels (e.g., outside mean and variance of a standard deviation) or simply from an elevated signal beyond experimental error in a region of the brain associated with tau protein deposits compared with a region not known to be associated with deposits. For purposes of comparing the levels of tau protein deposits in an individual and population, the tau protein deposits should preferably be determined in the same region(s) of the brain, these regions including at least one region in which tau protein deposits associated with a particular tauopathy (e.g., Alzheimer's disease) are known to form. A patient having an elevated level of tau protein deposits is a candidate for commencing immunotherapy.

After commencing immunotherapy, a decrease in the level of tau protein deposits can be first seen as an indication that the treatment is having the desired effect. The observed decrease can be, for example, in the range of 1-100%, 1-50%, or 1-25% of the baseline value. Such effects can be measured in one or more regions of the brain in which deposits are known to form or can be measured from an average of such regions. The total effect of treatment can be approximated by adding the percentage reduction relative to baseline to the increase in tau protein deposits that would otherwise occur in an average untreated patient.

Maintenance of tau protein deposits at an approximately constant level or even a small increase in tau protein deposits can also be an indication of response to treatment albeit a suboptimal response. Such responses can be compared with a time course of levels of tau protein deposits in patients with a particular tauopathy (e.g., Alzheimer's disease) that did not receive treatment, to determine whether the immunotherapy is having an effect in inhibiting further increases of tau protein deposits.

Monitoring of changes in tau protein deposits allows adjustment of the immunotherapy or other treatment regime in response to the treatment. PET monitoring provides an indication of the nature and extent of response to treatment. Then a determination can be made whether to adjust treatment and if desired treatment can be adjusted in response to the PET monitoring. PET monitoring thus allows for tau-directed immunotherapy or other treatment regime to be adjusted before other biomarkers, MRI or cognitive measures have detectably responded. A significant change means that comparison of the value of a parameter after treatment relative to basement provides some evidence that treatment has or has not resulted in a beneficial effect. In some instances, a change of values of a parameter in a patient itself provides evidence that treatment has or has not resulted in a beneficial effect. In other instances, the change of values, if any, in a patient, is compared with the change of values, if any, in a representative control population of patients not undergoing immunotherapy. A difference in response in a particular patient from the normal response in the control patient (e.g., mean plus variance of a standard deviation) can also provide evidence that an immunotherapy regime is or is not achieving a beneficial effect in a patient.

In some patients, monitoring indicates a detectable decline in tau protein deposits but that the level of tau protein deposits remains above normal. In such patients, if there are no unacceptable side effects, the treatment regime can be continued as is or even increased in frequency of administration and/or dose if not already at the maximum recommended dose.

If the monitoring indicates levels of tau protein deposits in a patient have already been reduced to normal, or near-normal, levels of tau protein deposits, the immunotherapy regime can be adjusted from one of induction (i.e., that reduces the level of tau protein deposits) to one of maintenance (i.e. that maintains tau protein deposits at an approximately constant level). Such a regime can be affected by reducing the dose and or frequency of administering immunotherapy.

In other patients, monitoring can indicate that immunotherapy is having some beneficial effect but a suboptimal effect. An optimal effect can be defined as a percentage reduction in the level of tau protein deposits within the top half or quartile of the change in tau protein deposits (measured or calculated over the whole brain or representative region(s) thereof in which tau protein deposits are known to form) experienced by a representative sample of tauopathy patients undergoing immunotherapy at a given time point after commencing therapy. A patient experiencing a smaller decline or a patient whose tau protein deposits remains constant or even increases, but to a lesser extent than expected in the absence of immunotherapy (e.g., as inferred from a control group of patients not administered immunotherapy) can be classified as experiencing a positive but suboptimal response. Such patients can optionally be subject to an adjustment of regime in which the dose and or frequency of administration of an agent is increased.

In some patients, tau protein deposits may increase in similar or greater fashion to tau deposits in patients not receiving immunotherapy. If such increases persist over a period of time, such as 18 months or 2 years, even after any increase in the frequency or dose of agents, immunotherapy can if desired be discontinued in favor of other treatments.

The foregoing description of diagnosing, monitoring, and adjusting treatment for tauopathies has been largely focused on using PET scans. However, any other technique for visualizing and/or measuring tau protein deposits that is amenable to the use of tau antibodies of the invention (e.g., a mouse, humanized, chimeric or veneered 3D6 antibody) can be used in place of PET scans to perform such methods.

Also provided are methods of detecting an immune response against tau in a patient suffering from or susceptible to diseases associated with tau. The methods can be used to monitor a course of therapeutic and prophylactic treatment with the agents provided herein. The antibody profile following passive immunization typically shows an immediate peak in antibody concentration followed by an exponential decay. Without a further dose, the decay approaches pretreatment levels within a period of days to months depending on the half-life of the antibody administered. For example, the half-life of some human antibodies is of the order of 20 days.

In some methods, a baseline measurement of antibody to tau in the subject is made before administration, a second measurement is made soon thereafter to determine the peak antibody level, and one or more further measurements are made at intervals to monitor decay of antibody levels. When the level of antibody has declined to baseline or a predetermined percentage of the peak less baseline (e.g., 50%, 25% or 10%), administration of a further dose of antibody is administered. In some methods, peak or subsequent measured levels less background are compared with reference levels previously determined to constitute a beneficial prophylactic or therapeutic treatment regime in other subjects. If the measured antibody level is significantly less than a reference level (e.g., less than the mean minus one or, preferably, two standard deviations of the reference value in a population of subjects benefiting from treatment) administration of an additional dose of antibody is indicated.

Also provided are methods of detecting tau in a subject, for example, by measuring tau in a sample from a subject or by in vivo imaging of tau in a subject. Such methods are useful to diagnose or confirm diagnosis of diseases associated with tau, or susceptibility thereto. The methods can also be used on asymptomatic subjects. The presence of tau indicates susceptibility to future symptomatic disease. The methods are also useful for monitoring disease progression and/or response to treatment in subjects who have been previously diagnosed with Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), or progressive supranuclear palsy (PSP).

Biological samples obtained from a subject having, suspected of having, or at risk of having Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), or progressive supranuclear palsy (PSP) can be contacted with the antibodies disclosed herein to assess the presence of tau. For example, levels of tau in such subjects may be compared to those present in healthy subjects. Alternatively, levels of tau in such subjects receiving treatment for the disease may be compared to those of subjects who have not been treated for Alzheimer's disease, Down's syndrome, mild cognitive impairment, primary age-related tauopathy, postencephalitic parkinsonism, posttraumatic dementia or dementia pugilistica, Pick's disease, type C Niemann-Pick disease, supranuclear palsy, frontotemporal dementia, frontotemporal lobar degeneration, argyrophilic grain disease, globular glial tauopathy, amyotrophic lateral sclerosis/parkinsonism dementia complex of Guam, corticobasal degeneration (CBD), dementia with Lewy bodies, Lewy body variant of Alzheimer disease (LBVAD), chronic traumatic encephalopathy (CTE), globular glial tauopathy (GGT), or progressive supranuclear palsy (PSP). Some such tests involve a biopsy of tissue obtained from such subjects. ELISA assays may also be useful methods, for example, for assessing tau in fluid samples.

### VIII. Kits

The invention further provides kits (e.g., containers) comprising an antibody disclosed herein and related materials, such as instructions for use (e.g., package insert). The instructions for use may contain, for example, instructions for administration of the antibody and optionally one or more additional agents. The containers of antibody may be unit doses, bulk packages (e.g., multi-dose packages), or sub-unit doses.

Package insert refers to instructions customarily included in commercial packages of therapeutic products that contain information about the indications, usage, dosage, administration, contraindications and/or warnings concerning the use of such therapeutic products

Kits can also include a second container comprising a pharmaceutically-acceptable buffer, such as bacteriostatic water for injection (BWFI), phosphate-buffered saline, Ringer's solution and dextrose solution. It can also include other materials desirable from a commercial and user standpoint, including other buffers, diluents, filters, needles, and syringes.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLES

### Example 1: Mouse 3D6 and Humanized Variant hu3D6VHv1bA11/L2-DIM4 Block Internalization of Tau

An internalization assay employing fluorescence activated cell sorting (FACS) was performed to evaluate the ability of various antibodies to block neuronal internalization of tau. Antibodies that block internalization will likely block transmission of tau.

Soluble tau aggregates were generated by incubation of recombinant full length tau with equimolar amounts of low molecular weight heparin for 3d at 37° C. After incubation, insoluble and soluble tau was separated by centrifugation at 10,000×g for 15 minutes. The supernatant was then resolved by preparative size exclusion chromatography, and aggregate peaks (greater than 100 kDa) were collected and concentrated. To measure internalization, the soluble aggregate fraction was labeled with pHrodo Red succinimidyl ester, which fluoresces when internalized into the endolysosomal pathway.

pHrodo-labeled 4RON human tau P301L soluble oligomer (1.5 μg/ml final concentration) was preincubated with anti-tau antibodies (dose titration: 80 μg/ml starting concentration followed by 4-fold serial dilutions) for 30 min at room temperature in cell culture media. Tau/antibody mixture was then added to B103 neuroblastoma cell lines at 500,000 cells/ml final concentration and incubated for 3-4 hrs at 37° C. in a tissue culture incubator (5% CO₂). Cells were then washed 3× with culture media, followed by 10 minutes culture media incubation, and washed 2× with FACS buffer (1% FBS in PBS). Cells were resuspended in 100 μl FACS buffer and Texas Red mean fluorescence intensity measured by FACS LSR II. Texas red fluorescence from pHrodo is activated by low pH associated with endolysosomal compartments upon internalization. Because FACS detects cells and pHrodo only fluoresces upon internalization, only tau internalized by the cells will be detected. The lower the mean fluorescence intensity, the lower the amount of internalized tau, which suggests a higher blocking activity of the antibody tested.

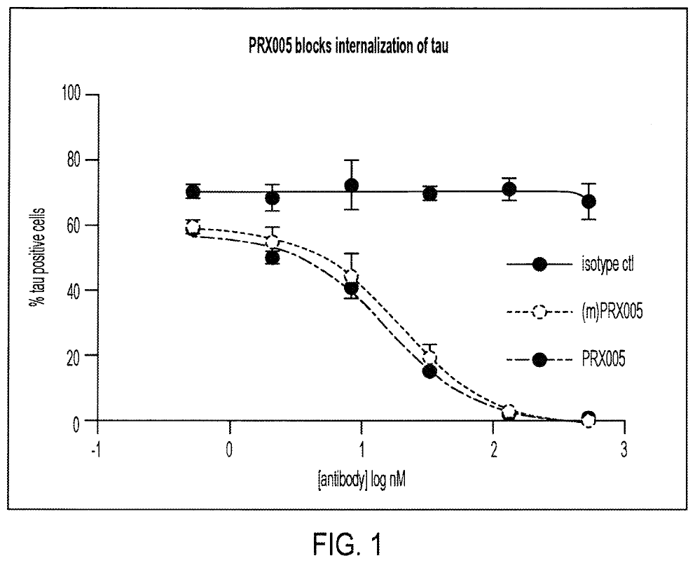

Both (m)PRX005 (mouse 3D6) and PRX005 (hu3D6VHv1bA11/L2-DIM4) displayed a high degree of inhibitory activity in the model of tau internalization at an equivalent concentration compared to isotype control (isotype ctl) (FIG. 1). All values are mean±SD (n=3-5). The IC₅₀=9 nM. [tau]=167 nM.

### Example 2 Mouse 3D6 Reduces Pathological Tau Development in an Induced Tau Seeding Model with Alzheimer's Disease Extracts

The ability of mouse 3D6 to reduce pathological tau development was investigated in an induced tau seeding model with Alzheimer's disease extracts.

Mice expressing the clinical mutant of human Tau (P301S) under control of the mouse PrP promoter (neuron-specific expression) were utilized in this study, before the appearance of promoter-driven tau pathology. hTauP301S mice display filamentous neuritic tau lesions by 6 months of age, which progressively accumulate in association with neuronal loss and hippocampal and entorhinal cortical atrophy by 9-12 months of age. Mice (average 3 month old) underwent a single stereotaxic injection into the hippocampus. Starting 7 days before hippocampal injection, mice (n=30/group) received weekly IP (intraperitoneal) injections (50 mg/kg) for 2 months of mouse 3D6 (mPRX005)-IgG2a or an IgG2a negative control antibody. At the end of the study, the extent of pathological progression was assessed with AT8 antibody.

AD Brain Extract Preparation

Alzheimer's disease tissue was homogenized and Tau protein was enriched by first resuspending human gray matter 9 volume equivalents (to original mass of brain tissue) in Buffer A (10 mM TRIS 0.8 M NaCl, 10% sucrose, 2 mM DTT, 1 mM EGTA pH 7.4) using 20 strokes of a motorized Dounce homogenizer. This homogenate was then centrifuged at 10,000×g for 10 minutes at 4° C. The supernatant was filtered through a Kimwipe and kept on ice until further use. The pellet from the centrifugation step was resuspended again in 9 volume equivalents and centrifuged at the same settings as before. The supernatant from this centrifugation was again filtered through a Kimwipe and combined with the other fraction. These pooled supernatants were then adjusted to 1% lauryl sarcosine (using a 30% stock) and stirred at 180 RPM for 180 minutes at room temperature. This lysate was then centrifuged at 250,000×g for 90 minutes 4° C. The supernatant was kept as the sarkosyl soluble fraction and the pellet was gently washed with 6 mL of PBS such that it would not dislodge from the tube. The wash was removed, and another 2 mL wash was applied to the pellet. After removing this wash, the pellet was dislodged using 1 mL of PBS, resuspended, and transferred to a clean and sterile microcentrifuge tube. The resuspended pellet was now centrifuged again at 250,000×g for 30 minutes at 4° C. After centrifugation, the pellet was separated from the supernatant and the pellet was resuspended in 0.1 mL PBS/g starting weight. The pellet was broken up via pipette tip and rotated end-over-end for 16 hours at room temperature. After incubation, the resuspension was sonicated for fifteen 0.5s pulses (set at 15% power and 100% duty cycle) using a tip probe sonicator. The sonicated material was then passed through a 27G needle and rotated for 30 minutes end-over-end at room temperature. The solution was sonicated, and the sample was centrifuged at 100,000×g for 30 minutes at 4° C. The supernatant was kept as the high g supernatant fraction and the pellet was resuspended in PBS using 50 uL/g original material. The resuspended pellet was sonicated at 20% power for one-hundred 0.5s pulses with a 30 s rest on ice every 20 pulses. This homogenate is centrifuged at 10,000×g for 10 minutes at 4° C. This final supernatant is kept as the enriched sarkosyl insoluble Tau protein fraction and the pellet was discarded.

Dose Formulation and Administration

Preparation of Substances

The needed amount of sarkosyl-enriched brain fraction from AD patients was defrosted and sonicated for 3 minutes of sonication with 10% power and 10s on 5s off pulse pattern in a sonicator water bath (QSonica) filled with ice water. 1 μl of antibody mouse 3D6 (mPRX005), or 6F10 (control antibody) (all without dilution at 10 mg/ml) or PBS, were mixed by pipetting with 1 μl of the sarkosyl-enriched AD brain before stereotactic injection right, after sonication. Murine IgG2a promotes faster tau clearance by phagocytes in vitro compared to IgG1 and was used in this study. IgG2a mPRX005 (mouse 3D6) showed superior efficacy versus IgG1 in vivo.

Before the study, the test and control articles were formulated in sterile vehicle of 1× phosphate-buffered saline (1×PBS) to a concentration of 5 mg/mL, to allow administration at the dose volume of 10 mL/kg.

Stereotaxic Injections

Mice were anesthetized using isoflurane and placed flat skull in a stereotaxic apparatus (Kopf instruments). The surgery area was shaved and disinfected using 70% alcohol and iodine, and an incision was made in the skin. A hole was drilled in the skull at the correct rostral and lateral position with respect to bregma (coordinates described in Table 3) using a micro drill and a drill bit with a head diameter of 0.9 mm. A 30-gauge cannula kept in a holder was lowered in position (coordinates described in table 3). The pre-incubated AD brain extracts (1 μl AD brain extract) were injected at a speed of 1 l/min (WPI, AL-1000, infusion pump). The injection volume was infused via PE10 tubing attached to a gastight 10-1 Hamilton syringe (#1701) placed in an infusion/withdrawal pump. After infusion, the needle was left in position for 5 min then slowly withdrawn. The skin incision was closed with sutures. Carprofen was injected s.c. as analgesia. Body temperature of the mice was maintained during the whole procedure, until mice recovered from anesthesia, by using a heating pad.

TABLE 3
Experimental Design

| | |
|---|---|
| Administration route | Intracranial stereotaxic injection |
| Targeted tissue | Hippocampus, CA1 |
| Stereotaxic | A/P: −1.7 mm |
| coordinates | M/L: −1.9 mm |
| | D/V: −2.0 mm |
| Dosing volume | 1 μl for pre-incubated brainstem |
| | homogenates |
| Injection speed | 1 μl/min |
| Frequency of | Single dose |
| treatment | |
| Dosing logs | Dosing logs were maintained for each |
| | mouse to serve as a safeguard against |
| | double or mismatched dosing. |

A/P = antero-posterior;
L = medial-lateral;
D/V = Dorso-ventral

Sample Collection and Processing

Mice were sacrificed at 5 months of age (2 months after stereotaxic injection) using CO₂ and flushed trans-cardially with ice-cold 1×PBS for 5 minutes (3 ml/min via peristaltic pump) via the left ventricle. The right atrium was cut as an outflow route. The brain was removed from the cranium. The whole brain was fixed in 10% neutral buffered formalin (NBF) for 24h and stored in 1×PBS at 4° C. until further processing.

Histological Staining

Reagents for immunofluorescence staining were supplied as described in Table 4.

TABLE 4
Immunohistochemistry Reagent Information

| Reagent | Host | Conjugation | Manufacturer | Cat No. | Dilution |
|---|---|---|---|---|---|
| AT8 | Mouse | Biotin | Thermo | MN1020B | 1:5000 |
| | | | Scientific | | |
| VECTASTAIN ® | N/A | N/A | Vector | PK-6100 | 1:100 |
| Elite ABC (avidin- | | | | | |
| biotin-HRP complex) | | | | | |

Brains were sent to Neuroscience Associate (Knoxville, TN), treated overnight with 20% glycerol and 2% dimethylsulfoxide to prevent freeze-artifacts. The specimens were then embedded in a gelatin matrix using MultiBrain®/MultiCord® Technology (NeuroScience Associates, Knoxville, TN). The blocks were rapidly frozen, after curing by immersion in 2-Methylbutane chilled with crushed dry ice and mounted on a freezing stage of an AO 860 sliding microtome. The MultiBrain®/MultiCord® blocks were sectioned in coronally at 35 obtaining sections containing the hippocampus (bregma −0.5 and −4.0). All sections were cut through the entire length of the specimen segment and collected sequentially into series of 24 containers. All containers contained Antigen Preserve solution (50% PBS pH7.0, 50% Ethylene Glycol, 1% Polyvinyl Pyrrolidone). For immunohistochemistry, free floating sections were stained with AT8 (1:5000, Thermo Scientific). All incubation solutions from the blocking serum onward use Tris buffered saline (TBS) with Triton X-100 as the vehicle; all rinses are with TBS. After a hydrogen peroxide treatment and blocking serum, the sections were immunostained with biotinylated AT8 (1:5000) overnight at room temperature. Vehicle solutions contained Triton X-100 for permeabilization. Following rinses, Vector Lab's ABC solution (avidin-biotin-HRP complex; VECTASTAIN® Elite ABC, Vector, Burlingame, CA) was applied. The sections were again rinsed, then treated with diaminobenzidine tetrahydrochloride (DAB) and hydrogen peroxide to create a visible reaction product. Following further rinses, the sections were mounted on gelatin coated glass slides, air dried. The slides were dehydrated in alcohols, cleared in xylene and coverslipped. Each slide was laser etched with the block number and the stain. Following serial ordering of slides, rostral to caudal for each stain, the slides were numbered by permanent ink in the upper right corner and digitally scanned at 10× on a Huron Digital Pathology Tissuescope LE 120.

Immunohistochemical Analysis

A8 positive neurons in the ipsilateral and contralateral hippocampi (cornu ammonis, dentate gyrus and subiculum) were quantified using a particle counter function in Image J. A total of 15 sections spaced at 210 m intervals were quantified. Only neurons bigger than 5 m with a distinguishable nucleus and neuronal projections were included.

Two-way ANOVA with hemisphere (within subject) and treatment (between subject) factors were used to calculate statistical significance. All statistical analysis and figures were generated using GraphPad Prism 9.

Results

FIG. 2A depicts images of brain sections of mice treated with control (top panel) and mouse 3D6 [(m)PRX005); bottom panel]. Contralateral (contra) is on left side of each image, and ipsilateral (ipsi) is on right side of each image.

Results are shown in FIG. 2B. Overall tau pathology burden was lower in the contralateral (relative to injection) hippocampus compared to the ipsilateral hippocampus; this is expected as pathology in the contralateral hippocampus is due to tau propagation via efferent neurons from the injection site hippocampus. Systemic treatment with mouse 3D6 resulted in significant reductions in AT8 staining in both the ipsilateral and contralateral hippocampi as measured by immunohistochemistry (FIG. 2B) compared to treatment with the IgG2a isotype control. These results demonstrate the efficacy of systemically-administered mouse 3D6 in inhibiting the uptake and spread of tau pathology induced by AD-derived pathogenic species. All values are mean±SE (n=30).

### Example 3: Mouse 3D6 Treatment Reduces Pathological Tau and Ameliorates Behavior Deficit in a Transgenic Tau Model

Mouse 3D6 (mPRX005) efficacy was assessed in a transgenic tau aging model. Utilization of this model provides an orthogonal approach to testing efficacy, in that tau pathological development occurs due to aging and overexpression, and any tau species accessible to antibody treatment are secreted by neurons. This removed bias inherent in selection of the specific tau seed used in an induced disease model.

In this study age-dependent changes in pathology, posttranslational changes in tau, and behavioral changes were assessed in a human tau transgenic mouse line bearing the clinical frontotemporal dementia-related P301S mutation under control of the murine prion promoter (line PS19). The PS19 mice display an age-dependent tau hyperphosphorylation (as detected by AT8 and AT100) in spinal cord, brainstem, midbrain, cortex, amygdala, and hippocampus, as well as associated motor deficits. Tau pathological development is present by 6 months, and progresses along with concomitant neurodegeneration until death at 10-14 months of age.

Mice were treated with injection of PBS, IgG1 isotype control, and mouse 3D6 (mPRX005) weekly (50 mg/kg IP (intraperitoneal)) for 3 months (from 6-9.7 months of age), and various endpoints of tau pathology and associated behavioral deficits were measured.

Behavioral Assessment

The inverted grid hanging test was performed at 3, 6 and 9 months of age, and before sacrifice at 9.7 months of age. The inverted grid hanging tests coordination and muscle condition. The grid (40×20 cm/0.5×0.5 cm mesh) was positioned 50 cm above a flat, soft surface and the latency for the animal to drop down was measured.

Free-Floating Vibratome Sections

From each of the right hemispheres, a total of about 32 sagittal sections (40 μm) containing relevant regions of interest were cut. Brain sections of interest at intervals of 200 μm between bregma lateral 2.64 and 0.84 were selected based on a stereotaxic mouse brain atlas (Paxinos and Franklin). Sets of 5 sections per mouse and ROI were processed for staining with AT8 and AT100, respectively. Sections of all animals selected for a particular staining were randomized for staining and blinded quantified. Section number is mouse number with extension 1-5 for the different sections per mouse.

Immunohistochemical Procedures

Sagittal brain sections (40 m) were cut on a vibrating HM650V microtome (Thermo Scientific, Waltham, MA, USA) and were preserved in 1×PBS/sodium azide 0.1% until used.

Following washing with PBS twice for 5 minutes, the brain sections were incubated in a solution of 1×PBS: methanol (1:1) for 10 minutes, followed by 3 washes of 5 minutes each with PBS-0.1% Triton-100 (PBST). Following blocking (5% milk in PBST) for 30 minutes, the brain sections were incubated with the specific primary mouse anti-Tau antibody (AT8 or AT100, for specifications see Table 5 below) for 2 hours at room temperature (or overnight at 4° C.) followed, after 3 washes for 5 minutes with PBST, by incubation with the appropriate Alexa-conjugated secondary antibody in 5% milk-PBST (1:500; Invitrogen; ThermoFisher) for 1 hour at room temperature. Following 3 washes with PBST and 2 washes with PBS, 5 minutes each, the brain sections were mounted on microscope glass slides (Menzel, Superfrost+), dried, embedded with Fluoromount (Sigma-Aldrich) and cover slipped. Immunoreactive area in the ROI were determined with Image J. Statistical analysis was done in GraphPad Prism v9.0 using Kruskal-Wallis followed by Dunn's post hoc analysis (for multiple comparisons), where applicable. Outliers were identified using the ROUT method in GraphPad Prism based on the False Discovery Rate method (FDR) using a very stringent Q=0.1% (maximum desired FDR), where applicable.

TABLE 5
Summary of antibodies used for IHC analysis

| | | | | Stock | Working |
|---|---|---|---|---|---|
| mAb | Supplier | Specificity | Host | Conc. | Conc. |
| AT8 | Thermo | Human | Mouse | 200 μg/ml | 0.4 μg/ml |
| | Scientific | | | | |
| AT100 | Thermo | Human | Mouse | 200 μg/ml | 0.8 μg/ml |
| | Scientific | | | | |

Automatic Quantification

Images were acquired with a Leica DM400 B LED fluorescence microscope and analyzed with ImageJ. All acquired images were subjected to the same computer subroutines to minimize investigator bias. For quantification of AT8- and AT100-positive area, an automatic thresholding method was applied throughout analysis.

The regions of interest (middle of the rostral pons) was selected for brainstem quantification. For each staining with AT100 or AT8, five brain sections per mouse were included in the analysis, respectively, and the mean value was calculated. Images were manually corrected when possible or excluded when the region of interest included mechanical, structural, and/or staining artifacts.

FIG. 3, top panel, is a schematic of the transgenic tau model experimental protocol. [Tx: i.p. q1w 50 mpk refers to intraperitoneal injection, once a week, 50 mg per kg of mouse body weight]

Results

Systemic passive immunization with mouse 3D6 promoted the reduction of tau pathology in the brainstem (FIG. 3, bottom right panel; * p<0.05)) as measured by immunostaining with antibodies directed at sites of tau hyperphosphorylation. In addition, mouse 3D6 treatment reduced tau pathology-related motor deficits as measured by a grid-hanging assay (FIG. 3, bottom left panel; * p<0.05). Initiation of treatment at the onset of pathological development (treatment mode) with mPRX005 delays brainstem tau pathology and consequent behavioral deficits. Mouse 3D6 treatment also reduced tau pathological accumulation in the cortex and hippocampus, measured by immunohistochemical and biochemical techniques. Evaluation of trough antibody levels at two timepoints (before the 6^{th} dose and at study termination) indicated an average mouse 3D6 level of 280 μg/mL. Taken together, these results demonstrate efficacy with mouse 3D6 treatment in an aging transgenic model of tauopathy and provide confidence that mouse 3D6 treatment is effective against tau progression induced by non-fibrillar forms of tau.

### Example 4: Mouse 3D6 Protects Mouse Primary Cortical Neurons from Tau-Induced Toxicity

Cortical neurons from embryonic day 16-17 are prepared from C57B16/J mouse fetuses, as previously described (Pillot, T., Drouet, B., Queille, S., et al., The nonfibrillar amyloid beta-peptide induces apoptotic neuronal cell death: involvement of its C-terminal fusogenic domain. J Neurochem. 73(4):1626-34 (1999). In brief, dissociated cortical cells are plated (50,000 cells/well) in 48-well plates pre-coated with 1.5 μg/mL polyornithine (Sigma). Cells are cultured in a chemically defined Dulbecco's modified eagle's/F12 medium free of serum and supplemented with hormones, proteins and salts. Cultures are kept at 35° C. in a humidified 6% CO₂ atmosphere.

Neuron Treatments

All treatments were carried out in 48-well plates in triplicate at day 6 to 7 in vitro (DIV). Neurons were incubated either with vehicle or with human tau oligomers (hTO) (1 μM final concentration based on monomers) in the presence of 5 increasing concentrations of test item, for 24 h and in a final volume of 140 μL per well.

The final antibody to hTO ratios were: 1:5, 1:3, 1:1, 3:1 and 5:1, where hTO concentration is based on monomer molar equivalents, as the exact composition and epitope presentation of oligomers is unknown. The antibodies were incubated with hTO for 30 min at RT before addition to the neurons.

Measurement of Neuronal Viability

Mouse cortical neurons were incubated for 24 h following the addition of test conditions before monitoring neuronal viability using the 3-(4,5-dimethylthiazol-2-yl)-2,5diphenyltetrazoliumbromide (MTT) and lactate dehydrogenase (LDH)-release assays.

To measure MTT signal, cells were incubated at 35° C. for 1 h with MTT (Sigma, Cat #M2128-10G, Lot #MKBH7489V). For that purpose, MTT was solubilized in PBS at 5 mg/mL. 14 μL MTT solution were added to each well. After incubation, medium was removed and cells were lyzed with 150 μL DMSO for 10 minutes and protected from light. After complete solubilization of the formazan product, absorbance was determined at 570 nm in a FLUOSTAR-Omega plate reader (BMG-LABTECH).

For the measurement of LDH release, culture medium (110 μL) of each well was transferred into a 1.5 mL Eppendorf tube and replaced by fresh medium for the MTT assay. Collected medium was centrifuged at 800 g for five minutes and the supernatant (100 μL of cell-free culture medium) transferred into a 48-well plate stored at 4° C. and protected from light for further analysis. The quantification of LDH in culture medium was performed according to Manufacturer's recommendations (Cytotoxicity Detection Kit [LDH], Roche, Ref 11 644 793 001).

Results

Effect of Mouse 3D6 (mPRX005) on Neuronal Viability: LDH Assay

To test the ability of mouse 3D6 (mPRX005) to protect neurons from tau-induced neurotoxicity, primary mouse cortical neurons were treated with various concentrations of mouse 3D6 (mPRX005) with tau oligomers, and viability was measured by MTT assay. Molar equivalents of antibody:hTO were used, as (a) the specific composition and molecular weight of tau species are heterogeneous and unknown, and (b) due to limitations in experimental duration and differences between the in vitro and in vivo environment, the concentration of tau used in this particular model to induce measurable toxicity are greater than would be expected to be present in the extracellular environment in AD brain. Treatment with mouse 3D6 (mPRX005) reduced tau induced toxicity in a dose dependent manner, and returned neuronal viability to near-baseline levels at higher concentrations (FIG. 4, left panel; All values are mean±SD (n=3-5)).

Effect of Mouse 3D6 (mPRX005) on Neuronal Viability: LDH Assay

As an orthogonal method to assess neuronal viability, LDH release was also utilized to assess mouse 3D6 (mPRX005) prevention of tau-induced neurotoxicity. Lactate dehydrogenase (LDH) release is an indicator of cell death. Reduced LDH indicates reduced cell death, resulting from reduced internalization of tau. Similarly to treatment and results seen with the MTT assay, mouse 3D6 (mPRX005) demonstrated an ability to prevent neurotoxicity of tau in a dose dependent manner, indicating that the membrane integrity of neurons after treatment with tau was retained (FIG. 4, right panel; All values are mean±SD (n=3-5)).

In summary, in vitro screening of antibodies spanning the whole length of the tau protein indicated R1/R2 of MTBR displayed superior activity against tau uptake and neurotoxicity. The murine precursor of PRX005(mouse 3D6) has a high affinity for MTBR tau epitope and superior profile versus other antibodies. Direct inhibition of the tau-Heparan Sulfate ProteoGlycan interaction may contribute to blockade of tau internalization, toxicity, and development of intracellular tau pathology. In vivo treatment with mPRX005 (mouse 3D6) in transgenic tau mice and a seeding model reduces intraneuronal tau pathology and downstream behavioral deficits. The consistent, superior profile of PRX005 (hu3D6VHv1bA11/L2-DIM4) across a broad range of in vitro and in vivo systems supports advancing PRX005 (hu3D6VHv1bA11/L2-DIM4) as a clinical candidate for the potential treatment of tauopathies such as Alzheimer's disease.

### Example 5 Exemplary CDRs

Exemplary CDRs of antibodies of the invention are in Table 6.

TABLE 6
Exemplary CDRs

| | | SEQ | |
|---|---|---|---|
| | | ID | Exemplary VH or VL that CDR |
| CDR and Definition | CDR Amino Acid Sequence | NO: | is present in |
| Kabat/Chothia | GFNIKDYYLH | 8 | Mouse 3D6 VH |
| HCDR1 | | | |
| Kabat HCDR2 | WIDPENGDTVYDPKFQG | 9 | Mouse 3D6 VH |
| Kabat HCDR3: | LDF | 10 | Mouse 3D6 VH |
| Kabat LCDR1 | KSSQSLLDSDGKTYLN | 12 | Mouse 3D6 VL |
| Kabat LCDR2 | LVSKLDS | 13 | Mouse 3D6 VL |
| Kabat LCDR3: | WQGTHFPYT | 14 | Mouse 3D6 VL |
| CDR-H1 Kabat | DYYLH | 32 | Mouse 3D6 VH |
| CDR-H1 Chothia | GFNIKDY | 33 | Mouse 3D6 VH |
| CDR-H2 Chothia | DPENGD | 34 | Mouse 3D6 VH |
| CDR-H2 AbM | WIDPENGDTV | 35 | Mouse 3D6 VH |
| CDR-L1 Contact | KTYLNWL | 36 | Mouse 3D6 VL |
| CDR-L2 Contact | RLIYLVSKLD | 37 | Mouse 3D6 VL |
| CDR-L3 Contact | WQGTHEPY | 38 | Mouse 3D6 VL |
| CDR-H1 Contact | KDYYLH | 39 | Mouse 3D6 VH |
| CDR-H2 Contact | WIGWIDPENGDTV | 40 | Mouse 3D6 VH |
| CDR-H3 Contact | STLD | 41 | Mouse 3D6 VH |
| Kabat-Chothia | GFTIKDYYLH | 42 | hu3D6VHv5, |
| CDR-H1 | | | hu3D6VHv1bA11B6G2, |
| | | | hu3D6VHv1bA11B6H3, |
| | | | hu3D6VHv1e, and hu3D6VHv1f |
| Kabat CDR-H2 | WIDPEDGDTVYAPKFQG | 43 | hu3D6VHv5 and |
| | | | hu3D6VHv1bA11B6H3 |
| Kabat-Chothia | GFNFKDYYLH | 58 | hu3D6VH1c |
| CDR-H1 | | | |
| Kabat-Chothia | GYTFTDYYLH | 59 | hu3D6VHv1d, hu3D6VHv3c, |
| CDR-H1 | | | and hu3D6VHv4c |
| Kabat-Chothia | GYNFKDYYLH | 60 | hu3D6VHv3b and hu3D6VHv4b |
| CDR-H1 | | | |
| Kabat CDR-H2 | WVDPEDGDTVYAPKFQG | 61 | hu3D6VHv1bA11B6G2 |
| Kabat CDR-H2 | WIDPENGDTVYDEKFQG | 62 | hu3D6VHv1c, hu3D6VHv3b, |
| | | | and hu3D6VHv4b |
| Kabat CDR-H2 | WVDPEDGDTVYAEKFQG | 63 | hu3D6VHv1d, hu3D6VHv1f, |
| | | | hu3D6VHv3c, and |
| | | | hu3D6VHv4c |
| Kabat CDR-H2 | WIDPENGDTVYAEKFQG | 64 | hu3D6VHv1e |
| Kabat CDR-H3 | LDY | 65 | hu3D6VHv1f |
| Kabat/Chothia | GLNIKDYYIH | 67 | Mouse 6A10 VH |
| composite CDR- | | | |
| H1 | | | |
| Kabat CDR-H2 | WIDPENDDTEYAPKFQG | 68 | Mouse 6A10 VH |
| Kabat CDR-H3 | LDY | 69 | Mouse 6A10 VH |
| Kabat-Chothia | GFTIKDYYLH | 86 | hu3D6VHvb4 and hu3D6VHvb5 |
| Composite CDR- | | | |
| H1 | | | |
| Kabat CDR-H2 | WIDPENGDTIYDPKFQG | 87 | hu3D6VHvb3 and hu3D6VHvb4 |
| Kabat CDR-H2 | WIDPEDGETIYDPKFQG | 88 | hu3D6VHvb5 |
| Kabat CDR-L1 | RSSQSLLDSDGKTYLN | 89 | hu3D6VLvb3 |
| Kabat CDR-H2 | WIDPEDGETVYDPKFQG | 92 | hu3D6VHvb6 and hu3D6VHvb7 |
| Kabat CDR-H2 | WIDPENGDTVYEPKFQG | 149 | h3D6VHvb8 and h3D6VHvb9 |
| Kabat CDR-L2 | LVSKDDS | 150 | hu3D6VLv2 L54D and |
| | | | hu3D6VLv2 L37Q_L54D |
| Kabat CDR-L2 | LVSKGDS | 151 | hu3D6VLv2 L54G and |
| | | | hu3D6VLv2 L37Q_L54G |
| Kabat CDR-L2 | LVSKNDS | 152 | hu3D6VLv2 L54N |
| Kabat CDR-L2 | LVSKEDS | 153 | hu3D6VLv2 L54E and |
| | | | hu3D6VLv2 L37Q_L54E |
| Kabat CDR-L2 | EVSKLDS | 154 | hu3D6VLv2 L50E |
| Kabat CDR-L2 | LVSKQDS | 155 | hu3D6VLv2 L54Q |
| Kabat CDR-L2 | DVSKLDS | 156 | hu3D6VLv2 L50D and |
| | | | hu3D6VLv2 L37Q_L50D |
| Kabat CDR-L2 | LVSKKDS | 157 | hu3D6VLv2 L54K |
| Kabat CDR-L2 | LVSKRDS | 158 | hu3D6VLv2 L54R and |
| | | | hu3D6VLv2 L37Q_L54R |
| Kabat CDR-L2 | LVSKTDS | 159 | hu3D6VLv2 L54T and |
| | | | hu3D6VLv2 L37Q_L54T |
| Kabat CDR-L2 | GVSKLDS | 160 | hu3D6VLv2 L50G and |
| | | | hu3D6VLv2 L37Q_L50G |
| Kabat CDR-L2 | LVSKVDS | 161 | hu3D6VLv2 L54V |
| Kabat CDR-L2 | LVSKSDS | 162 | hu3D6VLv2 L54S |
| Kabat CDR-L2 | LVGKLDS | 163 | hu3D6VLv2 S52G and |
| | | | hu3D6VLv2 L37Q_S52G |
| Kabat CDR-L2 | VVSKLDS | 164 | hu3D6VLv2 L50V |
| Kabat CDR-L2 | GVSKRDS | 165 | hu3D6VLv2 L37Q_L50G_L54R |
| | | | and hu3D6VLv2 |
| | | | L37Q_L50G_L54R_G100Q |
| Kabat CDR-L2 | GVSKGDS | 166 | hu3D6VLv2 L37Q_L50G_L54G |
| | | | and hu3D6VLv2 |
| | | | L37Q_L50G_L54G G100Q |
| Kabat CDR-L2 | LVGKGDS | 167 | hu3D6VLv2 L37Q_S52G_L54G |
| Kabat CDR-L2 | LVGKRDS | 168 | hu3D6VLv2 L37Q_S52G_L54R |
| | | | and hu3D6VLv2 |
| | | | L37Q_S52G_L54R_G100Q |
| Kabat CDR-L2 | LVGKTDS | 169 | hu3D6VLv2 L37Q_S52G_L54T |
| Kabat CDR-L2 | LVGKDDS | 170 | hu3D6VLv2 L37Q_S52G_L54D |
| | | | and hu3D6VLv2 |
| | | | L37Q_S52G_L54D_G100Q |
| Kabat CDR-L2 | DVSKGDS | 171 | in hu3D6VLv2 |
| | | | L37Q_L50D_L54G and |
| | | | hu3D6VLv2 |
| | | | L37Q_L50D_L54G_G100Q |
| Kabat CDR-L2 | DVSKRDS | 172 | hu3D6VLv2 L37Q_L50D_L54R |
| | | | and hu3D6VLv2 |
| | | | L37Q_L50D_L54R_G100Q |
| Kabat CDR-L2 | EVSKGDS | 173 | hu3D6VLv2 L37Q_L50E_L54G |
| Kabat CDR-L2 | EVSKRDS | 174 | hu3D6VLv2 L37Q_L50E_L54R |
| Kabat CDR-L2 | VVSKDDS | 175 | hu3D6VLv2 |
| | | | L37Q_L50V_L54D_G100Q |

Listing of Sequences

P10636-8 (SEQ ID NO: 1)
MAEPRQEFEVMEDHAGTYGLGDRKDQGGYTMHQDQEGDTDAGLKESPLQTPTEDGSE
EPGSETSDAKSTPTAEDVTAPLVDEGAPGKQAAAQPHTEIPEGTTAEEAGIGDTPSLEDE
AAGHVTQARMVSKSKDGTGSDDKKAKGADGKTKIATPRGAAPPGQKGQANATRIPAK
TPPAPKTPPSSGEPPKSGDRSGYSSPGSPGTPGSRSRTPSLPTPPTREPKKVAVVRTPPKSP
SSAKSRLQTAPVPMPDLKNVKSKIGSTENLKHQPGGGKVQIINKKLDLSNVQSKCGSKD
NIKHVPGGGSVQIVYKPVDLSKVTSKCGSLGNIHHKPGGGQVEVKSEKLDFKDRVQSKI
GSLDNITHVPGGGNKKIETHKLTFRENAKAKTDHGAEIVYKSPVVSGDTSPRHLSNVSST
GSIDMVDSPQLATLADEVSASLAKQGL
P10636-7 (SEQ ID NO: 2)
MAEPRQEFEVMEDHAGTYGLGDRKDQGGYTMHQDQEGDTDAGLKESPLQTPTEDGSE
EPGSETSDAKSTPTAEAEEAGIGDTPSLEDEAAGHVTQARMVSKSKDGTGSDDKKAKG
ADGKTKIATPRGAAPPGQKGQANATRIPAKTPPAPKTPPSSGEPPKSGDRSGYSSPGSPG
TPGSRSRTPSLPTPPTREPKKVAVVRTPPKSPSSAKSRLQTAPVPMPDLKNVKSKIGSTEN
LKHQPGGGKVQIINKKLDLSNVQSKCGSKDNIKHVPGGGSVQIVYKPVDLSKVTSKCGS
LGNIHHKPGGGQVEVKSEKLDFKDRVQSKIGSLDNITHVPGGGNKKIETHKLTFRENAK
AKTDHGAEIVYKSPVVSGDTSPRHLSNVSSTGSIDMVDSPQLATLADEVSASLAKQGL
P10636-6 (4RON human tau) (SEQ ID NO: 3)
MAEPRQEFEVMEDHAGTYGLGDRKDQGGYTMHQDQEGDTDAGLKAEEAGIGDTPSLE
DEAAGHVTQARMVSKSKDGTGSDDKKAKGADGKTKIATPRGAAPPGQKGQANATRIP
AKTPPAPKTPPSSGEPPKSGDRSGYSSPGSPGTPGSRSRTPSLPTPPTREPKKVAVVRTPPK
SPSSAKSRLQTAPVPMPDLKNVKSKIGSTENLKHQPGGGKVQIINKKLDLSNVQSKCGS
KDNIKHVPGGGSVQIVYKPVDLSKVTSKCGSLGNIHHKPGGGQVEVKSEKLDFKDRVQ
SKIGSLDNITHVPGGGNKKIETHKLTFRENAKAKTDHGAEIVYKSPVVSGDTSPRHLSNV
SSTGSIDMVDSPQLATLADEVSASLAKQGL
P10636-5 (SEQ ID NO: 4)
MAEPRQEFEVMEDHAGTYGLGDRKDQGGYTMHQDQEGDTDAGLKESPLQTPTEDGSE
EPGSETSDAKSTPTAEDVTAPLVDEGAPGKQAAAQPHTEIPEGTTAEEAGIGDTPSLEDE
AAGHVTQARMVSKSKDGTGSDDKKAKGADGKTKIATPRGAAPPGQKGQANATRIPAK
TPPAPKTPPSSGEPPKSGDRSGYSSPGSPGTPGSRSRTPSLPTPPTREPKKVAVVRTPPKSP
SSAKSRLQTAPVPMPDLKNVKSKIGSTENLKHQPGGGKVQIVYKPVDLSKVTSKCGSLG
NIHHKPGGGQVEVKSEKLDFKDRVQSKIGSLDNITHVPGGGNKKIETHKLTFRENAKAK
TDHGAEIVYKSPVVSGDTSPRHLSNVSSTGSIDMVDSPQLATLADEVSASLAKQGL
P10636-4 (SEQ ID NO: 5)
MAEPRQEFEVMEDHAGTYGLGDRKDQGGYTMHQDQEGDTDAGLKESPLQTPTEDGSE
EPGSETSDAKSTPTAEAEEAGIGDTPSLEDEAAGHVTQARMVSKSKDGTGSDDKKAKG
ADGKTKIATPRGAAPPGQKGQANATRIPAKTPPAPKTPPSSGEPPKSGDRSGYSSPGSPG
TPGSRSRTPSLPTPPTREPKKVAVVRTPPKSPSSAKSRLQTAPVPMPDLKNVKSKIGSTEN
LKHQPGGGKVQIVYKPVDLSKVTSKCGSLGNIHHKPGGGQVEVKSEKLDFKDRVQSKI
GSLDNITHVPGGGNKKIETHKLTFRENAKAKTDHGAEIVYKSPVVSGDTSPRHLSNVSST
GSIDMVDSPQLATLADEVSASLAKQGL
P10636-2 (SEQ ID NO: 6)
MAEPRQEFEVMEDHAGTYGLGDRKDQGGYTMHQDQEGDTDAGLKAEEAGIGDTPSLE
DEAAGHVTQARMVSKSKDGTGSDDKKAKGADGKTKIATPRGAAPPGQKGQANATRIP
AKTPPAPKTPPSSGEPPKSGDRSGYSSPGSPGTPGSRSRTPSLPTPPTREPKKVAVVRTPPK
SPSSAKSRLQTAPVPMPDLKNVKSKIGSTENLKHQPGGGKVQIVYKPVDLSKVTSKCGS
LGNIHHKPGGGQVEVKSEKLDFKDRVQSKIGSLDNITHVPGGGNKKIETHKLTFRENAK
AKTDHGAEIVYKSPVVSGDTSPRHLSNVSSTGSIDMVDSPQLATLADEVSASLAKQGL
SEQ ID NO: 7; Murine 3D6 VH amino acid sequence:
EVQLQQSGADLVRPGALVKLSCKASGFNIKDYYLHWVRQRPEQGLEWIGWIDPENGDT
VYDPKFQGKATITADTSSNTAYLQLGSLTSEDTAVYFCSTLDFWGQGTTLTVSS
SEQ ID NO: 8; Kabat/Chothia HCDR1:
GFNIKDYYLH
SEQ ID NO: 9; Kabat HCDR2:
WIDPENGDTVYDPKFQG
SEQ ID NO: 10; Kabat HCDR3:
LDF
SEQ ID NO: 11; Murine 3D6 VL amino acid sequence:
DVVMTQTPLTLSVTIGQPASISCKSSQSLLDSDGKTYLNWLLQRPGQSPKRLIYLVSKLD
SGVPDRFTGSGSGTDFTLKISRVEAEDLGVYYCWQGTHFPYTFGGGTKLEIK
SEQ ID NO: 12; Murine Kabat LCDR1:
KSSQSLLDSDGKTYLN
SEQ ID NO: 13; Murine Kabat LCDR2:
LVSKLDS
SEQ ID NO: 14; Murine Kabat LCDR3:
WQGTHFPYT
SEQ ID NO: 15; hu3D6VHv1:
EVQLVQSGAEVVRPGALVKVSCKASGFNIKDYYLHWVRQAPEQGLEWIGWIDPENGDT
VYDPKFQGKATITADTSTNTAYLQLSSLTSEDTAVYFCSTLDFWGQGTLVTVSS
SEQ ID NO: 16; hu3D6VHv2:
EVQLVQSGAEVKKPGASVKVSCKVSGFNIKDYYLHWVRQAPEQGLEWMGWIDPENGD
TVYDPKFQGRVTITADTSTNTAYMELSSLTSEDTAVYYCSTLDFWGQGTLVTVSS
SEQ ID NO: 17; hu3D6VHv1b:
EVQLVQSGAEVVRPGALVKISCKASGFNIKDYYLHWVRQRPEQGLEWIGWIDPENGDT
VYDPKFQGKATITADTSTNTAYLQLGSLTSEDTAVYFCSTLDFWGQGTLVTVSS
SEQ ID NO: 18; hu3D6VHv1bA11:
EVQLVQSGAEVVKPGATVKISCKASGFNIKDYYLHWVRQRPGQGLEWIGWIDPENGDT
VYDPKFQGRATITADTSTDTAYLQLGSLTSEDTAVYFCSTLDFWGQGTLVTVSS
SEQ ID NO: 19; hu3D6VHv5:
EVQLVQSGAEVVKPGATVKISCKASGFTIKDYYLHWVRQRPGQGLEWIGWIDPEDGDT
VYAPKFQGRATITADTSTDTAYLQLGSLTSEDTAVYFCSTLDFWGQGTLVTVSS
SEQ ID NO: 20; hu3D6VLv1:
DVVMTQSPLSLSVTLGQPASISCKSSQSLLDSDGKTYLNWLLQRPGQSPKRLIYLVSKLD
SGVPDRFSGSGSGTDFTLKISRVEAEDVGVYYCWQGTHFPYTFGGGTKLEIK
SEQ ID NO: 21; hu3D6VLv2:
DVVMTQSPLSLPVTLGQPASISCKSSQSLLDSDGKTYLNWLLQRPGQSPRRLIYLVSKLD
SGVPDRFSGSGSGTDFTLKISRVEAEDVGVYYCWQGTHFPYTFGGGTKLEIK
SEQ ID NO: 22; hu3D6VLv3:
DVVMTQSPLSLPVTLGQPASISCKSSQSLLDSDGKTYLNWLLQRPGQSPRRLIYLVSKLD
SGVPSRFSGSGSGTDFTLKISRVEAEDVGVYYCWQGTHFPYTFGGGTKLEIK
SEQ ID NO: 23; hu3D6VLv4:
DIVMTQTPLSLSVTIGQPASISCKSSQSLLDSDGKTYLNWLLQKPGQSPKRLIYLVSKLDS
GVPDRFSGSGSGTDFTLKISRVEAEDVGVYYCWQGTHFPYTFGGGTKLEIK
SEQ ID NO: 24; heavy chain variable acceptor Acc. # BAC01986.1
QVQLQQSGAEVKKPGSSVKVSCKASGGTFGSYAISWVRQAPGQGLEWMGRIIPILGIAT
YAQKFQGRVTITADKSTSTAYMDLSSLRSEDTAVYYCARGKGEFEGMDVWGQGTTVT
VSS
SEQ ID NO: 25; heavy chain variable acceptor Acc. # IMGT# IGHV1-69-2*01
EVQLVQSGAEVKKPGATVKISCKVSGYTFTDYYMHWVQQAPGKGLEWMGLVDPEDG
ETIYAEKFQGRVTITADTSTDTAYMELSSLRSEDTAVYYCAT
SEQ ID NO: 26; heavy chain variable acceptor Acc. # IMGT#IGKJ1*01
QHWGQGTLVTVSS
SEQ ID NO: 27; light chain variable acceptor Acc. # IMGT#IGKV2-30*02 Acc. #
IMGT#IGKV2-30*02
DVVMTQSPLSLPVTLGQPASISCRSSQSLVHSDGNTYLNWFQQRPGQSPRRLIYKVSNRD
SGVPDRFSGSGSGTDFTLKISRVEAEDVGVYYCMQGTHWP
SEQ ID NO: 28; light chain variable acceptor Acc. # IMGT#IGKJ2*01
YTFGQGTKLEIK
SEQ ID NO: 29; Light chain variable acceptor Acc. # AAZ09048.1
DVVMTQSPLSLTVTLGQPASISCRSSQSLVYSDGNTYLNWFQQRPGQSPRRLIYRVSHW
DSGVPDRFSGSGSGTDFTLKISRVEAEDVGVYYCMQGTYWPLTFGQGTKLEIK
SEQ ID NO: 30; Murine 3D6 VH nucleic acid sequence:
GAGGTTCAGCTGCAGCAGTCTGGGGCTGACCTTGTGAGGCCAGGGGCCTTAGTCAA
GTTGTCCTGCAAAGCTTCTGGCTTCAACATTAAAGACTACTATTTGCACTGGGTGAG
GCAGAGGCCTGAACAGGGCCTGGAGTGGATTGGATGGATTGATCCTGAGAATGGTG
ATACTGTATATGACCCGAAGTTCCAGGGCAAGGCCACTATAACAGCAGACACATCC
TCCAATACAGCCTACCTGCAGCTCGGCAGCCTGACATCTGAGGACACTGCCGTCTAT
TTCTGTTCTACCCTTGACTTCTGGGGCCAAGGCACCACTCTCACAGTCTCCTCA
SEQ ID NO: 31; Murine 3D6 VL nucleic acid sequence:
GATGTTGTGATGACCCAGACTCCACTCACTTTGTCGGTTACCATTGGACAACCAGCC
TCCATCTCTTGCAAGTCAAGTCAGAGCCTCTTAGATAGTGATGGAAAGACATATTTG
AATTGGTTGTTACAGAGGCCAGGCCAGTCTCCAAAGCGCCTAATCTATCTGGTGTCT
AAACTGGACTCTGGAGTCCCTGACAGGTTCACTGGCAGTGGATCAGGGACAGATTTC
ACACTGAAAATCAGCAGAGTGGAGGCTGAGGATTTGGGAGTTTATTATTGCTGGCA
AGGTACACATTTTCCGTACACGTTCGGAGGGGGGACCAAGCTGGAAATAAAACGT
SEQ ID NO: 32; Murine CDR-H1 Kabat
DYYLH
SEQ ID NO: 33; Murine CDR-H1 Chothia
GFNIKDY
SEQ ID NO: 34; Murine CDR-H2 Chothia
DPENGD
SEQ ID NO: 35; Murine CDR-H2 AbM
WIDPENGDTV
SEQ ID NO: 36; Murine CDR-L1 Contact
KTYLNWL
SEQ ID NO: 37; Murine CDR-L2 Contact
RLIYLVSKLD
SEQ ID NO: 38; Murine CDR-L3 Contact
WQGTHEPY
SEQ ID NO: 39; Murine CDR-H1 Contact
KDYYLH
SEQ ID NO: 40; Murine CDR-H2 Contact
WIGWIDPENGDTV
SEQ ID NO: 41; Murine CDR-H3 Contact
STLD
SEQ ID NO: 42; Alternate Kabat-Chothia CDR-H1
GFTIKDYYLH
SEQ ID NO: 43; Alternate Kabat CDR-H2
WIDPEDGDTVYAPKFQG
SEQ ID NO: 44; consensus VH amino acid sequence from FIG. 2 of
PCT/IB2017/052544
EVQLVQSGAEVVXPGALVKISCKASGFNIKDYYLHWVRQRPEQGLEWIGWIDPENGDT
VYDPKFQGXATITADTSTNTAYLQLGSLTSEDTAVYFCSTLDFWGQGTLVTVSS
SEQ ID NO: 45; consensus VL amino acid sequence of FIG. 3 of
PCT/IB2017/052544
DVVMTQSPLSLSVTLGQPASISCKSSQSLLDSDGKTYLNWLLQRPGQSPKRLIYLVSKLD
SGVPDRFSGSGSGTDFTLKISRVEAEDVGVYYCWQGTHFPYTFGGGTKLEIKR
SEQ ID NO: 46; hu3D6VHv1bA11B6G2:
EVQLVQSGAEVVKPGATVKISCKASGFTIKDYYLHWVRQRPGKGLEWIGWVDPEDGDT
VYAPKFQGRATITADTSTDTAYLELGSLTSEDTAVYFCSTLDFWGQGTLVTVSS
SEQ ID NO: 47; hu3D6VHv1bA11B6H3:
EVQLVQSGAEVVKPGATVKISCKASGFTIKDYYLHWVRQRPGKGLEWIGWIDPEDGDT
VYAPKFQGRATITADTSTDTAYLELGSLTSEDTAVYFCSTLDFWGQGTLVTVSS
SEQ ID NO: 48; hu3D6VHv1c:
EVQLVQSGAEVKRPGALVKISCKASGFNFKDYYLHWVRQRPEQGLEWMGWIDPENGD
TVYDEKFQGRVTITADTSTNTAYLQLGSLTSEDTAVYFCSTLDFWGQGTLVTVSS
SEQ ID NO: 49; hu3D6VHv1d:
EVQLVQSGAEVKRPGALVKISCKASGYTFTDYYLHWVRQRPEQGLEWMGWVDPEDGD
TVYAEKFQGRVTITADTSTNTAYLQLGSLTSEDTAVYFCSTLDFWGQGTLVTVSS
SEQ ID NO: 50; hu3D6VHv1e:
EVQLVQSGADVvkPGALVKISCKASGFTIKDYYLHWVRQRPEQGLEWIGWIDPENGDTV
YAEKFQGRVTITADTSTNTAYLeLGSLTSEDTAVYFCSTLDFWGQGTTLTVSS
SEQ ID NO: 51; hu3D6VHv1f:
EVQLVQSGADVVKPGALVKISCKASGFTIKDYYLHWVRQRPGQGLEWIGWVDPEDGD
TVYAEKFQGRVTITADTSTDTAYMELGSLTSEDTAVYFCSTLDYWGQGTTLTVSS
SEQ ID NO: 52; hu3D6VHv3:
EVQLVQSGAEVKKPGATVKISCKVSGFNIKDYYLHWVRQAPGKGLEWMGWIDPENGD
TVYDPKFQGRVTITADTSTDTAYMELSSLRSEDTAVYYCSTLDFWGQGTLVTVSS
SEQ ID NO: 53; hu3D6VHv3b:
EVQLVQSGAEVKKPGALVKISCKVSGYNFKDYYLHWVRQAPGKGLEWMGWIDPENG
DTVYDEKFQGRVTITADTSTNTAYMELGSLRSEDTAVYYCSTLDFWGQGTLVTVSS
SEQ ID NO: 54; hu3D6VHv3c:
EVQLVQSGAEVKKPGALVKISCKVSGYTFTDYYLHWVRQAPGKGLEWMGWVDPEDG
DTVYAEKFQGRVTITADTSTNTAYMELGSLRSEDTAVYYCSTLDFWGQGTLVTVSS
SEQ ID NO: 55; hu3D6VHv4:
EVQLVQSGAEVVKPGATVKISCKVSGFNIKDYYLHWVRQRPGKGLEWIGWIDPENGDT
VYDPKFQGKATITADTSTNTAYLELGSLTSEDTAVYYCSTLDFWGQGTLVTVSS
SEQ ID NO: 56; hu3D6VHv4b:
EVQLVQSGAEVVKPGALVKISCKVSGYNFKDYYLHWVRQRPGKGLEWMGWIDPENGD
TVYDEKFQGRVTITADTSTDTAYLELGSLTSEDTAVYYCSTLDFWGQGTLVTVSS
SEQ ID NO: 57; hu3D6VHv4c:
EVQLVQSGAEVVKPGALVKISCKVSGYTFTDYYLHWVRQRPGKGLEWMGWVDPEDG
DTVYAEKFQGRVTITADTSTDTAYLELGSLTSEDTAVYYCSTLDFWGQGTLVTVSS
SEQ ID NO: 58; Alternate Kabat-Chothia CDR-H1 (as in hu3D6VH1c).
GFNFKDYYLH
SEQ ID NO: 59; Alternate Kabat-Chothia CDR-H1, (as in hu3D6VHv1d,
hu3D6VHv3c, and hu3D6VHv4c).
GYTFTDYYLH
SEQ ID NO: 60; Alternate Kabat-Chothia CDR-H1 (as in hu3D6VHv3b and
hu3D6VHv4b)
GYNFKDYYLH
SEQ ID NO: 61; Alternate Kabat CDR-H2 (as in hu3D6VHv1bA11B6G2).
WVDPEDGDTVYAPKFQG
SEQ ID NO: 62, Alternate Kabat CDR-H2 (as in hu3D6VHv1c, hu3D6VHv3b, AND
hu3D6VHv4b.
WIDPENGDTVYDEKFQG
SEQ ID NO: 63; Alternate Kabat CDR-H2 as in hu3D6VHv1d, hu3D6VHv1f,
hu3D6VHv3c, and hu3D6VHv4c).
WVDPEDGDTVYAEKFQG
SEQ ID NO: 64; Alternate Kabat CDR-H2 (as in hu3D6VHv1e).
WIDPENGDTVYAEKFQG
SEQ ID NO: 65; Alternate Kabat CDR-H3 (as in hu3D6VHv1f)
LDY
SEQ ID NO: 66; heavy chain variable region of the mouse 6A10 antibody.
EVQLQQSGAELVRSGASVKLSCTASGLNIKDYYIHWVKQRPEQGLEWIGWIDPENDDTE
YAPKFQGRATLTTDTSSNTAYLQLSSLTSEDTAVYYCTPLDYWGQGTSVTVSS
SEQ ID NO: 67; Kabat/Chothia composite CDR-H1 of the mouse 6A10 antibody.
GLNIKDYYIH
SEQ ID NO: 68; Kabat CDR-H2 of the mouse 6A10 antibody.
WIDPENDDTEYAPKFQG
SEQ ID NO: 69; Kabat CDR-H3 of the mouse 6A10 antibody
LDY
SEQ ID NO: 70; Mus VH structure template (PDB#1CR9_H)
KVKLQQSGAELVRSGASVKLSCTASGFNIKDYYIQWVKQRPEQGLEWIGWIDPENGNSEYAPRF
QGKATMTADTLSNTAYLQLSSLTSEDTAVYYCNADLHDYWGQGTTLTVSS
SEQ ID NO: 71; consensus VH amino acid sequence from FIGS. 4A and 4B of
PCT/IB2017/052544
EVQLVQSGAEVVKPGALVKISCKASGFNIKDYYLHWVRQRPGQGLEWIGWIDPENGDT
VYDPKFQGRVTITADTSTNTAYLELGSLTSEDTAVYFCSTLDFWGQGTLVTVSS
SEQ ID NO: 72; heavy chain of chimeric 3D6 antibody
EVQLQQSGADLVRPGALVKLSCKASGFNIKDYYLHWVRQRPEQGLEWIGWIDPENGDT
VYDPKFQGKATITADTSSNTAYLQLGSLTSEDTAVYFCSTLDFWGQGTTLTVSSASTKG
PSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMT
KNQVSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID NO: 73; light chain of chimeric 3D6 antibody
DVVMTQTPLTLSVTIGQPASISCKSSQSLLDSDGKTYLNWLLQRPGQSPKRLIYLVSKLD
SGVPDRFTGSGSGTDFTLKISRVEAEDLGVYYCWQGTHFPYTFGGGTKLEIKRTVAAPS
VFIFPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
SEQ ID NO: 74; amino acid sequence of heavy chain variable structural model Acc. #
5MYX-VH_mSt
EVQLQQSGAELVRPGSSVKISCKASGYIFNNYWINWVKQRPGQGLEWIGQIYPGDGDTN
YNGKFKGKATLTADKSSSTAYMQLSSLTSEDSAVYFCAREGYIVYWGQGTLVTVSA
SEQ ID NO: 75; amino acid sequence of heavy chain variable acceptor Acc. # 2RCS-
VH_huFrwk
QVQLQQSGAELVKPGASVKLSCTASGFNIKDTYMHWVKQRPEQGLEWIGRIDPANGNT
KYDPKFQGKATITADTSSNTAYLQLSSLTSEDTAVYYCASYYGIYWGQGTTLTVSS
SEQ ID NO: 76; amino acid sequence of heavy chain variable region of the humanized
3D6 antibody hu3D6VHvb1
QVQLQQSGAELVKPGASVKLSCTASGFNIKDYYLHWVKQRPEQGLEWIGWIDPENGDT
VYDPKFQGKATITADTSSNTAYLQLSSLTSEDTAVYFCSTLDFWGQGTTLTVSS
SEQ ID NO: 77; amino acid sequence of heavy chain variable region of the humanized
3D6 antibody hu3D6VHvb2
EVQLVQSGAEVVKPGASVKISCKASGFNIKDYYLHWVRQRPGKGLEWIGWIDPENGDT
VYDPKFQGRATITADTSTDTAYLELSSLTSEDTAVYFCSTLDFWGQGTLVTVSS
SEQ ID NO: 78; amino acid sequence of heavy chain variable region of the humanized
3D6 antibody hu3D6VHvb3
EVQLVQSGAEVVKPGATVKISCKASGFNIKDYYLHWVRQRPGKGLEWIGWIDPENGDTI
YDPKFQGRATITADTSTDTAYMELSSLRSEDTAVYYCSTLDFWGQGTLVTVSS
SEQ ID NO: 79; amino acid sequence of heavy chain variable region of the humanized
3D6 antibody hu3D6VHvb4
EVQLVQSGAEVVKPGATVKISCKASGFTIKDYYLHWVRQRPGKGLEWIGWIDPENGDTI
YDPKFQGRVTITADTSTDTAYMELSSLRSEDTAVYYCSTLDFWGQGTLVTVSS
SEQ ID NO: 80; amino acid sequence of heavy chain variable region of the humanized
3D6 antibody hu3D6VHvb5
EVQLVQSGAEVVKPGATVKISCKASGFTIKDYYLHWVRQRPGKGLEWIGWIDPEDGETI
YDPKFQGRVTITADTSTDTAYMELSSLRSEDTAVYYCSTLDFWGQGTLVTVSS
SEQ ID NO: 81; amino acid sequence of light chain variable structural model Acc. #
5MYX-VL_mSt
DVVLTQTPLTLSVTIGQPASISCKSSQSLLYSNGKTYLNWLLQRPGQSPKRLIYVVSKLDS
GVPDRFTGSGSGTDFTLKISRVEAEDLGVYYCVQGTHFPFTFGSGTKLEIK
SEQ ID NO: 82; amino acid sequence of light chain variable acceptor Acc. #
ARX71335-VL_huFrwk
DVVMTQTPLTLSVTIGQPASISCKSSQSLLYSNGKTYLNWLLQRPGQSPKRLIYLVSKLD
SGVPDRFSGSGSGTDFTLKISRVEAEDLGVHYCEQGTHFPLTFGAGTKLELK
SEQ ID NO: 83; amino acid sequence of light chain variable region of the humanized
3D6 antibody hu3D6VLvb1
DVVMTQTPLTLSVTIGQPASISCKSSQSLLDSDGKTYLNWLLQRPGQSPKRLIYLVSKLD
SGVPDRFSGSGSGTDFTLKISRVEAEDLGVHYCWQGTHFPYTFGAGTKLELK
SEQ ID NO: 84; amino acid sequence of light chain variable region of the humanized
3D6 antibody hu3D6VLvb2
DVVMTQSPLSLSVTLGQPASISCKSSQSLLDSDGKTYLNWLLQRPGQSPKRLIYLVSKLD
SGVPDRFSGSGSGTDFTLKISRVEAEDVGVYYCWQGTHFPYTFGAGTKLEIK
SEQ ID NO: 85; amino acid sequence of light chain variable region of the humanized
3D6 antibody hu3D6VLvb3
DVVMTQSPLSLSVTLGEPASISCRSSQSLLDSDGKTYLNWLQQRPGQSPRRLIYLVSKLD
SGVPDRFSGSGSGTDFTLKISRVEAEDVGVYYCWQGTHFPYTFGQGTKLEIK
SEQ ID NO: 86; amino acid sequence of an alternate Kabat-Chothia Composite CDR-
H1 of a humanized 3D6 antibody (as in hu3D6VHvb4 and hu3D6VHvb5)
GFTIKDYYLH
SEQ ID NO: 87; amino acid sequence of an alternate Kabat CDR-H2 of a humanized
3D6 antibody (as in hu3D6VHvb3 and hu3D6VHvb4)
WIDPENGDTIYDPKFQG
SEQ ID NO: 88; amino acid sequence of an alternate Kabat CDR-H2 of a humanized
3D6 antibody (as in hu3D6VHvb5)
WIDPEDGETIYDPKFQG
SEQ ID NO: 89; amino acid sequence of an alternate Kabat CDR-L1 of a humanized
3D6 antibody (as in hu3D6VLvb3)
RSSQSLLDSDGKTYLN
SEQ ID NO: 90; amino acid sequence of heavy chain variable region of the humanized
3D6 antibody hu3D6VHvb6
EVQLVQSGAEVVKPGATVKISCKASGFTIKDYYLHWVRQRPGKGLEWIGWIDPEDGET
VYDPKFQGRVTITADTSTDTAYMELSSLRSEDTAVYFCSTLDFWGQGTLVTVSS
SEQ ID NO: 91; amino acid sequence of heavy chain variable region of the humanized
3D6 antibody hu3D6VHvb7
EVQLVQSGAEVVKPGATVKISCKASGFTIKDYYLHWVRQRPGKGLEWIGWIDPEDGET
VYDPKFQGRVTITADTSTDTAYMELSSLRSEDTAVYYCSTLDFWGQGTLVTVSS
SEQ ID NO: 92; amino acid sequence of an alternate Kabat CDR-H2 of a humanized
3D6 antibody (as in hu3D6VHvb6 and hu3D6VHvb7)
WIDPEDGETVYDPKFQG
SEQ ID NO: 93; light chain variable region of a hu3D6VLv2 variant L54D, also known
as L2-DIM21
DVVMTQSPLSLPVTLGQPASISCKSSQSLLDSDGKTYLNWLLQRPGQSPRRLIYLVSKDDSGVP
DRFSGSGSGTDFTLKISRVEAEDVGVYYCWQGTHFPYTFGGGTKLEIK
SEQ ID NO: 94; light chain variable region of a hu3D6VLv2 variant L54G, also known
as L2-DIM7
DVVMTQSPLSLPVTLGQPASISCKSSQSLLDSDGKTYLNWLLORPGQSPRRLIYLVSKGDSGVP
DRFSGSGSGTDFTLKISRVEAEDVGVYYCWQGTHFPYTFGGGTKLEIK
SEQ ID NO: 95; light chain variable region of a hu3D6VLv2 variant L45N
DVVMTQSPLSLPVTLGQPASISCKSSQSLLDSDGKTYLNWLLQRPGQSPRRLIYLVSKNDSGVP
DRFSGSGSGTDFTLKISRVEAEDVGVYYCWQGTHFPYTFGGGTKLEIK
SEQ ID NO: 96; light chain variable region of a hu3D6VLv2 variant L54E
DVVMTQSPLSLPVTLGQPASISCKSSQSLLDSDGKTYLNWLLORPGQSPRRLIYLVSKEDSGVP
DRFSGSGSGTDFTLKISRVEAEDVGVYYCWQGTHFPYTFGGGTKLEIK
SEQ ID NO: 97; light chain variable region of a hu3D6VLv2 variant L50E
DVVMTQSPLSLPVTLGQPASISCKSSQSLLDSDGKTYLNWLLQRPGQSPRRLIYEVSKLDSGVP
DRFSGSGSGTDFTLKISRVEAEDVGVYYCWQGTHFPYTFGGGTKLEIK
SEQ ID NO: 98; light chain variable region of a hu3D6VLv2 variant L54Q
DVVMTQSPLSLPVTLGQPASISCKSSQSLLDSDGKTYLNWLLQRPGQSPRRLIYLVSKQDSGVP
DRFSGSGSGTDFTLKISRVEAEDVGVYYCWQGTHFPYTFGGGTKLEIK
SEQ ID NO: 99; light chain variable region of a hu3D6VLv2 variant L50D
DVVMTQSPLSLPVTLGQPASISCKSSQSLLDSDGKTYLNWLLQRPGQSPRRLIYDVSKLDSGVP
DRFSGSGSGTDFTLKISRVEAEDVGVYYCWQGTHFPYTFGGGTKLEIK
SEQ ID NO: 100; light chain variable region of a hu3D6VLv2 variant L54K
DVVMTQSPLSLPVTLGQPASISCKSSQSLLDSDGKTYLNWLLQRPGQSPRRLIYLVSKKDSGVP
DRFSGSGSGTDFTLKISRVEAEDVGVYYCWQGTHFPYTFGGGTKLEIK
SEQ ID NO: 101; light chain variable region of a hu3D6VLv2 variant L54R
DVVMTQSPLSLPVTLGQPASISCKSSQSLLDSDGKTYLNWLLQRPGQSPRRLIYLVSKRDSGVP
DRFSGSGSGTDFTLKISRVEAEDVGVYYCWQGTHFPYTFGGGTKLEIK
SEQ ID NO: 102; light chain variable region of a hu3D6VLv2 variant L54T
DVVMTQSPLSLPVTLGQPASISCKSSQSLLDSDGKTYLNWLLQRPGQSPRRLIYLVSKTDSGVP
DRFSGSGSGTDFTLKISRVEAEDVGVYYCWQGTHFPYTFGGGTKLEIK
SEQ ID NO: 103; light chain variable region of a hu3D6VLv2 variant L50G, also
known as L2-DIM22
DVVMTQSPLSLPVTLGQPASISCKSSQSLLDSDGKTYLNWLLQRPGQSPRRLIYGVSKLDSGVP
DRFSGSGSGTDFTLKISRVEAEDVGVYYCWQGTHFPYTFGGGTKLEIK
SEQ ID NO: 104; light chain variable region of a hu3D6VLv2 variant I48G
DVVMTQSPLSLPVTLGQPASISCKSSQSLLDSDGKTYLNWLLQRPGQSPRRLGYLVSKLDSGVP
DRFSGSGSGTDFTLKISRVEAEDVGVYYCWQGTHFPYTFGGGTKLEIK
SEQ ID NO: 105; light chain variable region of a hu3D6VLv2 variant I48D
DVVMTQSPLSLPVTLGQPASISCKSSQSLLDSDGKTYLNWLLORPGQSPRRLDYLVSKLDSGVP
DRFSGSGSGTDFTLKISRVEAEDVGVYYCWQGTHFPYTFGGGTKLEIK
SEQ ID NO: 106; light chain variable region of a hu3D6VLv2 variant L47G
DVVMTQSPLSLPVTLGQPASISCKSSQSLLDSDGKTYLNWLLQRPGQSPRRGIYLVSKLDSGVP
DRFSGSGSGTDFTLKISRVEAEDVGVYYCWQGTHFPYTFGGGTKLEIK
SEQ ID NO: 107; light chain variable region of a hu3D6VLv2 variant Y49E
DVVMTQSPLSLPVTLGQPASISCKSSQSLLDSDGKTYLNWLLQRPGQSPRRLIELVSKLDSGVP
DRFSGSGSGTDFTLKISRVEAEDVGVYYCWQGTHFPYTFGGGTKLEIK
SEQ ID NO: 108; light chain variable region of a hu3D6VLv2 variant L54V
DVVMTQSPLSLPVTLGQPASISCKSSQSLLDSDGKTYLNWLLQRPGQSPRRLIYLVSKVDSGVP
DRFSGSGSGTDFTLKISRVEAEDVGVYYCWQGTHFPYTFGGGTKLEIK
SEQ ID NO: 109; light chain variable region of a hu3D6VLv2 variant L54S
DVVMTQSPLSLPVTLGQPASISCKSSQSLLDSDGKTYLNWLLQRPGQSPRRLIYLVSKSDSGVP
DRFSGSGSGTDFTLKISRVEAEDVGVYYCWQGTHFPYTFGGGTKLEIK
SEQ ID NO: 110; light chain variable region of a hu3D6VLv2 variant S52G, also
known as L2-DIM9
DVVMTQSPLSLPVTLGQPASISCKSSQSLLDSDGKTYLNWLLQRPGQSPRRLIYLVGKLDSGVP
DRFSGSGSGTDFTLKISRVEAEDVGVYYCWQGTHFPYTFGGGTKLEIK
SEQ ID NO: 111; light chain variable region of a hu3D6VLv2 variant L47N
DVVMTQSPLSLPVTLGQPASISCKSSQSLLDSDGKTYLNWLLQRPGQSPRRNIYLVSKLDSGVP
DRFSGSGSGTDFTLKISRVEAEDVGVYYCWQGTHFPYTFGGGTKLEIK
SEQ ID NO: 112; light chain variable region of a hu3D6VLv2 variant L47D
DVVMTQSPLSLPVTLGQPASISCKSSQSLLDSDGKTYLNWLLORPGQSPRRDIYLVSKLDSGVP
DRFSGSGSGTDFTLKISRVEAEDVGVYYCWQGTHFPYTFGGGTKLEIK
SEQ ID NO: 113; light chain variable region of a hu3D6VLv2 variant L47E
DVVMTQSPLSLPVTLGQPASISCKSSQSLLDSDGKTYLNWLLQRPGQSPRREIYLVSKLDSGVP
DRFSGSGSGTDFTLKISRVEAEDVGVYYCWQGTHFPYTFGGGTKLEIK
SEQ ID NO: 114; light chain variable region of a hu3D6VLv2 variant L47P
DVVMTQSPLSLPVTLGQPASISCKSSQSLLDSDGKTYLNWLLORPGQSPRRPIYLVSKLDSGVP
DRFSGSGSGTDFTLKISRVEAEDVGVYYCWQGTHFPYTFGGGTKLEIK
SEQ ID NO: 115; light chain variable region of a hu3D6VLv2 variant L47T
DVVMTQSPLSLPVTLGQPASISCKSSQSLLDSDGKTYLNWLLQRPGQSPRRTIYLVSKLDSGVP
DRFSGSGSGTDFTLKISRVEAEDVGVYYCWQGTHFPYTFGGGTKLEIK
SEQ ID NO: 116; light chain variable region of a hu3D6VLv2 variant L47S
DVVMTQSPLSLPVTLGQPASISCKSSQSLLDSDGKTYLNWLLQRPGQSPRRSIYLVSKLDSGVP
DRFSGSGSGTDFTLKISRVEAEDVGVYYCWQGTHFPYTFGGGTKLEIK
SEQ ID NO: 117; light chain variable region of a hu3D6VLv2 variant L47A
DVVMTQSPLSLPVTLGQPASISCKSSQSLLDSDGKTYLNWLLQRPGQSPRRAIYLVSKLDSGVP
DRFSGSGSGTDFTLKISRVEAEDVGVYYCWQGTHFPYTFGGGTKLEIK
SEQ ID NO: 118, light chain variable region of a hu3D6VLv2 variant L50V
DVVMTQSPLSLPVTLGQPASISCKSSQSLLDSDGKTYLNWLLQRPGQSPRRLIYVVSKLDSGVP
DRFSGSGSGTDFTLKISRVEAEDVGVYYCWQGTHFPYTFGGGTKLEIK
SEQ ID NO: 119; light chain variable region of a hu3D6VLv2 variant
L37Q_L50G_L54R, also known as L2-DIM1
DVVMTQSPLSLPVTLGQPASISCKSSQSLLDSDGKTYLNWLQQRPGQSPRRLIYGVSKGDSGVP
DRFSGSGSGTDFTLKISRVEAEDVGVYYCWQGTHFPYTFGGGTKLEIK
SEQ ID NO: 120; light chain variable region of a hu3D6VLv2 variant
L37Q_L50G_LS4G, also known as L2-DIM2
DVVMTQSPLSLPVTLGQPASISCKSSQSLLDSDGKTYLNWLQQRPGQSPRRLIYEVSKGDSGVP
DRFSGSGSGTDFTLKISRVEAEDVGVYYCWQGTHFPYTFGGGTKLEIK
SEQ ID NO: 121; light chain variable region of a hu3D6VLv2 variant
L37Q_S52G_L54G, also known as L2-DIM3
DVVMTQSPLSLPVTLGQPASISCKSSQSLLDSDGKTYLNWLQQRPGQSPRRLIYLVGKGDSGVP
DRFSGSGSGTDFTLKISRVEAEDVGVYYCWQGTHFPYTFGGGTKLEIK
SEQ ID NO: 122; light chain variable region of a hu3D6VLv2 variant
L37Q_S52G_L54R, also known as L2-DIM4
DVVMTQSPLSLPVILGQPASISCKSSQSLLDSDGKTYLNWLQQRPGQSPRRLIYLVGKRDSGVP
DRFSGSGSGTDFTLKISRVEAEDVGVYYCWQGTHFPYTFGGGTKLEIK
SEQ ID NO: 123; light chain variable region of a hu3D6VLv2 variant
L37Q_S52G_L54T, also known as L2-DIM5
DVVMTQSPLSLPVTLGQPASISCKSSQSLLDSDGKTYLNWLQQRPGQSPRRLIYLVGKTDSGVP
DRFSGSGSGTDFTLKISRVEAEDVGVYYCWQGTHFPYTFGGGTKLEIK
SEQ ID NO: 124; light chain variable region of a hu3D6VLv2 variant
L37Q_S52G_L54D, also known as L2-DIM6
DVVMTQSPLSLPVTLGQPASISCKSSQSLLDSDGKTYLNWLQQRPGQSPRRLIYLVGKDDSGVP
DRFSGSGSGTDETLKISRVEAEDVGVYYCWQGTHFPYTFGGGTKLEIK
SEQ ID NO: 125; light chain variable region of a hu3D6VLv2 variant L37Q_L54R
DVVMTQSPLSLPVTLGQPASISCKSSQSLLDSDGKTYLNWLQQRPGQSPRRLIYLVSKGDSGVP
DRFSGSGSGTDFTLKISRVEAEDVGVYYCWQGTHFPYTFGGGTKLEIK
SEQ ID NO: 126; light chain variable region of a hu3D6VLv2 variant L37Q_L54G
DVVMTQSPLSLPVTLGQPASISCKSSQSLLDSDGKTYLNWLQQRPGQSPRRLIYLVSKGDSGVP
DRFSGSGSGTDFTLKISRVEAEDVGVYYCWQGTHFPYTFGGGTKLEIK
SEQ ID NO: 127; light chain variable region of a hu3D6VLv2 variant L37Q_LS4D,
also known as L2-DIM12
DVVMTQSPLSLPVTLGQPASISCKSSQSLLDSDGKTYLNWLQQRPGQSPRRLIYLVSKDDSGVP
DRFSGSGSGTDETLKISRVEAEDVGVYYCWQGTHFPYTFGGGTKLEIK
SEQ ID NO: 128; light chain variable region of a hu3D6VLv2 variant L37Q_L50G,
also known as L2-DIM13
DVVMTQSPLSLPVTLGQPASISCKSSQSLLDSDGKTYLNWLQQRPGQSPRRLIYGVSKLDSGVP
DRFSGSGSGTDFTLKISRVEAEDVGVYYCWQGTHFPYTFGGGTKLEIK
SEQ ID NO: 129; light chain variable region of a hu3D6VLv2 variant L37Q_L50D,
also known as L2-DIM14
DVVMTQSPLSLPVTLGQPASISCKSSQSLLDSDGKTYLNWLQQRPGQSPRRLIYDVSKLDSGVP
DRFSGSGSGTDFTLKISRVEAEDVGVYYCWQGTHFPYTFGGGTKLEIK
SEQ ID NO: 130; light chain variable region of a hu3D6VLv2 variant L37Q_L54T
DVVMTQSPLSLPVTLGQPASISCKSSQSLLDSDGKTYLNWLQQRPGQSPRRLIYDVSKLDSGVP
DRFSGSGSGTDFTLKISRVEAEDVGVYYCWQGTHFPYTFGGGTKLEIK
SEQ ID NO: 131; light chain variable region of a hu3D6VLv2 variant L37Q_S52G
DVVMTQSPLSLPVTLGQPASISCKSSQSLLDSDGKTYLNWLQQRPGQSPRRLIYLVGKLDSGVP
DRFSGSGSGTDFTLKISRVEAEDVGVYYCWQGTHFPYTFGGGTKLEIK
SEQ ID NO: 132; light chain variable region of a hu3D6VLv2 variant
L37Q_L50D_L54G, also known as L2-DIM17
DVVMTQSPLSLPVTLGQPASISCKSSQSLLDSDGKTYLNWLQQRPGQSPRRLIYDVSKGDSGVP
DRFSGSGSGTDFTLKISRVEAEDVGVYYCWQGTHFPYTFGGGTKLEIK
SEQ ID NO: 133; light chain variable region of a hu3D6VLv2 variant
L37Q_L50D_L54R, also known as L2-DIM18
DVVMTQSPLSLPVTLGQPASISCKSSQSLLDSDGKTYLNWLQQRPGQSPRRLIYDVSKRDSGVP
DRFSGSGSGTDFTLKISRVEAEDVGVYYCWQGTHFPYTFGGGTKLEIK
SEQ ID NO: 134; light chain variable region of a hu3D6VLv2 variant
L37Q_L50E_L54G, also known as L2-DIM19
DVVMTQSPLSLPVTLGQPASISCKSSQSLLDSDGKTYLNWLQQRPGQSPRRLIYEVSKGDSGVP
DRFSGSGSGTDFTLKISRVEAEDVGVYYCWQGTHFPYTFGGGTKLEIK
SEQ ID NO: 135; light chain variable region of a hu3D6VLv2 variant
L37Q_L50E_L54R, also known as L2-DIM20
DVVMTQSPLSLPVILGQPASISCKSSQSLLDSDGKTYLNWLQQRPGQSPRRLIYEVSKRDSGVP
DRFSGSGSGTDFTLKISRVEAEDVGVYYCWQGTHFPYTFGGGTKLEIK
SEQ ID NO: 136; light chain variable region of a hu3D6VLv2 variant
L37Q_L50G_L54R_G100Q
DVVMTQSPLSLPVILGQPASISCKSSQSLLDSDGKTYLNWLQQRPGQSPRRLIYGVSKRDSGVP
DRFSGSGSGTDFTLKISRVEAEDVGVYYCWQGTHFPYTFGQGTKLEIK
SEQ ID NO: 137; light chain variable region of a hu3D6VLv2 variant
L37Q_L50G_L54G_G100Q
DVVMTQSPLSLPVTLGQPASISCKSSQSLLDSDGKTYLNWLQQRPGQSPRRLIYGVSKGDSGVP
DRFSGSGSGTDFTLKISRVEAEDVGVYYCWQGTHFPYTFGOGTKLEIK
SEQ ID NO: 138; light chain variable region of a hu3D6VLv2 variant
L37Q_SS2G_L54R_G100Q
DVVMTQSPLSLPVTLGQPASISCKSSQSLLDSDGKTYLNWLQQRPGQSPRRLIYLVGKRDSGVP
DRFSGSGSGTDFTLKISRVEAEDVGVYYCWQGTHFPYTFGQGTKLEIK
SEQ ID NO: 139; light chain variable region of a hu3D6VLv2 variant
L37Q_SS2G_L54D_G100Q
DVVMTQSPLSLPVTLGQPASISCKSSOSLLDSDGKTYLNWLQQRPGQSPRRLIYLVGKDDSGVP
DRFSGSGSGTDFTLKISRVEAEDVGVYYCWQGTHFPYTFGQGTKLEIK
SEQ ID NO: 140; light chain variable region of a hu3D6VLv2 variant
L37Q_L50D_L54G_G100Q
DVVMTQSPLSLPVTLGQPASISCKSSQSLLDSDGKTYLNWLQQRPGQSPRRLIYDVSKGDSGVP
DRFSGSGSGTDFTLKISRVEAEDVGVYYCWQGTHFPYTFGQGTKLEIK
SEQ ID NO: 141; light chain variable region of a hu3D6VLv2 variant
L37Q_L50D_L54R_G100Q
DVVMTQSPLSLPVTLGQPASISCKSSQSLLDSDGKTYLNWLQQRPGQSPRRLIYDVSKRDSGVP
DRFSGSGSGTDFTLKISRVEAEDVGVYYCWQGTHFPYTFGQGTKLEIK
SEQ ID NO: 142; light chain variable region of a hu3D6VLv2 variant
L37Q_L50V_L54D_G100Q
DVVMTQSPLSLPVTLGQPASISCKSSQSLLDSDGKTYLNWLQQRPGQSPRRLIYVVSKDDSGVP
DRFSGSGSGTDFTLKISRVEAEDVGVYYCWQGTHFPYTFGQGTKLEIK
SEQ ID NO: 143; light chain variable region of a hu3D6VLv2 variant L37Q, also
known as L2-DIM8
DVVMTQSPLSLPVILGQPASISCKSSQSLLDSDGKTYLNWLQQRPGQSPRRLIYLVSKLDSGVP
DRFSGSGSGTDFTLKISRVEAEDVGVYYCWQGTHFPYTFGGGTKLEIK
SEQ ID NO: 144 light chain variable region of a hu3D6VLv2 variant G100Q
DVVMTQSPLSLPVILGQPASISCKSSQSLLDSDGKTYLNWLLQRPGQSPRRLIYLVSKLDSGVP
DRFSGSGSGTDFTLKISRVEAEDVGVYYCWQGTHFPYTFGQGTKLEIK
SEQ ID NO: 145 light chain variable region of a hu3D6VLv2 variant L37Q_L54E
DVVMTQSPLSLPVTLGQPASISCKSSQSLLDSDGKTYLNWLQQRPGQSPRRLIYLVSKEDSGVP
DRFSGSGSGTDFTLKISRVEAEDVGVYYCWQGTHFPYTFGGGTKLEIK
SEQ ID NO: 146; heavy chain variable region of a hu3D6VHv1bA11 variant D60E,
also known as h3D6VHvb8
EVQLVQSGAEVVKPGATVKISCKASGFNIKDYYLHWVRQRPGQGLEWIGWIDPENGDTVYEPKF
QGRATITADTSTDTAYLOLGSLTSEDTAVYFCSTLDFWGQGTLVTVSS
SEQ ID NO: 147 heavy chain variable region of a hu3D6VHv1bA11 variant L82cV
EVQLVQSGAEVVKPGATVKISCKASGFNIKDYYLHWVRQRPGQGLEWIGWIDPENGDTVYDPKF
QGRATITADTSTDTAYLQLGSVTSEDTAVYFCSTLDFWGQGTLVTVSS
SEQ ID NO: 148; heavy chain variable region of a hu3D6VHv1bA11 variant
D60E_L80M_Q81E_L82cV_T83R, also known as h3D6VHvb9
EVQLVQSGAEVVKPGATVKISCKASGFNIKDYYLHWVRORPGQGLEWIGWIDPENGDTVYEPKF
QGRATITADTSTDTAYMELGSVRSEDTAVYFCSTLDFWGQGTLVTVSS
SEQ ID NO: 149; amino acid sequence of an alternate Kabat CDR-H2 of a humanized
3D6 antibody (as in h3D6VHvb8 and in h3D6VHvb9)
WIDPENGDTVYEPKFQG
SEQ ID NO: 150; amino acid sequence of an alternate Kabat CDR-L2 of a humanized
3D6 antibody (as in hu3D6VLv2 L54D and in hu3D6VLv2 L37Q_L54D):
LVSKDDS
SEQ ID NO: 151; amino acid sequence of an alternate Kabat CDR-L2 of a humanized
3D6 antibody (as in hu3D6VLv2 L54G and in hu3D6VLv2 L37Q_L54G):
LVSKGDS
SEQ ID NO: 152; amino acid sequence of an alternate Kabat CDR-L2 of a humanized
3D6 antibody (as in hu3D6VLv2 L54N):
LVSKNDS
SEQ ID NO: 153; amino acid sequence of an alternate Kabat CDR-L2 of a humanized
3D6 antibody (as in hu3D6VLv2 L54E and in hu3D6VLv2 L37Q_L54E):
LVSKEDS
SEQ ID NO: 154; amino acid sequence of an alternate Kabat CDR-L2 of a humanized
3D6 antibody (as in hu3D6VLv2 L50E):
EVSKLDS
SEQ ID NO: 155; amino acid sequence of an alternate Kabat CDR-L2 of a humanized
3D6 antibody (as in hu3D6VLv2 L54Q):
LVSKQDS
SEQ ID NO: 156; amino acid sequence of an alternate Kabat CDR-L2 of a humanized
3D6 antibody (as in hu3D6VLv2 L50D and in hu3D6VLv2 L37Q_L50D):
DVSKLDS
SEQ ID NO: 157; amino acid sequence of an alternate Kabat CDR-L2 of a humanized
3D6 antibody (as in hu3D6VLv2 L54K):
LVSKKDS
SEQ ID NO: 158; amino acid sequence of an alternate Kabat CDR-L2 of a humanized
3D6 antibody (as in hu3D6VLv2 L54R and in hu3D6VLv2 L37Q_L54R):
LVSKRDS
SEQ ID NO: 159; amino acid sequence of an alternate Kabat CDR-L2 of a humanized
3D6 antibody (as in hu3D6VLv2 L54T and in hu3D6VLv2 L37Q_L54T):
LVSKTDS
SEQ ID NO: 160; amino acid sequence of an alternate Kabat CDR-L2 of a humanized
3D6 antibody (as in hu3D6VLv2 L50G and in hu3D6VLv2 L37Q_L50G):
GVSKLDS
SEQ ID NO: 161; amino acid sequence of an alternate Kabat CDR-L2 of a humanized
3D6 antibody (as in hu3D6VLv2 L54V):
LVSKVDS
SEQ ID NO: 162; amino acid sequence of an alternate Kabat CDR-L2 of a humanized
3D6 antibody (as in hu3D6VLv2 L54S):
LVSKSDS
SEQ ID NO: 163; amino acid sequence of an alternate Kabat CDR-L2 of a humanized
3D6 antibody (as in hu3D6VLv2 S52G and in hu3D6VLv2 L37Q_S52G):
LVGKLDS
SEQ ID NO: 164; amino acid sequence of an alternate Kabat CDR-L2 of a humanized
3D6 antibody (as in hu3D6VLv2 L50V):
VVSKLDS
SEQ ID NO: 165; amino acid sequence of an alternate Kabat CDR-L2 of a humanized
3D6 antibody (as in hu3D6VLv2 L37Q_L50G_L54R and hu3D6VLv2
L37Q_L50G_L54R_G100Q):
GVSKRDS
SEQ ID NO: 166; amino acid sequence of an alternate Kabat CDR-L2 of a humanized
3D6 antibody (as in hu3D6VLv2 L37Q_L50G_L54G and in hu3D6VLv2
L37Q_L50G_L54G_G100Q):
GVSKGDS
SEQ ID NO: 167; amino acid sequence of an alternate Kabat CDR-L2 of a humanized
3D6 antibody (as in hu3D6VLv2 L37Q_S52G_L54G):
LVGKGDS
SEQ ID NO: 168; amino acid sequence of an alternate Kabat CDR-L2 of a humanized
3D6 antibody (as in hu3D6VLv2 L37Q_S52G_L54R and in hu3D6VLv2
L37Q_S52G_L54R_G100Q):
LVGKRDS
SEQ ID NO: 169; amino acid sequence of an alternate Kabat CDR-L2 of a humanized
3D6 antibody (as in hu3D6VLv2 L37Q_S52G_L54T):
LVGKTDS
SEQ ID NO: 170; amino acid sequence of an alternate Kabat CDR-L2 of a humanized
3D6 antibody (as in hu3D6VLv2 L37Q_S52G_L54D and in hu3D6VLv2
L37Q_S52G_L54D_G100Q):
LVGKDDS
SEQ ID NO: 171; amino acid sequence of an alternate Kabat CDR-L2 of a humanized
3D6 antibody (as in hu3D6VLv2 L37Q_L50D_L54G and in hu3D6VLv2
L37Q_L50D_L54G_G100Q):
DVSKGDS
SEQ ID NO: 172; amino acid sequence of an alternate Kabat CDR-L2 of a humanized
3D6 antibody (as in hu3D6VLv2 L37Q_L50D_L54R and in hu3D6VLv2
L37Q_L50D_L54R_G100Q):
DVSKRDS
SEQ ID NO: 173; amino acid sequence of an alternate Kabat CDR-L2 of a humanized
3D6 antibody (as in hu3D6VLv2 L37Q_L50E_L54G):
EVSKGDS
SEQ ID NO: 174; amino acid sequence of an alternate Kabat CDR-L2 of a humanized
3D6 antibody (as in hu3D6VLv2 L37Q_L50E_L54R):
EVSKRDS
SEQ ID NO: 175; amino acid sequence of an alternate Kabat CDR-L2 of a humanized
3D6 antibody (as in hu3D6VLv2 L37Q_L50V_L54D_G100Q):
VVSKDDS
SEQ ID NO: 176; amino acid sequence of a heavy chain constant region (IgG1: allotype
G1m17, 1):
ASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHEALHNHYT
QKSLSLSPGK
SEQ ID NO: 177; amino acid sequence of a light chain constant region (kappa):
RTVAAPSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
SEQ ID NO: 178; amino acid sequence of a mature heavy chain
of a 3D6 humanized variant (hu3D6VHv1bA11 IgG1 G1m17 allotype)
EVQLVQSGAEVVKPGATVKISCKASGFNIKDYYLHWVRQRPGQGLEWIGWIDPENGDTVYDPKF
QGRATITADTSTDTAYLQLGSLTSEDTAVYFCSTLDFWGQGTLVTVSSASTKGPSVFPLAPSSK
STSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKPKDTLMISRTPEVTC
VVVDVSHEDPEVKFNWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQKSLSLSPGK
SEQ ID NO: 179; amino acid sequence of a mature light chain
of a 3D6 humanized variant (hu3D6VLv2 variant L37Q_S52G_L54R,
L2-DIM4 kappa)
DVVMTQSPLSLPVTLGQPASISCKSSQSLLDSDGKTYLNWLQQRPGQSPRRLIYLVGKRDSGVP
DRFSGSGSGTDFTLKISRVEAEDVGVYYCWQGTHFPYTFGGGTKLEIKRTVAAPSVFIFPPSDE
QLKSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 180; amino acid sequence of a heavy chain of a
3D6 humanized variant (hu3D6VHv1bA11 IgG1 G1m17 allotype) with
bovine alpha-lactalbumin signal peptide at the N-terminus
MMSFVSLLLVGILFHATQAEVQLVQSGAEVVKPGATVKISCKASGFNIKDYYLHWVRQRPGQGL
EWIGWIDPENGDTVYDPKFQGRATITADTSTDTAYLQLGSLTSEDTAVYFCSTLDFWGQGTLVT
VSSASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEALHN
HYTQKSLSLSPGK
SEQ ID NO: 181; amino acid sequence of a light chain of a
3D6 humanized variant (hu3D6VLv2 variant L37Q_S52G_L54R, L2-DIM4
kappa) with bovine alpha-lactalbumin signal peptide at the N-
terminus.
MMSFVSLLLVGILFHATQADVVMTQSPLSLPVTLGQPASISCKSSQSLLDSDGKTYLNWLQORP
GQSPRRLIYLVGKRDSGVPDRFSGSGSGTDFTLKISRVEAEDVGVYYCWQGTHFPYTFGGGTKL
EIKRTVAAPSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 182; nucleotide sequence encoding a heavy chain
of a 3D6 humanized variant (hu3D6VHv1bA11 IgG1 G1m17 allotype)
with bovine alpha-lactalbumin signal peptide at the N-terminus
ATGATGTCCTTTGTCTCTCTGCTCCTGGTTGGCATCCTATTCCATGCCACCCAGGCCGAGGTGC
AGCTGGTGCAGTCTGGGGCAGAGGTTGTGAAGCCAGGGGCCACAGTCAAGATCTCCTGTAAGGC
TTCTGGCTTCAACATTAAAGACTACTATCTGCACTGGGTGCGGCAGAGGCCTGGACAGGGCCTG
GAGTGGATTGGATGGATTGATCCTGAGAATGGTGATACTGTGTATGACCCGAAGTTCCAGGGCA
GGGCCACTATAACAGCAGACACATCCACCGACACAGCCTACCTGCAGCTCGGCAGCCTGACATC
TGAGGACACTGCCGTCTATTTCTGTTCTACCCTGGACTTCTGGGGCCAAGGCACCCTTGTCACA
GTCTCCTCAGCCTCCACCAAGGGCCCATCGGTCTTCCCCCTGGCACCCTCTAGCAAGAGCACCT
CTGGGGGCACAGCGGCCCTGGGCTGCCTGGTCAAGGACTACTTCCCCGAACCGGTGACGGTGTC
GTGGAACTCAGGCGCCCTGACCAGCGGCGTGCACACCTTCCCGGCTGTCCTACAGTCCTCAGGA
CTCTACTCCCTCAGCAGCGTGGTGACCGTGCCCTCCAGCAGCTTGGGCACCCAGACCTACATCT
GCAACGTGAATCACAAGCCCAGCAACACCAAGGTGGACAAGAAGGTTGAGCCCAAATCTTGTGA
CAAAACTCACACATGCCCACCGTGCCCAGCACCTGAACTCCTGGGGGGACCGTCAGTCTTCCTC
TTCCCCCCAAAACCCAAGGACACCCTCATGATCTCCCGGACCCCTGAGGTCACATGCGTGGTGG
TGGACGTGAGCCACGAAGACCCTGAGGTCAAGTTCAACTGGTACGTGGACGGCGTGGAGGTGCA
TAATGCCAAGACAAAGCCGAGAGAGGAGCAGTACAACAGCACGTACCGTGTGGTCAGCGTCCTC
ACCGTCCTGCACCAGGACTGGCTGAATGGCAAGGAGTACAAGTGCAAGGTCTCCAACAAAGCCC
TCCCAGCCCCCATCGAGAAAACCATCTCCAAAGCCAAAGGGCAGCCCCGAGAACCACAGGTGTA
CACCCTGCCCCCATCCCGGGAGGAGATGACCAAGAACCAGGTCAGCCTGACCTGCCTGGTCAAA
GGCTTCTATCCCAGCGACATCGCCGTGGAGTGGGAGAGCAATGGGCAGCCGGAGAACAACTACA
AGACCACGCCTCCCGTGCTGGACTCCGACGGCTCCTTCTTCCTCTATTCCAAACTCACCGTGGA
CAAGAGCAGGTGGCAGCAGGGGAACGTCTTCTCATGCTCCGTGATGCATGAGGCTCTGCACAAC
CACTACACGCAGAAGAGCCTCTCCCTGTCTCCCGGGAAATGATGAGATCTCGAG
SEQ ID NO: 183; nucleotide sequence encoding a light chain
of a 3D6 humanized variant (hu3D6VLv2 variant L37Q_S52G_L54R,
L2-DIM4 kappa) with bovine alpha-lactalbumin signal peptide at
the N-terminus
ATGATGTCCTTTGTCTCTCTGCTCCTGGTTGGCATCCTATTCCATGCCACCCAGGCCGATGTTG
TGATGACCCAGTCTCCACTCTCTTTGCCCGTTACCCTTGGACAACCTGCCTCCATCTCTTGCAA
GTCAAGTCAGAGCCTCTTAGATAGTGATGGAAAGACATATTTGAATTGGTTGCAACAGAGGCCA
GGCCAGTCTCCACGGCGCCTAATCTATCTGGTGGGCAAACGGGACTCTGGAGTCCCTGACAGGT
TCAGTGGCAGTGGATCAGGGACAGATTTCACACTGAAAATCAGCAGAGTGGAGGCTGAGGATGT
GGGAGTTTATTATTGCTGGCAAGGCACACATTTTCCGTACACGTTCGGAGGGGGGACCAAGCTG
GAAATAAAACGAACTGTGGCTGCACCATCTGTCTTCATCTTCCCGCCATCTGATGAGCAGCTTA
AGTCCGGAACTGCTAGCGTTGTGTGCCTGCTGAATAACTTCTATCCCAGAGAGGCCAAAGTACA
GTGGAAGGTGGATAACGCCCTCCAATCGGGTAACTCCCAGGAGAGTGTCACAGAGCAGGACAGC
AAGGACAGCACCTACAGCCTCAGCAGCACCCTGACGCTGAGCAAAGCAGACTACGAGAAACACA
AAGTCTACGCCTGCGAAGTCACCCATCAGGGCCTGAGCTCGCCCGTCACAAAGAGCTTCAACAG
GGGAGAGTGTTAGTGAGATCTCGAG
SEQ ID NO: 184; amino acid sequence of a region of tau microtubule binding repeat 1
(amino acid residues 255-271 of SEQ ID NO: 1)
NVKSKIGSTENLKHQPG
SEQ ID NO: 185; amino acid sequence of of a region of tau microtubule binding repeat 2
(amino acid residues 286-302 of SEQ ID NO: 1)
NVQSKCGSKDNIKHVPG
SEQ ID NO: 186; amino acid sequence of of a region of tau microtubule binding repeat 3
(amino acid residues 317-333 of SEQ ID NO: 1)
KVTSKCGSLGNIHHKPG
SEQ ID NO: 187; amino acid sequence of of a region of tau microtubule binding repeat 4
(amino acid residues 349-365 of SEQ ID NO: 1)
RVQSKIGSLDNITHVPG
SEQ ID NO: 188; amino acid sequence of a core motif of tau bound by 3D6
KIGSTENLKH
SEQ ID NO: 189; amino acid sequence of tau sequence N-terminal to a core motif of tau
bound by 3D6
NVKS
SEQ ID NO: 190; amino acid sequence of tau sequence C-terminal to core motif of tau
bound by 3D6
QPG
SEQ ID NO: 191; amino acid sequence of epitope of 3D6
KXXSXXNX(K/H)H
SEQ ID NO: 192; amino acid sequence of a core motif of tau bound by 3D6
KCGSKDNIKH
SEQ ID NO: 193; amino acid sequence of a core motif of tau bound by 3D6
KCGSLGNIHH
SEQ ID NO: 194; amino acid sequence of a core motif of tau bound by 3D6
KIGSLDNITH

What is claimed is:

1. A method of reducing internalization of tau by cells in a subject comprising administering to a subject in need thereof an amount of an antibody or an antigen-binding fragment thereof that reduces internalization of tau by cells, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO:14.

2. A method of reducing tau induced toxicity in a subject comprising administering to a subject in need thereof an amount of an antibody or an antigen-binding fragment thereof that reduces tau induced toxicity, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO: 14.

3. A method of reducing or delaying onset of behavioral deficit in a subject comprising administering to a subject in need thereof an amount of an antibody or an antigen-binding fragment thereof that reduces or delays onset of behavioral deficit, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO: 14.

4. A method of reducing levels of markers of tau pathology in a subject comprising administering to a subject in need thereof an amount of an antibody or an antigen-binding fragment thereof that reduces markers of tau pathology, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO:14.

5. A method of reducing development of tau pathology in a subject comprising administering to a subject in need thereof an amount of an antibody or an antigen-binding fragment thereof that reduces tau pathology, wherein the antibody or the antigen-binding fragment thereof comprises a heavy chain variable domain comprising CDR-H1 comprising SEQ ID NO:8, CDR-H2 comprising SEQ ID NO:9, and CDR-H3 comprising LDF, and a light chain variable domain comprising CDR-L1 comprising SEQ ID NO:12, CDR-L2 comprising SEQ ID NO:13 or SEQ ID NO:168, and CDR-L3 comprising SEQ ID NO: 14.

6. A method of any one of the preceding claims wherein the subject has pathological features of Alzheimer's disease.

7. A method of any one of the preceding claims wherein the subject has Alzheimer's disease.

8. A method of any one of the preceding claims, wherein the CDR-L2 of the antibody or antigen-binding fragment comprises SEQ ID NO:13.

9. A method of any one of the preceding claims, wherein the CDR-L2 of the antibody or antigen-binding fragment comprises SEQ ID NO:168.

10. A method of any one of the preceding claims, wherein the heavy chain variable region of the antibody or antigen-binding fragment comprises a mature heavy chain variable region of SEQ ID NO:18 and the light chain variable region of the antibody or antigen-binding fragment comprises a mature light chain variable region of SEQ ID NO: 122.

11. A method of any one of the preceding claims, wherein the antibody or antigen-binding fragment is a humanized version of a mouse antibody characterized by a mature heavy chain variable region of SEQ ID NO: 7 and a mature light chain variable region of SEQ ID NO:11.

12. A method of any one of the preceding claims, wherein the antibody comprises a light chain comprising the mature light chain variable region fused to a light chain constant region and a heavy chain comprising the mature heavy chain variable region fused to a heavy chain constant region.

13. The method of claim 12, wherein the heavy chain constant region of the antibody comprises the amino acid sequence of SEQ ID NO:176 with or without the C-terminal lysine.

14. The method of claim 12, wherein the mature heavy chain variable region fused to the heavy chain constant region comprises the amino acid sequence of SEQ ID NO:178 with or without the C-terminal lysine.

15. The method of claim 12, wherein the antibody further comprises a signal peptide fused to the mature heavy and/or light chain variable region.

16. The method of claim 15, wherein the heavy chain comprises the amino acid sequence of SEQ ID NO:180 with or without C-terminal lysine.

17. The method of claim 12, wherein the light chain constant region of the antibody comprises the amino acid sequence of SEQ ID NO:177.

18. The method of claim 12, wherein the mature light chain variable region fused to a light chain constant region comprises the amino acid sequence of SEQ ID NO:179.

19. The method of claim 18, wherein the light chain comprises the amino acid sequence of SEQ ID NO:181.

20. The method of claim 14, wherein the heavy chain comprises the amino acid sequence of SEQ ID NO:178 with or without the C-terminal lysine and the light chain comprises the amino acid sequence of SEQ ID NO:179.

21. The method of claim 16, wherein the heavy chain comprises the amino acid sequence of SEQ ID NO:180 with or without the C-terminal lysine and the light chain comprises the amino acid sequence of SEQ ID NO:181.

22. The method of claim 12 wherein the antibody comprise at least one mutation in the constant region.

23. The method of claim 22, wherein the antibody comprise at least one mutation in the constant region, wherein the mutation reduces complement fixation or activation by the constant region or reduces binding to a Fcγ receptor relative to the natural human heavy chain constant region.

24. The method of claim 23 wherein the antibody comprises a mutation at one or more of positions 241, 264, 265, 270, 296, 297, 318, 320, 322, 329 and 331 by EU numbering or alanine at positions 318, 320 and 322.
